(19) United States
(12) Patent Application Publication
(10) Pub. No.: US 2024/0156963 A1
(43) Pub. Date: May 16, 2024

(54) COMBINATIONAL IMMUNOTHERAPIES USING CAR-M, CAR-NK, CAR-EOS, AND CAR-N CELLS

(71) Applicant: Wisconsin Alumni Research Foundation, Madison, WI (US)

(72) Inventors: James Thomson, Madison, WI (US); Jue Zhang, Madison, WI (US); Igor Slukvin, Verona, WI (US); Aditi Majumder, Madison, WI (US); Ho Sun Jung, Cambridge, MA (US)

(21) Appl. No.: 18/302,636

(22) Filed: Apr. 18, 2023

Related U.S. Application Data
(60) Provisional application No. 63/332,225, filed on Apr. 18, 2022.
(60) Provisional application No. 63/384,764, filed on Nov. 22, 2022.

(51) Int. Cl. A61K 39/00; A61P 35/00; C07K 14/725; C07K 16/30; C12N 5/0783; C12N 5/0786; C12N 5/0787

(52) U.S. Cl. CPC A61K 39/464471; A61K 39/461; A61K 39/4613; A61K 39/4614; A61K 39/4631; A61P 35/00; C07K 14/7051; C07K 16/3084; C12N 5/0642; C12N 5/0645; C12N 5/0646; C12N 2506/45; C12N 2510/00

(57) ABSTRACT

This disclosure provides genetically engineered immune cells that express an anti-GD2 chimeric antigen receptor, methods of generating these cells, and methods of treating tumors using the genetically engineered cells.

### RELATED APPLICATIONS

This application claims priority to U.S. provisional application No. 63/332,225, filed Apr. 18, 2022, and U.S. provisional application No. 63/384,764, filed Nov. 22, 2022, the disclosures of which are expressly incorporated by reference herein.

### STATEMENT REGARDING FEDERALLY SPONSORED RESEARCH

This invention was made with government support under U01HL134655, OD0101106, and HL142665 awarded by the National Institute of Health. The government has certain rights in the invention.

### INCORPORATION BY REFERENCE OF SEQUENCE LISTING PROVIDED ELECTRONICALLY

This application contains a Sequence Listing submitted as an electronic text file named “21-1222-US_Sequence-Listing.xml,” having a size in bytes of 16.7 kb, and created on Apr. 18, 2023. The information contained in this electronic file is hereby incorporated by reference in its entirety.

### BACKGROUND OF THE DISCLOSURE

Chimeric antigen receptor T (CAR-T) cell and chimeric antigen receptor natural killer (CAR-NK) cell therapies have already demonstrated tremendous success in the eradication of lymphoid malignancies. While these therapies demonstrate high efficacy in eliminating liquid cancers (lymphoma, leukemia), many challenges remain in the applying CAR therapies for solid tumors. Thus, there is a significant need for cellular therapies effective against solid tumors. Limited traffic of CAR-lymphocytes to the tumor and loss of activity of CAR-lymphocytes within immunosuppressive tumor environments are believed to be the major factors contributing to the incapability of CAR-T cells to kill solid tumors.

Neutrophils and macrophages provide a logical alternative for cellular immunotherapy since they can migrate to, and into, solid tumors better than any other cell type and can kill tumor cells by perforin/granzyme-independent mechanisms allowing targeting of perforin/granzyme resistant tumors. Human pluripotent stem cell-derived hematopoietic cells can further be differentiated into lymphocytes cells for anti-cancer immunotherapy (Li et al., 2018, Cell Stem Cell 23:181-192 e185; Themeli et al., 2013, Nat. Biotechnol. 31:928-933). Unfortunately, antitumor efficacy of such cells is usually limited by poor infiltration, deficient expansion, inferior survival, and inactivation of tumor-targeted lymphocytes in solid tumors (Guedan et al., 2019, Annu. Rev. Immunol. 37:145-171; Morrissey et al., 2018, eLife 7).

The interaction between white blood cells plays a key role in immune responses. For example, natural killer (NK) cells and macrophages form a major first-line defense against pathogens (bacteria, viruses, fungi, and parasites). Macrophages can activate NK cells through direct cell-to-cell contact and through soluble cytokines such as IL-12, IL15, and IL-18. NK cells secrete IFNγ, which in turn activates macrophages. The positive feedback between macrophages and NK cells increases the activation of both types of cells.

Primary macrophages and neutrophils usually have limited and heterogeneous cell sources, which are not suitable for generating an off-the-shelf universal cell therapy. Macrophages can be classified into M1 anti-cancer/pro-inflammatory and M2 pro-cancer/anti-inflammatory types (Martinez and Gordon, 2014, F1000Prime Rep 6: 13; Owen and Mohamadzadeh, 2013, Front. Physiol. 4:159). The failure of clinical trials of natural macrophages has been attributed generally to inactivation of the antitumor activity (Lee et al., 2016, J. Control Release. 240:527-540).

Genetic modification of somatic macrophages and neutrophils remains a significant challenge. On the other hand, human pluripotent stem cells (hPSCs) provide an unlimited homogeneous source for cell therapy. hPSCs are amendable to multiplex gene editing to enable the generation of CAR-expressing immune cells and hypoimmunogenic cells for universal cell therapy (Deuse et al., 2019, Nat. Biotechnol. 37:252-258; Gornalusse et al., 2017, Nat. Biotechnol. 35:765-772; Xu et al., 2019, Cell Stem Cell 24:566-578 e567).

Methods for differentiating neutrophils from precursor cells were disclosed in Chang et al., 2022, Cell Reports 40: 111128 and in PCT WO2022125850A1, that used a chemically defined, feeder cell-free platform that applied stage-specific signaling modulators for differentiation. These cells were then engineered to display CAR having a chlorotoxin (a 36-amino acid glioblastoma (GBM)-targeting peptide) as an extracellular signaling domain that can bind to glioblastoma cells. These engineered neutrophils were found to exhibit anti-tumor cytotoxicity, stimulate inflammatory cytokine release, and mediate GBM killing via phagocytosis, ROS production and NET formation. CAR-N cells showed antitumor effects superior to such effects using CAR-NK cells, but no combinatorial antitumor effects were found when these cells were combined in vitro or co-administered in vivo.

Disialoganglioside GD2 (GD2) antigen is highly expressed in a variety of pediatric and adult solid tumors, including neuroblastoma, glioma, and melanoma (Saunder et al., 2017, Expert Review of Anticancer Therapy, 17:889-904). GD2 is usually expressed during fetal development, and its expression in normal post-natal tissues is low, usually limited to osteoprogenitors, the brain, peripheral nerves, and skin melanocytes. Based on these characteristics, a number of GD2-specific immunotherapy strategies have been developed, including GD2-specific antibodies, drug coupling, and chimeric antigen receptor-modified T cell therapy (Richman et al., 2018, Cancer Immunology Research 6:36-46; Louis et al., 2011, Blood 118:6050-6056; Straathof et al., 2020, Sci. Transl. Med. 12).

GD2 is expressed in certain solid tumors, including neuroblastoma, retinoblastoma, medulloblastoma, glioblastoma, melanoma, lung cancer, pancreatic cancer bladder cancer, colorectal cancer, sarcoma, or breast cancer. Neuroblastoma, for example, is a malignancy of the sympathetic nervous system, arising from neural crest progenitors that ordinarily develop into sympathetic ganglia and adrenal medulla. Although heterogeneity in clinical presentation and prognosis is a hallmark of tumor cells that highly and selectively express GD2 antigen, anti-GD2 monoclonal antibodies and GD2-CAR T-cells have been used for targeted immunotherapy. Preclinical data have demonstrated that NK T-cells engineered with a GD2-expressing CAR can target tumor cells directly, and indirectly, by destroying tumor-supporting tumor-associated macrophages (TAMs) in neuroblastoma models (Heczey et al., 2021, American Society of Gene and Cell Therapy Annual Meeting; May 11-14; Virtual. Abstract 19). Melanoma is a type of cancer that develops from the pigment-producing cells known as melanocytes. Many melanoma cells express a range of gangliosides including GD2, GM2, GM3 and GD3 that can be a good choice of target for CAR-mediated therapies (Yvon et al., 2009, Cancer Therapy, 15(18):5852-5860).

Thus, there exists a need for new immunotherapies that provide for specific targeting of solid tumors using CAR-weaponized macrophages, neutrophils, natural killer cells and eosinophils which are capable migrating into the solid tumors, killing the tumor cells, and promoting antitumor immune responses.

### SUMMARY OF THE DISCLOSURE

Provided herein are methods for specific targeting of solid tumors using CAR-activated macrophages, natural killer cells, eosinophils, and neutrophils that are capable of migrating into solid tumors, killing the tumor cells, and promoting antitumor immune responses.

Described herein are genetically engineered immune cells that express GD2-CARs with anti-tumor activity that can be used for therapeutic purposes, as well as methods for rapid and scalable production of clinical grade CAR-macrophages (CAR-M), CAR-natural killers (CAR-NK), CAR-eosinophils (CAR-EOS), and CAR-neutrophils (CAR-N) for off-the-shelf immunotherapies.

In one aspect, the disclosure provides a genetically engineered CD11b+ CD14+ macrophage, wherein the CD11b+ CD14+ macrophage expresses an anti-disialoganglioside GD2 (GD2) chimeric antigen receptor (CAR). In some embodiments of the disclosure, the genetically engineered CD11b+ CD14+ macrophage is obtained from a genetically engineered pluripotent stem cell (e.g., an embryonic stem cell or an induced pluripotent stem cell).

In some embodiments of the disclosure, the genetically engineered CD11b+ CD14+ macrophage expresses higher levels of CD80 and lower levels of CD163 and CD206 than a macrophage that does not express an anti-GD2 CAR.

In some embodiments of the disclosure, the genetically engineered CD11b+ CD14+ macrophage exhibits an M1-like anti-cancer phenotype. As provided herein, the genetically engineered CD11b+ CD14+ macrophage is capable of inhibiting tumor cell survival wherein the tumor cell expresses GD2 and the genetically engineered CD11b+ CD14+ macrophage selectively targets cells that express a GD2 antigen. In some embodiments of the disclosure, the tumor cell is a solid tumor (e.g., neuroblastoma, retinoblastoma, medulloblastoma, glioblastoma, melanoma, lung cancer, pancreatic cancer, bladder cancer, colorectal cancer, sarcoma, or breast cancer) and not a blood cancer.

In some embodiments of the disclosure, genetically engineered CD11b+ CD14+ macrophage express an anti-GD2 CAR that has a nucleic acid sequence comprising SEQ ID NO:2 or a sequence having at least 80% sequence identity to SEQ ID NO:2. In certain such embodiments, the genetically engineered CD11b+ CD14+ macrophage expresses an anti-GD2 CAR that comprises a nucleic acid sequence encoding an anti-GD2-scFv1 polypeptide, a hinge polypeptide, a CD28 transmembrane polypeptide, an OX40 polypeptide, and a CD3-zeta polypeptide.

Another aspect of this disclosure provides methods for producing genetically engineered CD11b+ CD14+ macrophage, wherein the CD11b+ CD14+ macrophage expresses an anti-GD2 CAR, wherein the methods comprise: (a) genetically engineering a pluripotent stem cell to express an anti-GD2 chimeric antigen receptor (CAR); (b) culturing the pluripotent stem cell (e.g., an embryonic stem cell or an induced pluripotent stem cell) in a first chemically defined medium for a sufficient time to produce a mesoderm cell; (c) culturing the mesoderm cell seeded at low density in a second chemically defined culture medium that comprises a fibroblast growth factor (FGF) and a vascular endothelial growth factor (VEGF) for a sufficient time to produce a hemogenic endothelial cell; (d) culturing the hemogenic endothelium cell in a third chemically defined culture medium for a sufficient time to produce a CD34+ CD45+ hematopoietic progenitor cells; and (e) culturing the CD34+ CD45+ hematopoietic progenitor cell in a fourth chemically defined culture medium for a sufficient time to produce a CD11b+ CD14+ macrophage.

In some embodiments of this disclosure, the genetically engineered CD11b+ CD14+ macrophage produced by the above methods express higher levels of CD80 and lower levels of CD163 and CD206 than a macrophage that does not express the anti-GD2 CAR.

Yet another aspect of the disclosure provides a pharmaceutical composition comprising genetically engineered CD11b+ CD14+ macrophage that express an anti-GD2 CAR.

Yet another aspect of the disclosure provides methods for producing genetically engineered CD11b+ CD14+ macrophage that express an anti-GD2 CAR, wherein the methods comprise: (a) genetically engineering an hematopoietic progenitor cell (HPC) to express an anti-GD2 chimeric antigen receptor (CAR), wherein the HPC was produced from pluripotent stem cells through arterialized hemogenic endothelium in a low density culture; and (b) culturing the hematopoietic progenitor cell in a feeder-free and serum-free medium for a sufficient time to produce the CD11b+ CD14+ macrophage.

Yet another aspect of this disclosure provides an isolated population of genetically engineered CD11b+ CD14+ macrophages that express an anti-GD2 CAR that are obtained by the above methods. In some embodiments, the isolated population of genetically engineered CD11b+ CD14+ macrophages that express an anti-GD2 CAR comprises about 90% to about 99% CD11b+ CD14+ macrophages.

In another aspect, the disclosure provides genetically engineered CD3⁻ CD56+ natural killer cells, wherein the cells express an anti-disialoganglioside GD2 (GD2) chimeric antigen receptor (CAR) and are capable of inhibiting tumor cell proliferation or survival of cells expressing GD2 antigen.

In some embodiments, the genetically engineered CD3− CD56+ natural killer cell is obtained from a pluripotent stem cell (e.g., an embryonic stem cell or an induced pluripotent stem cell) genetically engineered to express an anti-GD2 CAR, wherein the genetically engineered CD3− CD56+ natural killer cell selectively targets cells that express GD2 antigen.

In some embodiments, the tumor cell is a solid tumor (e.g., neuroblastoma, retinoblastoma, medulloblastoma, glioblastoma, melanoma, lung cancer, pancreatic cancer, bladder cancer, colorectal cancer, sarcoma, or breast cancer) and not a blood cancer.

Another aspect of the disclosure provides methods for producing genetically engineered CD3− CD56+ natural killer cells, wherein the methods comprise: (a) genetically engineering a pluripotent stem cell to express an anti-GD2 CAR; (b) culturing the pluripotent stem cell in a first chemically defined medium for a sufficient time to produce a mesoderm cell; (c) culturing the mesoderm cell seeded at low density in a second chemically defined culture medium that comprises a fibroblast growth factor (FGF) and a vascular endothelial growth factor (VEGF) for a sufficient time to produce a hemogenic endothelial cell; (d) culturing the hemogenic endothelium cell in a third chemically defined culture medium for a sufficient time to produce a CD34+ hematopoietic progenitor cells; and (e) culturing the CD34+ CD45+ hematopoietic progenitor cell in a fourth chemically defined culture medium for a sufficient time to produce a CD3− CD56+ natural killer cell.

Yet another aspect of this disclosure provides methods for producing genetically engineered CD3− CD56+ natural killer cells that express anti-GD2 CAR, wherein the methods comprise: (a) genetically engineering a hematopoietic progenitor cell (HPC) that expresses an anti-GD2 CAR, wherein the HPC was produced from pluripotent stem cells through arterialized hemogenic endothelium in a low-density culture; and (b) culturing the hematopoietic progenitor cell in a feeder-free and serum-free medium for a sufficient time to produce the CD3− CD56+ natural killer cell.

Yet another aspect of this disclosure provides an isolated population of genetically engineered CD3− CD56+ natural killer cells that express an anti-GD2 CAR that are obtained by the above methods. In some embodiments, the isolated population of genetically engineered CD3− CD56+ natural killer cells that express an anti-GD2 CAR comprise about 90% to about 99% CD3− CD56+ natural killer cells.

In another aspect, the disclosure provides genetically engineered EPX+ eosinophils, wherein the EPX+ eosinophils express an anti-disialoganglioside GD2 (GD2) chimeric antigen receptor (CAR) and are capable of inhibiting tumor cell proliferation or survival of cells expressing GD2 antigen. In some embodiments, the genetically engineered EPX+ eosinophil is obtained from a pluripotent stem cell (e.g., an embryonic stem cell or an induced pluripotent stem cell) genetically engineered to express an anti-GD2 CAR and the genetically engineered EPX+ eosinophil selectively targets cells that express GD2 antigen. As disclosed herein, the tumor cell is a solid tumor (e.g., a neuroblastoma cell, a melanoma cell, a glioma cell, a lung cancer cell, a pancreatic cancer cell, or a breast cancer cell) and is not a blood cancer.

Another aspect of the disclosure provides methods for producing genetically engineered EPX+ eosinophils, wherein the methods comprise: (a) genetically engineering a pluripotent stem cell to express an anti-GD2 CAR; (b) culturing the pluripotent stem cell in a first chemically defined medium for a sufficient time to produce a mesoderm cell; (c) culturing the mesoderm cell seeded at low density in a second chemically defined culture medium that comprises a fibroblast growth factor (FGF) and a vascular endothelial growth factor (VEGF) for a sufficient time to produce a hemogenic endothelial cell; (d) culturing the hemogenic endothelium cell in a third chemically defined culture medium for a sufficient time to produce a CD34+ CD45+ hematopoietic progenitor cells; and (e) culturing the CD34+ CD45+ hematopoietic progenitor cell in a feeder-free and serum-free culture medium for a sufficient time to produce a EPX+ eosinophil.

Yet another aspect of this disclosure provides methods for producing genetically engineered EPX+ eosinophils that express anti-GD2 CAR, wherein the methods comprise: (a) genetically engineering a hematopoietic progenitor cell (HPC) that expresses an anti-GD2 CAR, wherein the HPC was produced from pluripotent stem cells through arterialized hemogenic endothelium in a low-density culture; and (b) culturing the hematopoietic progenitor cell in a feeder-free and serum-free medium for a sufficient time to produce the EPX+ eosinophil.

Yet another aspect of this disclosure provides an isolated population of genetically engineered EPX+ eosinophils that express an anti-GD2 CAR that are obtained by the above methods. In some embodiments, the isolated population of genetically engineered EPX+ eosinophils that express an anti-GD2 CAR comprise about 30% to about 40% EPX+ eosinophils. In some embodiments, the isolated population of genetically engineered EPX+ eosinophils that express an anti-GD2 CAR can be further purified to about 90% to about 99% EPX+ eosinophils.

Yet another aspect of the disclosure provides a genetically engineered CD11b+ CD15+ neutrophil, wherein the CD11b+ CD15+ neutrophil expresses an anti-disialoganglioside GD2 (GD2) chimeric antigen receptor (CAR). In some embodiments of the disclosure, the neutrophil is produced from a genetically engineered pluripotent stem cell (e.g., an embryonic stem cell or an induced pluripotent stem cell). In some embodiments of this disclosure, the genetically engineered CD11b+ CD15+ neutrophil selectively targets cells that express GD2 antigen. In some embodiments of the disclosure, the genetically engineered CD11b+ CD15+ neutrophil is capable of killing solid tumor cells (e.g., cell is a neuroblastoma cell, a melanoma cell, a glioma cell, a sarcoma cell, a lung cancer cell, a breast cancer cell, or a pancreatic cancer cell) that expresses GD2 and is not a blood cancer.

Another aspect of this disclosure provides methods for producing genetically engineered CD11b+ CD15+ neutrophils that express an anti-GD2 CAR, wherein the methods comprise: (a) genetically engineering a pluripotent stem cell (PSC) to express an anti-GD2 chimeric antigen receptor (CAR); (b) introducing exogenous ETV2 in the genetically engineered PSC and culturing the ETV2-induced PSC in a xenogen-free, feeder-free, and serum-free medium to produce a population of ETV2-induced endothelial progenitor cells; (c) culturing the ETV2-induced endothelial progenitor cells in xenogen-free, feeder-free, and serum-free medium comprising for a sufficient time to produce non-adherent myeloid progenitors; and (d) culturing the myeloid progenitors in xenogen-free, feeder-free, and serum-free medium for a sufficient time to differentiate the non-adherent myeloid progenitors into CD11b+ CD15+ neutrophils In some embodiments, the myeloid progenitors express CD34 and CD45 by day 9 in culture.

Yet another aspect of this disclosure provides an isolated population of genetically engineered CD11b+ CD15+ neutrophils that express an anti-GD2 CAR that are produced by the above methods. In some embodiments, the isolated population of genetically engineered CD11b+ CD15+ neutrophils that express an anti-GD2 CAR comprises about 90% to about 99% CD11b+ CD15+ neutrophils.

Yet another aspect of this disclosure provides a pharmaceutical composition comprising the genetically engineered CD11b+ CD14+ macrophage, the genetically engineered CD3− CD56+ natural killer cell, the genetically engineered EPX+ eosinophil, the genetically engineered CD11b+ CD15+ neutrophil that are obtained by the above methods, or any combination of them thereof.

Yet another aspect of this disclosure provides a method of treating a solid tumor in a subject in need thereof, the method comprising administering a therapeutically effective amount of the pharmaceutical composition comprising the genetically engineered CD11b+ CD14+ macrophage, the genetically engineered CD3− CD56+ natural killer cell, the genetically engineered EPX+ eosinophil, the genetically engineered CD11b+ CD15+ neutrophil, or any combination of them thereof.

Yet another aspect of this disclosure provides methods for reducing proliferation of a solid tumor cell, wherein the methods comprise contacting the solid tumor with the genetically engineered CD11b+ CD14+ macrophage, the genetically engineered CD3− CD56+ natural killer cell, the genetically engineered EPX+ eosinophil, the genetically engineered CD11b+ CD15+ neutrophil, or any combination of them thereof.

As disclosed herein, one or more doses of the genetically engineered CD11b+ CD14+ macrophage, the genetically engineered CD3− CD56+ natural killer cell, the genetically engineered EPX+ eosinophil, and the genetically engineered CD11b+ CD15+ neutrophil, or any combination thereof, can be used.

As disclosed herein, the genetically engineered EPX+ eosinophil promotes the anti-tumor activity of the genetically engineered CD3− CD56+ natural killer cell.

As disclosed herein, the genetically engineered CD11b+ CD14+ macrophage promotes the anti-tumor activity of the genetically engineered CD3− CD56+ natural killer cell.

As disclosed herein, the genetically engineered CD11b+ CD15+ neutrophil secretes inflammatory cytokines after co-culture with tumor cells expressing GD2.

As disclosed herein, solid tumors treated by the pharmaceutical compositions provided herein include but are not limited to neuroblastoma, retinoblastoma, medulloblastoma, glioblastoma, melanoma, lung cancer, pancreatic cancer, bladder cancer, colorectal cancer, sarcoma, or breast cancer. As disclosed herein, the solid tumor expresses GD2. As disclosed herein, the genetically engineered CD11b+ CD14+ macrophage, the genetically engineered CD3− CD56+ natural killer cell, the genetically engineered EPX+ eosinophil, the genetically engineered CD11b+ CD15+ neutrophil are autologous or allogeneic to the subject.

In some embodiments, the genetically engineered CD11b+ CD14+ macrophage expresses an anti-disialoganglioside GD2 (GD2) chimeric antigen receptor (CAR) and also has inhibited expression of signal regulatory protein alpha (SIRPa). The expression of SIRPa is inhibited by gene mutation, RNA-meditated inhibition, RNA editing, DNA gene editing or base editing. In some embodiments, the expression of SIRPa is knocked out by gene editing method, wherein the gene editing method involves using a nuclease selected from a meganucleases, ZGNs, TALENS, and CAS enzyme.

These and other features, objects, and advantages of this invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

The disclosure will be better understood and features, aspects, and advantages other than those set forth above will become apparent when consideration is given to the following detailed description thereof. Such detailed description refers to the following drawings.

FIG. 1A are schematic diagrams of arterial endothelial cell (AECs) and hematopoietic cell differentiation. High cell density (HD), 0.5-2.0×10⁵ cells/cm²; low cell density (LD), 0.2-2×10⁴ cells/cm². High density (HD) cells were treated with or without 50 ng/ml BMP4 from day 2-6 with five factor medium. Low density (LD) cells were treated with or without LDN (a BMP signaling inhibitor) from day 2-6 with five factor media. FIG. 1B shows statistics of total hematopoietic cells generated from 1 well of 12-well plate at day 10.

FIG. 2 is a schematic representation of the 5′-MCS-3′β-globin construct for ETV2 modified mRNA (mmRNA) synthesis. MCS is an abbreviation for multiple cloning sites.

FIG. 3 is a schematic diagram of optimized protocol for generating wild-type and CAR-GD2 neutrophils in defined serum free and feeder free conditions.

FIG. 4A is a schematic diagram of macrophage differentiation. FIG. 4B shows flow cytometry analysis of CD14 and CD11b expression at day 20. FIG. 4C is a graph showing statistics data for the macrophage population. FIG. 4D shows flow cytometry analysis of CD68 and SIRPa/CD172A expression at day 20 of differentiation. FIG. 4E shows cell morphology at day 20 of differentiation. FIG. 4F shows Wright-Giemsa staining of cytospins from day 20 differentiation. FIG. 4G is a graph showing total yield of macrophage from one starting human pluripotent stem cell. FIG. 4H shows phagocytosis of yeast particles by macrophages. Zymosan An S. cerevisiae BioParticles (Texas Red conjugate; Life Technologies) were prepared in phosphate-buffered saline (PBS; 10 mg/mL=2×10⁹ particle/mL). 20 μL particles were added to 2 mL media containing 4×10⁵ macrophage. Phagocytosis was imaged over time.

FIG. 5A is a graph showing phagocytosis of cancer cells. CHLA-20-AkaLuc2-eGFP neuroblastoma cells were mono-cultured or co-cultured with macrophage for 20-24 hours. Statistics of CHLA-20 cell survival are shown. FIG. 5B is a schematic diagram of GD2-CAR constructs.

FIG. 5C shows junctional PCR analysis of AAVS1-CARb knock in allele and WT AAVS1 allele to demonstrate correct CAR integration. WT cells (without gene editing) are used as a control. FIG. 5D shows qPCR analysis of AAVS1-GD2-CARb-PuroR copy number. FIG. 5E shows karyotyping of CARb hPSC line.

FIG. 5F shows flow cytometry analysis of CD14 and CD11b expression at day 25 of differentiation. FIG. 5G shows Wright-Giemsa staining of WT-M and CARb-M cytospins. FIG. 5H is a graph showing RT-qPCR analysis of GD2-CARa, GD2-CAR1, and GD2-CARb expression in macrophages. Construct-specific primers were used for each cell line. FIG. 5I is a RT-qPCR analysis of CARb expression compared to WT-M. FIG. 5J demonstrates anti-GD2 CARb expression in macrophages using immunofluorescence staining with antibody against GD2 antibody 14G2a.

FIG. 6A are graphs showing in vitro cytotoxicity assay results. AAVS1-AkaLuc-eGFP labeled CHLA-20 cells were mono-cultured or co-cultured with macrophage for 20-24 hours. A luminescence assay was used to measure CHLA-20 cell survival. FIG. 6B shows tumor killing in these experiments. AAVS1-AkaLuc-eGFP labeled CHLA-20 cells were mono-cultured or co-cultured with macrophage for 20-24 hours. CHLA-20 cells were labeled by GFP.

FIG. 7A shows flow cytometry analysis M1/M2 markers. FIG. 7B are graphs showing statistics of M1 markers (left) and M2 markers (right). MFI: mean fluorescent intensity. *:p<0.05.

FIG. 7C shows flow cytometry analysis of M1/M2 markers in WT-M and CARb-M with or without treatment. FIG. 7D shows statistics of M1/M2 markers. Data are presented as mean±SD. Student's test, *p<0.05. CARb-Ms are generated from 2 different clones.

FIG. 8A is a flow cytometry analysis of GD2 expression in different cells. CAR-M is CARb-M, AEC are arterial endothelial cell, and SMC are smooth muscle cells. FIG. 8B is a bar graph showing in vitro cytotoxicity with GD2 negative cells. AEC, SMC, K562, Raji, and CHLA-20 were co-cultured with macrophages (E:T=3:1) for 20 h. AEC and SMC were derived from H1 ES cells and labeled with NanoLuc. K562, Raji, and CHLA-20 cells were labeled by AkaLuc-eGFP. GD2-CARb macrophages were derived from H9 ES cells.

FIG. 9A through FIG. 9C show the results of genetic ontology (GO) term analysis of RNA-seq data. FIG. 9A shows M1-related GO terms enriched in CAR-macrophages. FIG. 9B shows M1-related GO terms enriched in GD2-CAR macrophages co-cultured with CHLA-20 cells. Macrophages were sorted for RNA-seq. FIG. 9C shows other interested GO terms enriched in CAR-macrophages co-cultured with CHLA-20 cells.

FIG. 9D is a heatmap showing M1-related genes in macrophages co-cultured with CHLA-20.

FIG. 10A is a graph showing cell survival of CHLA-20-AkaLuc-eGFP neuroblastoma cells exposed to WT macrophages and CAR-macrophages in a macrophage: cancer cell ratio was shown in 6:1, 3:1, 1:1 and 0:1. FIG. 10B is a graph showing cell survival of WM266-4 melanoma cells exposed to WT macrophages and GD2-CAR macrophages in a macrophage: cancer cell ratio was shown in 6:1, 3:1, 1:1 and 0:1. FIG. 10C show flow cytometry plots of CHLA-20 cells co-cultured with WT-MS and CARb-Ms. CHLA-20 cells are labeled by GFP. WT-Ms and CARb-Ms are labeled by SIRPa immunostaining. FIG. 10D are photomicrographic images showing phagocytosis of CHLA-20 cells by CARb-Ms. CHLA-20-AkaLuc-GFP cells were co-cultured with macrophages for 6 h (E:T=3:1). Green arrows indicate CHLA-20 cells, red arrows indicate WT-Ms, and yellow arrows indicate phagocytosis of CARb-Ms. FIG. 10E shows killing of CHLA-20 neuroblastoma cells by CARb-Ms with or without treatment. CHLA-20-AkaLuc-GFP cells were mono-cultured or co-cultured with macrophages at E:T ratio=3:1 for 20-24 h.

FIG. 11 shows secretome analysis of macrophages. WT-Ms and CARb-Ms were mono-cultured or co-cultured with CHLA-20 for 20 h. Cell culture media were collected for secretome analysis.

FIG. 12A is a schematic diagram illustrating mouse model experiments. CHLA-20-AkaLuc-GFP cells were injected subcutaneously into mice alone or with WT-Ms or CARb-Ms. Luminescent signals were measured at 1, 8 15, 22, and 29 days post injection. FIG. 12B is a quantification of tumor burden as shown by luminescent signals. FIG. 12C shows body weight of the animals tested.

FIG. 12D shows tumor burden assessed by bioluminescent imaging at indicated time points.

FIG. 13A is a schematic diagram of NK cell differentiation. FIG. 13B shows flow cytometry analysis of CD3 and CD56 expression of NK cells derived from wild-type and GD2-CAR iPS cells. FIG. 13C shows flow cytometry analysis of GD2-CAR expression of NK cells. FIG. 13D is a schematic diagram of eosinophil (EOS) differentiation. FIG. 13E shows flow cytometry analysis of EPX expression of EOS derived from GD2-CAR iPS cells.

FIG. 14A and FIG. 14B show the anti-tumor effects of CAR-M and CAR-NK combinations. FIG. 14A shows killing of CHLA-20 neuroblastoma cells by GD2-CAR-M and GD2-CAR-NK. CHLA20-AkaLuc-GFP cells were mono-cultured or co-cultured with macrophages and NK cells at different effector:target (E:T) ratios for 20-24 hours. Medium: 50% DM5 with 50% NKM medium (Table 2). Antitumor activity was illustrated by cancer cell survival that was measured by luciferase assay. FIG. 14B shows killing of WM266-4 melanoma cells by GD2-CAR-M and GD2-CAR-NK. WM266-4-AkaLuc-GFP cells were mono-cultured or co-cultured with macrophages and NK cells at different effector:target (E:T) ratios for 20-24 hours. Medium: 50% DM5 with 50% NKM. Antitumor activity was illustrated by cancer cell survival that was measured by luciferase assay.

FIG. 15A shows imaging of WM266-4 melanoma cell survival in cultures. Two days before the co-culture experiment, AAVS1-AkaLuc-eGFP labeled melanoma cell were cultured in U bottom plate to form spheroids. GD2-CAR-M and/or GD2-CAR-NK cells were added and co-cultured for another 2 days. Images were taken daily. ×2 indicates double amounts of cells. FIG. 15B is a bar graph wherein WM266-4 melanoma cell survival was measured by luciferase assay. Statistics of cell survival results for FIG. 15A are represented as mean±SD. ×2, double amounts of cells.

FIG. 16A show the results of flow cytometry analysis of granzyme B (GZB), perforin, and CD107a expression on CD11b labelled macrophages and CD56 labelled NK cells.

FIG. 16B are bar graphs showing statistics of granzyme B (GZB), perforin, and CD107a, the results are represented as mean±SD.

FIG. 17 shows imaging of WM266-4 cell survival and GD2-CAR-NK in co-culture with GD2-CAR-M. Two days before the co-culture experiment, AAVS1-AkaLuc-eGFP labeled melanoma cell were cultured in U bottom plate to form spheroids. CAR-M and/or CAR-NK cells were added and co-cultured for another day. Many CAR-NK were still in the medium when CAR-NK was added to the spheroid alone, but most of CAR-NK was attached to the spheroid when CAR-M was also added.

FIG. 18A are representative photomicrographic images showing phagocytosis of WM266-4 cells by CAR-M. WM266-4-AkaLuc-GFP cells were co-cultured with macrophage and/or NK cells for 6 hours (E:T=4:1). Yellow arrows indicate phagocytosis of WM266-4 cells by CAR-M. FIG. 18B are representative flow cytometry plots showing WM266-4 cells co-cultured with CAR-M and/or CAR-NK. WM266-4 cells are labeled by GFP. CAR-M are labeled by SIRPa immunostaining.

FIG. 19 are bar graphs showing secretome analysis. WM266-4 was mono-cultured or co-cultured with CAR-M and/or CAR-NK for 20 hours. Cell culture media were collected for secretome analysis.

FIG. 20 is a schematic diagram showing combinations of CAR-M and WT-NK promotes antitumor activity, indicating that CAR-M might be able to activate recipient immune cells (such as recipient NK cells).

FIG. 21A and FIG. 21B show combinational effects between WT-M and WT-NK, CAR-M and WT-NK. Antitumor activity was illustrated by cancer cell survival that was measured by luciferase assay. FIG. 21A shows that CAR-M promote anti-tumor activity of WT-NK and CAR-NK. FIG. 21B is a bar graph showing the relative number of cancer cells remaining after different treatments, wherein CAR-M alone did not reduce melanoma cells, and CAR-M promoted anti-tumor activity of WT-NK (CAR-M+WT-NK) or CAR-NK (CAR-M+CAR-NK).

FIG. 22 shows NK cell proliferation measured by carboxyfluorescein succinimidyl ester (CFSE) staining. Both WT-M and CAR-M promoted NK cell proliferation in the presence of tumor cells.

FIG. 23A and FIG. 23B show the anti-tumor effects of CAR-EOS and CAR-NK combinations. FIG. 23A shows killing of CHLA-20 neuroblastoma by GD2-CAR-EOS and GD2-CAR-NK. CHLA20-AkaLuc-GFP or WM266-4-AkaLuc-GFP cells were mono-cultured or co-cultured with EOS and NK cells at different effector:target (E:T) ratios for 20-24 hours. Medium: 50% EM with 50% NKM. Antitumor activity was illustrated by cancer cell survival that was measured by luciferase assay. FIG. 23B shows killing of WM2666-4 melanoma cells by GD2-CAR-EOS and GD2-CAR-NK. CHLA20-AkaLuc-GFP or WM266-4-AkaLuc-GFP cells were mono-cultured or co-cultured with EOS and NK cells at different effector:target (E:T) ratios for 20-24 hours. Medium: 50% EM with 50% NKM. Antitumor activity was illustrated by cancer cell survival that was measured by luciferase assay.

FIG. 24A and FIG. 24B show results that a combination of CAR-M and CAR-NK improve antitumor activity in vivo. 2×10⁵ WM266-4 cells were injected to the hind flank of mice. Three days later, 2×10⁶ CAR-M and/or CAR-NK cells were administered to the mice by intravenous injection. For CAR-NK×2, 4×10⁶ CAR-NK were administered by intravenous injection. Luminescent signals were measured at 0, 7, and 14 days post CAR-M/NK injection, using 5-6 mice per group. FIG. 24A is a schematic diagram of this experimental protocol. FIG. 24B shows graphically the results on days 0, 7 and 14.

FIG. 25 is a schematic diagram of a GD2-CAR construct and position of probes used for Southern blot analysis.

FIG. 26 is a Southern blot analysis of GD2-CAR clones established from BM9 iPSC line. Clones with correct insertion are circled.

FIG. 27 shows representative phase contrast microscopic images illustrating morphology differences during hematoendothelial development and neutrophil differentiation following transduction of wild-type and CAR-GD2 hPSCs with ETV2 mmRNA.

FIGS. 28A and 28B are representative microscopic images of Wright staining showing the morphology of WT in low (top) and high (bottom) magnifications (FIG. 28A) and GD2-CAR in low (top) and high (bottom) magnifications (FIG. 28B) neutrophils.

FIG. 29A through FIG. 29B show flow cytometric analysis of CD45, CD11b, CD15, CD66b, CD95, CD54, and CD182 expression in WT and GD2-CAR generated neutrophils.

FIG. 29C shows GD2-CAR RNA expression in neutrophils.

FIG. 30A through FIG. 30D are graphs of in vitro cytotoxicity assay of neutrophils generated from ETV2 mmRNA transfected wild-type and CAR-GD2 hPSCs. Percentages of cell lysis, when neutrophils were co-cultured with GD2-positive and GD2-negative tumor cells is shown by E:T (effector:target) ratio for WM266-4-Luc2-eGFP GD2-positive tumor cells (FIG. 30A), CHLA-20-AkaLuc-eGFP GD2-positive tumor cells (FIG. 30B), SKBR3-Luc2-eGFP GD2-negative tumor cells (FIG. 30C), and SKOV3-Luc2-eGFP GD2-negative tumor cells (FIG. 30D).

FIG. 30E through FIG. 30F show that neutrophils secrete inflammatory cytokines after co-culture with GD2 positive tumor cells. Neutrophils generated from ETV2 mmRNA transfected unmodified hiPSCs (WT-N) and GD2-CAR hiPSCs (CAR-N), were either cultured alone or co-cultured with GD2 positive tumors (E:T=1-0:1) for 12 hours and supernatants were collected for assessment using a Human Inflammation 20-Plex Procarta Plex Panel. FIG. 30E shows heatmaps depicting expression of 29 different cytokines in all groups of samples and FIG. 30F shows graphs depicting level of selected cytokines in supernatants (pg/ml) of all samples.

FIG. 31 is a schematic diagram of an in vivo cytotoxicity assay with CAR-GD2 neutrophils in a mouse melanoma xenograft model. Mice were inoculated intraperitoneally with 3×10⁵ Luc2-eGFP-expressing WM-266-4 melanoma cells. Melanoma cell engraftment was assessed by IVIS imaging (Perkin Elmer) 3 days later for baseline pretreatment reading. On day 4 after melanoma injection, mice were left untreated, or treated with 10⁷ unmodified neutrophils or CAR-GD2 neutrophils injected intraperitoneally every 7 days. Tumor burden was determined by bioluminescent imaging.

FIG. 32A through FIG. 32E show the results of animal experiments using melanoma cells injected into mice and the effects of CAR-GD2 neutrophils. FIG. 32A and FIG. 32B are images taken at days 3, 7, 14, 21, and 28 of the NCG (FIG. 32A) and NSG (FIG. 32B) mice that were un-injected, mice that were injected with WT neutrophils and WM-266-4-Luc2-eGFP melanoma cells, mice that were injected with CAR-GD2 neutrophils and WM-266-4-Luc2-eGFP melanoma cells, and mice that were injected with only WM-266-4-Luc2-eGFP melanoma cells. FIG. 32C is a graph showing the total flux at days 3, 7, 14, 21, and 28 of mice that were un-injected, mice that were injected with WT neutrophils and melanoma cells, mice that were injected with CAR-GD2 neutrophils and melanoma cells, and mice that were injected with only melanoma cells. The difference in total flux between WT neutrophils and CAR-GD2 neutrophils was statistically significant by analysis of variance (p<0.0001). FIG. 32D shows a Kaplan-Meier curve representing the percent survival of the experimental groups: Tumor only, or treated with WT Neutrophils, CAR-GD2 Neutrophils and negative control. Significant differences in survival were observed between WT Neutrophils and CAR-GD2Neutrophils by Mantel-Cox test (P<0.001). FIG. 32E shows weekly body weights over 30 days (n=8) between WT Neutrophils and CAR-GD2Neutrophils by analysis of variance (p<0.0001).

FIG. 33 is a schematic diagram of an in vivo cytotoxicity assay with CAR-GD2 neutrophils in a mouse subcutaneous melanoma xenograft model. Mice were injected under the skin into the right flank with 3×105 Luc2-eGFP-expressing WM-266-4 melanoma cells. After establishing a palpable subcutaneous tumor 15 days after melanoma injection, mice were left untreated, or treated with 10⁷ unmodified neutrophils or CAR-neutrophils as indicated. Tumor burden was determined by bioluminescent imaging or using caliper.

FIG. 34A through FIG. 34E show the results of animal experiments using mice with subcutaneous melanoma cells treated withCAR-GD2 neutrophils. FIG. 34A bioluminescent images are taken at days 14, 21, 28 and 35 after melanoma injection in mice that were injected with WT neutrophils and WM-266-4-Luc2-eGFP melanoma cells, mice that were injected with CAR-GD2 neutrophils and WM-266-4-Luc2-eGFP melanoma cells, and mice that were injected with only WM-266-4-Luc2-eGFP melanoma cells. FIG. 34B is a graph showing the total flux at days 14, 21, 28 and 35 of mice that were uninjected, mice that were injected with WT neutrophils and melanoma cells, mice that were injected with GD2-CAR neutrophils and melanoma cells, and mice that were injected with only melanoma cells. The differences in total flux between CAR-GD2 neutrophils and WT neutrophils or tumor only were statistically significant by analysis of variance (p=0.00025 and p=0.00014 correspondingly). FIG. 34C is a graph showing the intravital tumor volume measured by caliper at days 14, 21, 28 and 35 of mice that were injected with WT neutrophils and melanoma cells, mice that were injected with GD2-CAR neutrophils and melanoma cells, and mice that were injected with only melanoma cells. The differences in tumor volume between CAR-GD2 neutrophils and WT neutrophils was statistically significant by analysis of variance (p=0.004). FIG. 34D is a graph showing the tumor volume measured by caliper at time of death of mice that were injected with WT neutrophils and melanoma cells, mice that were injected with CAR-GD2 neutrophils and melanoma cells, and mice that were injected with only melanoma cells. The differences in tumor volume between CAR-GD2 neutrophils and WT neutrophils or tumor only were statistically significant by analysis of variance (p<0.00053 and p<0.00045 correspondingly). FIG. 34E shows a Kaplan-Meier curve representing the percent survival of the experimental groups: Tumor only, or treated with WT Neutrophils, CAR-GD2 Neutrophils and negative control. Significant differences in survival were observed between WT Neutrophils and CAR-GD2 Neutrophils by Mantel-Cox test (P<0.001).

FIG. 35A is a schematic diagram of intraperitoneal injection of Cellvue Burgundy-labelled neutrophils generated from ETV2 mmRNA transfected unmodified hiPSCs (WT Neutrophils) and GD2-CAR hiPSCs (CAR-GD2 Neutrophils) for in vivo cell tracking study. FIG. 35B shows time-dependent biodistributions of neutrophils in whole body determined by fluorescence imaging at indicated hours.

FIG. 36A is a schematic diagram of experiment. Mice were injected under the skin into the right and left flank with 5×10⁵ Luc2-eGFP-expressing WM-266-4 melanoma cells. After establishing a palpable subcutaneous tumor 21 days after melanoma injection, mice were injected with 2×10⁷ unmodified neutrophils (WT) or CAR-neutrophils (CAR-GD2). 24 hours after neutrophil injected, fluorescence and bioluminescence of whole body and isolated organs and tumors was evaluated. FIG. 36B shows biodistributions of neutrophils in whole body determined by fluorescence imaging at 24 hours after neutrophil injection. FIG. 36C shows fluorescence and bioluminescence of organs isolated from mice that were uninjected and mice that were injected with wild type or CAR-GD2 neutrophils 24 hours before. FIG. 36D shows fluorescence and bioluminescence of subcutaneous tumors isolated from mice that were injected with wild type or CAR-GD2 neutrophils 24 hours before mice were sacrificed.

FIG. 37 is a schematic of CRISPR/Cas9 driven knockout of signal regulatory protein alpha (SIRPa) gene at exon 3 using two sgRNAs.

FIG. 38A shows a schematic of SIRPa knockout strategy. Exon 2 or exon 3 was deleted by CRISPR-Cas9. FIG. 38B is the genotyping results showing the knockout of exon 2 and 3. FIG. 38C demonstrates killing of CHLA-20 neuroblastoma and WM266-4 melanoma cells by anti-GD2-CAR-M. CAR-SIRPa-E2/E3: exon 2 or 3 of SIRPa was deleted in the CAR-M.

### DETAILED DESCRIPTION OF THE DISCLOSURE

This disclosure is based, at least in part, on the inventors' production of GD2-CAR macrophages, GD2-CAR neutrophils, GD2-CAR natural killer cells, and GD2-CAR eosinophils that have anti-tumor activity, methods of generating CAR macrophages, CAR neutrophils, CAR natural killer cells, and CAR eosinophils from pluripotent stem cells, and methods of treating cancers that express GD2 using the GD2-CAR macrophages, GD2-CAR neutrophils, GD2-CAR natural killer cells and GD2-CAR eosinophils described herein and therapeutically effective combinations thereof.

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

The term “contacting” includes the physical contact of at least one substance to another substance.

As used herein, “treatment” refers to the clinical intervention made in response to a disease, disorder, or physiological condition of the subject or to which a subject can be susceptible (e.g., a tumor that expresses GD2). The aim of treatment includes the alleviation or prevention of symptoms, slowing or stopping the progression or worsening of a disease, disorder, or condition and/or the remission of the disease, disorder, or condition.

The terms “effective amount” or “therapeutically effective amount” refer to an amount sufficient to effect beneficial or desirable biological and/or clinical results. In other words, a “therapeutically effective” amount is an amount that will provide some alleviation, mitigation, or decrease in at least one clinical symptom in the subject.

The terms “express” or “expression” refer to transcription and translation of a nucleic acid coding sequence resulting in production of the encoded polypeptide. “Express” or “expression” also refers to antigens such as GD2 that are expressed on cell surfaces.

As used herein, the term “subject” refers to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The subject can be a human patient that is at risk for, or suffering from, a tumor that expresses GD2 (e.g., neuroblastoma or melanoma). The subject can also be a human that is at risk for, or suffering from, a tumor that expresses GD2. The human subject can be of any age (e.g., an infant, child, or adult).

The term “anti-tumor” refers to the reduction in size of solid tumor, inhibition of tumor growth, or increase in tumor cell death.

### Chimeric Antigen Receptor (CAR)

This disclosure provides chimeric antigen receptors (CARs) that can bind to an antigen of interest. The term “chimeric antigen receptor (CAR)” refers to a recombinant fusion protein that has an antigen-specific extracellular domain coupled to an intracellular domain that directs the cell to perform a specialized function upon binding of an antigen to the extracellular domain. In some embodiments, a CAR comprises an antigen-specific extracellular domain (e.g., a single chain variable fragment, scFV, that can bind a surface-expressed antigen of a malignancy, such as GD2) coupled to an intracellular domain (e.g., CD28, CD137, ICOS, CD27, 4-1BB, OX40, CD40L, or CD3z, FcRg) by a transmembrane domain (e.g., derived from a CD4, CD8a, CD28, IgG or CDS-z transmembrane domain).

The antigen-specific extracellular domain of a CAR can recognize and specifically bind an antigen, typically a surface-expressed antigen of a malignancy (e.g., GD2).

An antigen-specific extracellular domain suitable for use in a CAR can be any antigen binding polypeptide, one or more scFv (e.g., anti-GD2 scFv1), or another antibody-based recognition domain (cAb VHH, camelid antibody variable domains) or humanized versions thereof, IgNAR VH (shark antibody variable domains) and humanized versions thereof, sdAb VH (single domain antibody variable domains) and “camelized” antibody variable domains are also suitable for use. In some instances, T cell receptor (TCR)-based recognition domains such as single chain TCR can be used as well as ligands for cytokine receptors.

In certain embodiments, the CAR binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-a, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), K-light chain, kinase insert domain receptor (KDR), Lewis Y (LeY), LI cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MARTI, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A and ERBB.

The antigen-specific extracellular domain can be linked to the intracellular domain of the CAR by a transmembrane domain, e.g., derived from a CD4, CD8a, CD28, IgG or 03-z transmembrane domain. The transmembrane domain traverses the cell membrane, anchors the CAR to the cell surface, and connects the extracellular domain to the intracellular signaling domain, thus impacting expression of the CAR on the cell surface. CARs can also further comprise one or more costimulatory domain and/or one or more spacer. A costimulatory domain can be derived from the intracellular signaling domains of costimulatory proteins that enhance cytokine production, proliferation, cytotoxicity, and/or persistence in vivo. A hinge domain connects (i) the antigen-specific extracellular domain to the transmembrane domain, (ii) the transmembrane domain to a costimulatory domain, (iii) a costimulatory domain to the intracellular domain, and/or (iv) the transmembrane domain to the intracellular domain. For example, inclusion of a hinge domain (e.g., IgG1, IgG2, IgG4, CD28, CD8) between the antigen-specific extracellular domain and the transmembrane domain can affect flexibility of the antigen-binding domain and thereby CAR function. Suitable transmembrane domains, costimulatory domains, and spacers are known in the art.

### GD2-CAR Constructs

Disialoganglioside GD2 (GD2) (C47H134N4032) is a disialoganglioside belonging to b-series gangliosides. It comprises five monosaccharides linked to ceramide, having a carbohydrate sequence of GalNAcβ1-4(NeuAcα2-8NeuAcα2-3)Galβ1-4Glcβ1-1. GD2 is expressed almost exclusively on tumors, including but not limited to, neuroblastoma, melanoma, glioma, sarcoma (e.g., soft tissue sarcoma tumor cells), lung cancer, breast cancer stem cells, and pancreatic cancer. GD2 is not expressed (or is expressed at low levels) on normal tissues and the expression of GD2 in normal cells is restricted to the brain, peripheral pain fibers, and skin melanocytes. In contrast, expression of GD2 on primary neuroblastomas can be, for example, about 10⁷ molecules per cell.

The terms “GD2-CAR” or “anti-GD2 CAR” or “CAR” are used interchangeably herein and refer to a CAR construct that comprises an extracellular domain that specifically recognizes GD2.

The GD2-CAR constructs of this disclosure can comprise the CARa, CARb, or CAR1 nucleic acid sequences.

In some embodiments, the GD2-CAR constructs of this disclosure can comprise a nucleic acid sequence encoding an anti-GD2-scFv1 polypeptide, a Myc tag polypeptide, a CD8a hinge polypeptide, a CD8a transmembrane polypeptide, a CD137 polypeptide, and a CD3-zeta polypeptide (e.g., CARa construct). In some embodiments, these components are present on the CAR construct in the 5′ to 3′ direction.

In some embodiments, the GD2-CAR constructs of this disclosure can comprise a nucleic acid sequence encoding an anti-GD2-scFv1 polypeptide, a CD8a hinge polypeptide, a CD8a transmembrane polypeptide, a CD28 cytoplasmic polypeptide, a CD137 polypeptide, and a CD3-zeta polypeptide (e.g., CAR1 construct). In some embodiments, these components are present on the CAR construct the 5′ to 3′ direction.

In some embodiments, the GD2-CAR constructs of this disclosure can comprise a nucleic acid sequence encoding an anti-GD2-scFv1 polypeptide, a hinge polypeptide, a CD28 transmembrane polypeptide, an OX40 polypeptide, and a CD3-zeta polypeptide (e.g., CARb construct). In some embodiments, these components are present on the CAR construct the 5′ to 3′ direction.

The extracellular domain of the CAR constructs (e.g., anti-GD2-scFv1 polypeptide, hinge polypeptide, MYC-tag) can be of varying lengths. Likewise, the intracellular domain of the CAR constructs (e.g., CD137, CD3z, CD28-cytoplasmic, OX40) can be of varying lengths.

The GD2-CAR constructs of this disclosure can comprise a nucleic acid sequence as set forth in any of SEQ ID NOS:1-3, or can have at least 50%, 60%, 70%, 80%, 85%, 86%, 87%, 88%, 89%, 90%, 91%, 92%, 93%, 94%, 95%, 96%, 97%, 98%, or 99% sequence identity to the nucleic acid sequence set forth in any of SEQ ID NOS:1-3. In some embodiments, the GD2-CAR construct comprises the nucleic acid sequence as set forth in SEQ ID NO: 2 or a sequence having at least 80% sequence identity to the sequence set forth in SEQ ID NO:2.

In some embodiments, the anti-GD2 ScFv1 nucleic acid sequence can comprise the sequence of nucleic acids starting at position 64 and ending at position 780 of the nucleic acid sequence of SEQ ID NO:1 or the sequence of nucleic acids starting at position 1 and ending at position 855 of the nucleic acid sequences of SEQ ID NO:2 or SEQ ID NO:3.

The GD2-CAR constructs of this disclosure can comprise an amino acid sequence as set forth in any of SEQ ID NOS:5-7 or can have at least 50%, 60%, 70%, 80%, 85%, 86%, 87%, 88%, 89%, 90%, 91%, 92%, 93%, 94%, 95%, 96%, 97%, 98%, or 99% sequence identity to the amino acid sequence set forth in any of SEQ ID NOS:5-7. In some embodiments, the GD2-CAR construct comprises the amino acid sequence as set forth in SEQ ID NO: 5 or a sequence having at least 80% sequence identity to the sequence set forth in SEQ ID NO:5.

The term “construct” refers to an artificially designed segment of DNA that can be used to incorporate genetic material into a target cell (e.g., an hPSC).

The term “sequence identity” refers to the number of identical or similar nucleotide bases on a comparison between a test and reference oligonucleotide or nucleotide sequence. Sequence identity can be determined by sequence alignment of a first nucleic acid sequence to identify regions of similarity or identity to second nucleic acid sequence. As described herein, sequence identity is generally determined by alignment to identify identical residues. Matches, mismatches, and gaps can be identified between compared sequences by techniques known in the art. Alternatively, sequence identity can be determined without taking into account gaps as the number of identical positions/length of the total aligned sequence×100. In one embodiment, the term “at least 90% sequence identity to” refers to percent identities from 90 to 100%, relative to the reference nucleotide sequence. Identity at a level of 90% or more is indicative of the fact that, assuming for exemplary purposes a test and reference polynucleotide sequence length of 100 nucleotides are compared, no more than 10% (i.e., 10 out of 100) of the nucleotides in the test oligonucleotide differ from those of the reference oligonucleotide. Differences are defined as nucleic acid substitutions, insertions, or deletions.

The term “genetically engineered” as used herein refers to cells that have been manipulated using biotechnology to change the genetic makeup of the cells, including the transfer of genes within and across species boundaries to produce improved or non-naturally occurring cells. A human pluripotent stem cell, macrophage, or neutrophil that contains an exogenous, recombinant, synthetic, and/or otherwise modified polynucleotide is considered to be a genetically engineered cell and, thus, non-naturally occurring relative to any naturally occurring counterpart. In some cases, genetically engineered cells contain one or more recombinant nucleic acids. In other cases, genetically engineered cells contain one or more synthetic or genetically engineered nucleic acids (e.g., a nucleic acid containing at least one artificially created insertion, deletion, inversion, or substitution relative to the sequence found in its naturally occurring counterpart). Procedures for producing genetically engineered cells are generally known in the art, for example, as described in Sambrook et al., Molecular Cloning, A Laboratory Manual (Fourth Edition), Cold Spring Harbor Press, Cold Spring Harbor, N.Y. (2012) and Doudna et al., CRISPR-Cas, A Laboratory Manual, Cold Spring Harbor Press, Cold Spring Harbor, N.Y. (2016).

A genetically engineered cell can be a cell that has been modified using a gene editing technique. Gene editing refers to a type of genetic engineering in which DNA is inserted, deleted, modified, or replaced in the genome of a living cell. In contrast to other genetic engineering techniques that can randomly insert genetic material into a host genome, gene editing can target the insertions to site specific locations (e.g., AAVS1 alleles). Examples of gene editing techniques including, but are not limited to, restriction enzymes, zinc finger nucleases, TALENs, and CRISPR-Cas9.

A genetically engineered cell can be a stem cell (e.g., a human pluripotent stem cell) or any of their differentiated progeny cells (e.g., mesoderm cells, hemangioblast cells, hemogenic endothelium cells, hematopoietic progenitor cells, macrophages, or neutrophils) that have been modified to express, for example, an anti-GD2 CAR. Any of the cells described herein can be genetically engineered. In some embodiments, a genetically engineered cell refers to a cell that is differentiated from a cell that has been genetically engineered (e.g., a macrophage differentiated from a pluripotent stem cell that has undergone gene editing to express anti-GD2 CAR).

The term “tumor cell” as used herein refers to abnormal cells that divide continuously. In some embodiments, the tumor cell is a solid tumor cell. A solid tumor is an abnormal mass of cells that typically does not contain cysts or a liquid area. Examples of solid tumors include, but are not limited to, sarcomas and carcinomas. Cancers of the blood (e.g., leukemias) typically do not form solid tumors. In some embodiments, the “tumor cell” is not a blood cancer cell. “Tumor cells” as used herein refers to a group of tumor cells and/or a single tumor cell.

In some embodiments, the tumor cell expresses GD2. In some embodiments, the tumor cell is a neuroblastoma tumor cell, a melanoma tumor cell, a glioma tumor cell, a soft tissue sarcoma tumor cells, lung cancer cells, a pancreatic cancer, or a breast cancer cell.

### GD2-CAR Macrophages

This disclosure provides anti-cancer macrophages that are genetically engineered (or differentiated from a genetically engineered progenitor cell) to express an anti-disialoganglioside GD2 (GD2) chimeric antigen receptor (CAR).

As used herein, the term “macrophage” (abbreviated as Mφ) refers to a type of white blood cell of a subject's immune system that engulfs and digests (via the process of phagocytosis) anything that does not have, on its surface, proteins that are specific to healthy body cells (including, but not limited to, cancer cells, microbes, viruses, and foreign substances). Macrophages that encourage inflammation and anti-cancer activity are referred to as M1 macrophages and those that decrease inflammation and encourage tissue repair are referred to as M2 macrophages. Human macrophages can be about 20-40 micrometers in diameter and are produced by the differentiation of monocytes. Macrophages can be identified using flow cytometry or immunohistochemical staining based on their expression of proteins including CD14, CD40, CD11b, CD64, F4/80 (mice)/EMR1 (human), lysozyme M, MAC-1/MAC-3 and CD68.

In some embodiments, the genetically engineered macrophages of this disclosure express CD11b and CD14. These cells are referred to as CD11b+ CD14+ macrophages. In other embodiments, the genetically engineered macrophages express higher levels of CD80 and lower levels of CD163 and CD206 than a wild-type macrophage (or a macrophage that has not been genetically engineered).

Any appropriate method can be used to detect expression of biological markers characteristic of cell types described herein. For example, the presence or absence of one or more biological markers can be detected using, for example, RNA sequencing (e.g., RNA-seq), immunohistochemistry, polymerase chain reaction, quantitative real time PCR (qRT-PCR), or other technique that detects or measures gene expression. RNA-seq is a high-throughput sequencing technology that provides a genome-wide assessment of the RNA content of an organism, tissue, or cell. Alternatively, or additionally, one can detect the presence or absence of, or measure the level of, one or more biological markers of HPCs using, for example, Fluorescence in situ Hybridization (FISH; see WO98/45479 published October 1998), Southern blotting, Northern blotting, or polymerase chain reaction (PCR) techniques, such as qRT-PCR. In exemplary embodiments, a cell population obtained according to a method provided herein is evaluated for expression (or the absence thereof) of biological markers of HPCs such as CD34, CD45, CD43, CD49f, and CD90. Quantitative methods for evaluating expression of markers at the protein level in cell populations are also known in the art.

In some embodiments, the genetically engineered macrophage is a CD11b+ CD14+ macrophage that expresses an anti-disialoganglioside GD2 (GD2) chimeric antigen receptor (CAR). The genetically engineered CD11b+ CD14+ macrophages of this disclosure can express any of the anti-GD2 CAR constructs as described herein (e.g., CARa, CAR1, CARb). In some embodiments, the genetically engineered CD11b+ CD14+ macrophage expresses an anti-GD2 CAR comprising a nucleic acid sequence as set forth in any of SEQ ID NOS:1-3 or a sequence having at least 80%, 85%, 90%, 95%, or 99% sequence identity to the nucleic acid sequence as set forth in any of SEQ ID NOS:1-3. In specific embodiments, the genetically engineered CD11b+ CD14+ macrophage expresses an anti-GD2 CAR comprising a nucleic acid sequence as set forth in any of SEQ ID NO:2 or a sequence having at least 80%, 85%, 90%, 95%, or 99% sequence identity to the nucleic acid sequence as set forth in any of SEQ ID NO:2.

In some embodiments, the genetically engineered CD11b+ CD14+ macrophage expresses an anti-GD2 CAR comprising an amino acid sequence as set forth in any of SEQ ID NOS:5-7 or a sequence having at least 80%, 85%, 90%, 95%, or 99% sequence identity to the amino acid sequence as set forth in any of SEQ ID NOS:5-7. In other embodiments, the genetically engineered CD11b+ CD14+ macrophage expresses an anti-GD2 CAR comprising an amino acid sequence as set forth in any of SEQ ID NO:5 or a sequence having at least 80%, 85%, 90%, 95%, or 99% sequence identity to the amino acid sequence as set forth in any of SEQ ID NO:5.

The genetically engineered CD11b+ CD14+ macrophage of the disclosure can express higher levels of CD80 and lower levels of CD163 and CD206 than a wild-type macrophage or a macrophage that does not express the anti-GD2 CAR.

In some embodiments, the genetically engineered CD11b+ CD14+ macrophages of this disclosure exhibit an M1 anti-cancer phenotype. Cells with an M1 anti-cancer phenotype have a pro-inflammatory phenotype with tumor cell killing abilities.

In some embodiments, the macrophage is obtained from a genetically engineered pluripotent stem cell. The term “pluripotent stem cell” as used herein can be an embryonic stem cell (e.g., H1, H9, or BM9 human embryonic stem cell lines) or an induced pluripotent stem cell.

In some embodiments, the genetically engineered CD11b+ CD14+ macrophage that expresses anti-GD2 CAR is capable of inhibiting the survival of a tumor cell. Wild-type or naturally occurring macrophages are not capable of inhibiting the survival of a tumor cell. In some embodiments, the tumor cell expresses GD2. In some embodiments, the genetically engineered CD11b+ CD14+ macrophage that expresses an anti-GD2 CAR selectively targets cells that express GD2 antigen and not cells that do not express GD2 antigen.

In some embodiments, this disclosure provides an isolated population of the CD11b+ CD14+ macrophages that express an anti-GD2 CAR. In some embodiments, the isolated population comprises at least 75%, 80%, 85%, 90%, 91%, 92%, 93%, 94%, 95%, 96%, 97%, 98%, or 99% CD11b+ CD14+ macrophages that express an anti-GD2 CAR.

### GD2-CAR Natural Killer (NK) Cells

This disclosure provides anti-cancer natural killer (NK) cells that are genetically engineered (or differentiated from a genetically engineered cell as set forth in U.S. patent application no. 2020-0080059, expressly incorporated by reference herein) to express an anti-disialoganglioside GD2 (GD2) chimeric antigen receptor (CAR).

As used herein, the term “natural killer cells” (abbreviated as NK) refers to a type of white blood cell that have high cytolytic and cytokine-producing function to kill tumor cells or virus-infected cells.

The CAR natural killer cells produced by the methods described herein express one or more of the following natural killer markers: CD56, KIR, NKp44, NKp46, NKG3D, or NKG2A. In some embodiments, the genetically engineered natural killer cells of this disclosure express CD56 but not CD3. These cells are referred to as CD3− CD56+ natural killer cells.

Any appropriate method can be used to detect expression of biological markers characteristic of cell types described herein. For example, the presence or absence of one or more biological markers can be detected using, for example, RNA sequencing (e.g., RNA-seq), immunohistochemistry, polymerase chain reaction, quantitative real time PCR (qRT-PCR), or other technique that detects or measures gene expression. RNA-seq is a high-throughput sequencing technology that provides a genome-wide assessment of the RNA content of an organism, tissue, or cell. Alternatively, or additionally, one can detect the presence or absence of, or measure the level of, one or more biological markers of HPCs using, for example, Fluorescence in situ Hybridization (FISH; see WO98/45479 published October 1998), Southern blotting, Northern blotting, or polymerase chain reaction (PCR) techniques, such as qRT-PCR. In exemplary embodiments, a cell population obtained according to a method provided herein is evaluated for expression (or the absence thereof) of biological markers of HPCs such as CD34, CD45, CD43, CD49f, and CD90. Quantitative methods for evaluating expression of markers at the protein level in cell populations are also known in the art.

In some embodiments, the genetically engineered natural killer cell is a CD3-CD56+ natural killer cell that expresses an anti-disialoganglioside GD2 (GD2) chimeric antigen receptor (CAR). The genetically engineered CD3− CD56+ natural killer cells of this disclosure can express any of the anti-GD2 CAR constructs as described herein (e.g., CARa, CAR1, CARb). In some embodiments, the genetically engineered CD3− CD56+ natural killer cell expresses an anti-GD2 CAR comprising a nucleic acid sequence as set forth in any of SEQ ID NOS:1-3 or a sequence having at least 80%, 85%, 90%, 95%, or 99% sequence identity to the nucleic acid sequence as set forth in any of SEQ ID NOS:1-3. In other embodiments, the genetically engineered CD3− CD56+ natural killer cell expresses an anti-GD2 CAR comprising a nucleic acid sequence as set forth in any of SEQ ID NO:2 or a sequence having at least 80%, 85%, 90%, 95%, or 99% sequence identity to the nucleic acid sequence as set forth in any of SEQ ID NO:2.

In some embodiments, the genetically engineered CD3− CD56+ natural killer cell expresses an anti-GD2 CAR comprising an amino acid sequence as set forth in any of SEQ ID NOS:5-7 or a sequence having at least 80%, 85%, 90%, 95%, or 99% sequence identity to the amino acid sequence as set forth in any of SEQ ID NOS:5-7. In other embodiments, the genetically engineered CD3− CD56+ natural killer cell expresses an anti-GD2 CAR comprising an amino acid sequence as set forth in any of SEQ ID NO:5 or a sequence having at least 80%, 85%, 90%, 95%, or 99% sequence identity to the amino acid sequence as set forth in any of SEQ ID NO:5.

In some embodiments, the natural killer cell is obtained from a genetically engineered pluripotent stem cell. The term “pluripotent stem cell” as used herein can be an embryonic stem cell (e.g., H1 or H9 human embryonic stem cell lines) or an induced pluripotent stem cell (BM9 and PNMC-3-1 for examples).

In some embodiments, the genetically engineered CD3− CD56+ natural killer cell that expresses an anti-GD2 CAR is capable of killing solid tumor cells. In some embodiments, the tumor cell expresses GD2. In some embodiments, the genetically engineered CD3− CD56+ natural killer cell that expresses an anti-GD2 CAR selectively targets cells that express GD2 antigen and not cells that do not express GD2 antigen. In some embodiments, the tumor cell targeted by the genetically engineered CD3− CD56+ natural killer cell that expresses an anti-GD2 CAR is not a blood cancer.

In other embodiments, the tumor cell that is targeted by the CD3− CD56+ natural killer cell that expresses an anti-GD2 CAR is a neuroblastoma tumor cell, a melanoma tumor cell, a glioma tumor cell, a soft tissue sarcoma tumor cell, a lung cancer cells, a pancreatic cancer cell, or a breast cancer cell.

In some embodiments, this disclosure provides an isolated population of the CD3− CD56+ natural killer cell that express an anti-GD2 CAR. In some embodiments, the isolated population comprises at least 75%, 80%, 85%, 90%, 91%, 92%, 93%, 94%, 95%, 96%, 97%, 98%, or 99% CD3− CD56+ natural killer cell that express an anti-GD2 CAR.

### GD2-CAR Eosinophils

This disclosure provides anti-cancer eosinophils that are genetically engineered (or differentiated from a genetically engineered cell) to express an anti-disialoganglioside GD2 (GD2) chimeric antigen receptor (CAR).

As used herein, the term “eosinophil” (abbreviated as EOS) refers to a type of white blood cell that contain enzymatic granules that are released during infections, allergic reactions, and asthma Eosinophils can play a role in innate defense and anti-tumor surveillance, acting synergistically with other immune cells. Eosinophils are capable of integrating danger signals and responding quickly and selectively. Although the in vivo relevance of these new features attributable to eosinophils remains to be demonstrated, their recruitment to many tumors and their cytotoxic potential (e.g., production of TNF-α, granzyme, cationic proteins, and IL-18) indicates the potential roles for eosinophils as an effector in the anti-tumor response.

The CAR eosinophils produced by the methods described herein express one or more of the following eosinophil markers: EXP1, CD11b, EMR1, CD244, and IL5RA. In some embodiments, the genetically engineered eosinophils of this disclosure express eosinophil peroxidase (EPX). These cells are referred to as EPX+ eosinophil.

Any appropriate method can be used to detect expression of biological markers characteristic of cell types described herein. For example, the presence or absence of one or more biological markers can be detected using, for example, RNA sequencing (e.g., RNA-seq), immunohistochemistry, polymerase chain reaction, quantitative real time PCR (qRT-PCR), or other technique that detects or measures gene expression. RNA-seq is a high-throughput sequencing technology that provides a genome-wide assessment of the RNA content of an organism, tissue, or cell. Alternatively, or additionally, one can detect the presence or absence of, or measure the level of, one or more biological markers of HPCs using, for example, Fluorescence in situ Hybridization (FISH; see WO98/45479 published October 1998), Southern blotting, Northern blotting, or polymerase chain reaction (PCR) techniques, such as qRT-PCR. In exemplary embodiments, a cell population obtained according to a method provided herein is evaluated for expression (or the absence thereof) of biological markers of HPCs such as CD34, CD45, CD43, CD49f, and CD90. Quantitative methods for evaluating expression of markers at the protein level in cell populations are also known in the art.

In some embodiments, the genetically engineered eosinophil is an EPX+ eosinophil that expresses an anti-disialoganglioside GD2 (GD2) chimeric antigen receptor (CAR). The genetically engineered EPX+ eosinophils of this disclosure can express any of the anti-GD2 CAR constructs as described herein (e.g., CARa, CAR1, CARb). In some embodiments, the genetically engineered EPX+ eosinophil expresses an anti-GD2 CAR comprising a nucleic acid sequence as set forth in any of SEQ ID NOS:1-3 or a sequence having at least 80%, 85%, 90%, 95%, or 99% sequence identity to the nucleic acid sequence as set forth in any of SEQ ID NOS:1-3. In other embodiments, the genetically engineered EPX+ eosinophil expresses an anti-GD2 CAR comprising a nucleic acid sequence as set forth in any of SEQ ID NO:2 or a sequence having at least 80%, 85%, 90%, 95%, or 99% sequence identity to the nucleic acid sequence as set forth in any of SEQ ID NO:2.

In some embodiments, the genetically engineered EPX+ eosinophil expresses an anti-GD2 CAR comprising an amino acid sequence as set forth in any of SEQ ID NOS:5-7 or a sequence having at least 80%, 85%, 90%, 95%, or 99% sequence identity to the amino acid sequence as set forth in any of SEQ ID NOS:5-7. In other embodiments, the genetically engineered EPX+ eosinophil expresses an anti-GD2 CAR comprising an amino acid sequence as set forth in any of SEQ ID NO:5 or a sequence having at least 80%, 85%, 90%, 95%, or 99% sequence identity to the amino acid sequence as set forth in any of SEQ ID NO:5.

In some embodiments, the eosinophil is obtained from a genetically engineered pluripotent stem cell. The term “pluripotent stem cell” as used herein can be an embryonic stem cell (e.g., H1, H9, or BM9 human embryonic stem cell lines) or an induced pluripotent stem cell.

In some embodiments, the genetically engineered EPX+ eosinophil that expresses an anti-GD2 CAR is capable of killing solid tumor cells. In some embodiments, the tumor cell expresses GD2. In some embodiments, the genetically engineered EPX+ eosinophil that expresses an anti-GD2 CAR selectively targets cells that express GD2 antigen and not cells that do not express GD2 antigen.

In other embodiments, the tumor cell that is targeted by the EPX+ eosinophil that expresses an anti-GD2 CAR is a neuroblastoma tumor cell, a melanoma tumor cell, a glioma tumor cell, a soft tissue sarcoma tumor cell, a lung cancer cells, a pancreatic cancer cell, or a breast cancer cell.

In some embodiments, this disclosure provides an isolated population of the EPX+ eosinophil that express an anti-GD2 CAR. In some embodiments, the isolated population comprises at least 30%, 35%, 40%, 45%, 49% EPX+ eosinophils that express anti-GD2 CAR. In some embodiments, the isolated population can be purified further to about 75%, 80%, 85%, 90%, 91%, 92%, 93%, 94%, 95%, 96%, 97%, 98%, or 99% EPX+ eosinophil that express an anti-GD2 CAR.

### GD2-CAR Neutrophils

Another aspect of this disclosure provides anti-cancer neutrophils that are genetically engineered to express an anti-disialoganglioside GD2 (GD2) chimeric antigen receptor (CAR).

As used herein, the term “neutrophil” (abbreviated as N) refers to a type of white blood cell that helps the body deal with tissue damage and infections. Neutrophils are the most abundant white blood cell in the human body.

The CAR neutrophils produced by the methods described herein have characteristics of naturally found neutrophils (morphology, phagocytotic behavior, ROS activity, etc.), but are CD10 low/negative (CD10−), which is not a characteristic of naturally found neutrophils. The neutrophils produced in vitro have characteristics different from primary derived neutrophils, for example, the in vitro derived neutrophils are believed to be less efficient at performing NETosis (i.e. form neutrophil extra-cellular traps (NETs) less efficiently compared to peripheral blood neutrophils). This reduction in the ability to perform NETosis and to form NETs can be advantageous when the in vitro derived neutrophils are used for therapeutic purposes, such as treatment of infections or cancer.

The CAR neutrophils produced by the methods described herein express one or more of the following neutrophil marker CD11b, CD16, CD15, MPO, CD182, CD66b, CD95, CD54, and lactoferrin and do not express CD10, signifying a unique in vitro derived population of neutrophils (i.e., CD15+CD10− neutrophils). In some embodiments, the genetically engineered neutrophil expresses CD11b and CD15. These cells are referred to as CD11b+ CD15+ neutrophil. In some embodiments, the genetically engineered neutrophil does not express or express low levels of CD10.

CAR neutrophils can be detected by histological staining and flow cytometry using forward and side scatter, as neutrophils have a distinct phenotype and can readily be distinguished from other blood cells (macrophages and lymphocytes) by flow cytometry just by size as readily understood by one skilled in the art. Further, neutrophils have a distinct morphology, as they are non-adherent cells which has a nucleus divided into 2-5 lobes which can be readily detected by histological staining. The population of CAR neutrophils produced by the methods described herein also have phagocytic, chemotactic and signaling functions of primary human neutrophils.

Accordingly, in some embodiments, the genetically engineered neutrophil is a CD11b+ CD15+ neutrophil that expresses an anti-disialoganglioside GD2 (GD2) chimeric antigen receptor (CAR). The genetically engineered CD11b+ CD15+ neutrophils of this disclosure can express any of the anti-GD2 CAR constructs as described herein (e.g., CARa, CAR1, CARb). In some embodiments, the genetically engineered CD11b+ CD15+ neutrophil expresses an anti-GD2 CAR comprising a nucleic acid sequence as set forth in any of SEQ ID NOS:1-3 or a sequence having at least 80%, 85%, 90%, 95%, or 99% sequence identity to the nucleic acid sequence as set forth in any of SEQ ID NOS:1-3. In other embodiments, the genetically engineered CD11b+ CD15+ neutrophil expresses an anti-GD2 CAR comprising a nucleic acid sequence as set forth in any of SEQ ID NO:2 or a sequence having at least 80%, 85%, 90%, 95%, or 99% sequence identity to the nucleic acid sequence as set forth in any of SEQ ID NO:2.

In some embodiments, the genetically engineered neutrophil is a CD11b+ CD15+ neutrophil that expresses an anti-GD2 CAR comprising an amino acid sequence as set forth in any of SEQ ID NOS:5-7 or a sequence having at least 80%, 85%, 90%, 95%, or 99% sequence identity to the amino acid sequence as set forth in any of SEQ ID NOS:5-7. In other embodiments, the genetically engineered CD11b+ CD15+ neutrophil expresses an anti-GD2 CAR comprising an amino acid sequence as set forth in any of SEQ ID NO:5 or a sequence having at least 80%, 85%, 90%, 95%, or 99% sequence identity to the amino acid sequence as set forth in any of SEQ ID NO:5.

In some embodiments, the genetically engineered CD11b+ CD15+ neutrophil that expresses an anti-GD2 CAR is produced from a genetically engineered pluripotent stem cell. In some embodiments, the pluripotent stem cell is an embryonic stem cell or an induced pluripotent stem cell.

In some embodiments, the genetically engineered CD11b+ CD15+ neutrophil that expresses an anti-GD2 CAR is capable of killing solid tumor cells. In some embodiments, the tumor cell expresses GD2. In some embodiments, the genetically engineered CD11b+ CD15+ neutrophil that expresses an anti-GD2 CAR selectively targets cells that express GD2 antigen and not cells that do not express GD2 antigen. In some embodiments, the tumor cell targeted by the genetically engineered CD11b+ CD15+ neutrophil that expresses an anti-GD2 CAR is not a blood cancer.

In other embodiments, the tumor cell that is targeted by the CD11b+ CD15+ neutrophil that expresses an anti-GD2 CAR is a neuroblastoma tumor cell, a melanoma tumor cell, a glioma tumor cell, a soft tissue sarcoma tumor cell, a lung cancer cells, a pancreatic cancer cell, or a breast cancer cell.

In some embodiments, this disclosure provides an isolated population of the CD11b+ CD15+ neutrophils that express an anti-GD2 CAR. In some embodiments, the isolated population comprises at least 75%, 80%, 85%, 90%, 91%, 92%, 93%, 94%, 95%, 96%, 97%, 98%, or 99% CD11b+ CD15+ neutrophils that express an anti-GD2 CAR.

### Methods for Producing GD2-CAR Immune Cells

This disclosure also provides methods for producing cells that express anti-GD2 CAR. In particular, methods for producing a pluripotent stem cell expressing an anti-GD2 CAR, a CD11b+ CD14+ macrophage expressing an anti-GD2 CAR, a CD11b+ CD15+ neutrophil expressing an anti-GD2 CAR, a CD3− CD56+ natural killer cell expressing an anti-GD2 CAR, and an EPX+ eosinophil expressing an anti-GD2 CAR are provided herein. In some embodiments, a nucleic acid vector encoding the chimeric antigen receptor is transfected in human pluripotent stem cells, mesoderm cells, hemangioblasts, hemogenic endothelium cells or hematopoietic progenitor cells for use in any of the methods described herein to produce CAR macrophages, CAR neutrophils, CAR natural killer cell, or CAR eosinophil. In other embodiments, a nucleic acid vector encoding the anti-GD2 chimeric antigen receptor is transfected in human pluripotent stem cells and then the human pluripotent stem cells can be differentiated to produce a progeny cells (e.g., mesoderm cells, hemangioblast cells, hemogenic endothelium cells, hematopoietic progenitor cells, macrophages, endothelial progenitor cells, myeloid progenitor cells, or neutrophils) that also express an anti-GD2 CAR. The neutrophils of this disclosure can be produced from human induced pluripotent stem cells (hiPSCs) via direct hematoendothelial programming using ETV2 modified mRNA, as described in U.S. Patent Application Publication No. 2020-0385676, incorporated herein by reference in its entirety.

The production of hematopoietic progenitor/stem cells and macrophages can be accomplished by the methods disclosed in U.S. Patent Application Publication No. 2020-0080059, incorporated herein by reference in its entirety. In some embodiments, the pluripotent stem cell is an embryonic stem cell or an induced pluripotent stem cell.

Human pluripotent stem cells (hPSCs), either embryonic or induced, provide access to the earliest stages of human development and offer a platform on which to derive a large number of hematopoietic progenitor cells or blood cells for cellular therapy and tissue engineering. Accordingly, the methods provided herein can comprise differentiating human pluripotent stem cells under conditions that promote differentiation of mesodermal cells (e.g., arterial endothelial cells) into hematopoietic progenitor cells into macrophages, neutrophils, natural killer cells, and/or eosinophils.

In exemplary embodiments, pluripotent stem cells are cultured at low density in a chemically defined culture medium comprising or consisting essentially of DMEM/F12 culture medium, L-ascorbic acid-2-phosphate magnesium, sodium selenium, human FGF2, insulin, NaHCO₃, transferrin, TGFβ1, BMP4, Activin-A, and CHIR99021 (“E8BAC medium”) for two days. The culture medium can comprise or consist essentially of DMEM/F12 medium; L-ascorbic acid-2-phosphate magnesium (64 mg/L); sodium selenium (14 μg/L); human FGF2 (100 μg/L); insulin (20 mg/L); NaHCO3 (543 mg/L); transferrin (10.7 mg/L); TGFβ1 (2 μg/L); BMP4 (5 ng/mL); Activin A (25 μg/L); and CHIR99021 (1 μM). Human pluripotent stem cells can be cultured in the culture medium for about two days. After about two days, at least about 80% (e.g., at least about 80%, 85%, 90%, 95%, or 99%) of the resulting cell population are mesoderm cells.

As used herein, the term “mesoderm cell” refers to a cell having mesoderm-specific gene expression, capable of differentiating into a mesodermal lineage such as bone, muscle such as cardiac muscle, skeletal muscle, and smooth muscle (e.g., of the gut), connective tissue such as the dermis and cartilage, kidneys, the urogenital system, blood or hematopoietic cells, heart, and vasculature. Mesoderm-specific biomarkers include Brachyury (7). Culturing can take place on any appropriate surface (e.g., in two-dimensional or three-dimensional culture).

Medium and substrate conditions for culturing pluripotent stem cells, as used in the methods described herein, are well known in the art. In some cases, pluripotent stem cells to be differentiated according to the methods disclosed herein are cultured in mTESR-1@ medium (StemCell Technologies, Inc., Vancouver, British Columbia.), E8 medium, or Essential 8® medium (Life Technologies, Inc.) on a MATRIGEL™ substrate (BD Biosciences, NJ) according to the manufacturer's protocol or on a Corning™ Synthemax™ surface.

Exemplary media that can be used to culture the cells of this disclosure are provided in Table 1 and Table 2.

TABLE 1
Chemically defined Culture Medium Components

| Medium Name | Chemically defined Components |
|---|---|
| E8 | DMEM/F12 medium + |
| | L-ascorbic acid-2-phosphate magnesium (64 mg/l); |
| | sodium selenium (14 μg/l); |
| | human FGF2(100 μg/l); |
| | insulin (20 mg/l); |
| | NaHCO₃ (543 mg/l); |
| | Transferrin (10.7 mg/l); and |
| | TGFβ1 (2 μg/l) |
| E8BAC | E8 medium + |
| | BMP4 (5 ng/mL); |
| | Activin A (25 μg/l); and |
| | CHIR99021 (1 μM) |
| E5 | DMEM/F12 medium + |
| | L-ascorbic acid-2-phosphate magnesium (64 mg/l); |
| | sodium selenium (14 μg/l); |
| | NaHCO₃ (543 mg/l); and |
| | transferrin (10.7 mg/l) |
| FVIRL | E5 medium + |
| “Five Factor” | Human FGF2 (100 μg/l) |
| | VEGF-165 (50 μg/l) |
| | SB431542 (10 μM) |
| | RESV (5 μM) |
| | L-690,330 (10 μM) |
| E6 | DMEM/F12 medium + |
| | L-ascorbic acid-2-phosphate magnesium (64 mg/l); |
| | sodium selenium (14 μg/l); |
| | insulin (20 mg/l); |
| | NaHCO₃ (543 mg/l); and |
| | transferrin (10.7 mg/l) |
| FVR | E6 medium + |
| | Human FGF2 (100 μg/l); |
| | VEGF-165 (50 μg/l); |
| | Resveratrol (5 μM) |
| E6G | E6 medium + |
| | GM-CSF (200 ng/mL) |
| E6M | E6 medium + |
| | IL-1B (10 ng/mL) |
| | M-CSF (20 ng/mL) |
| E6T | E6 medium + |
| | TGFβ1 (2 ng/mL) |
| StemLine II | StemLine II + |
| | 20 ng/mL of human FGF2 |
| StemLine II | StemLine II + |
| | 20 ng/mL of human FGF2 |
| | GM-CSF (25 ng/mL) |
| | UM171 (50 nM) |
| StemSpanH300 | StemSpanH300 medium + |
| medium | GlutaMAX 100X |
| | ExCyte 0.2% |
| | human G-CSF |
| | Am580 retinoic acid agonist 2.5 μM |
| | gentamycin (1,000×) |

TABLE 2

| Medium | S-B | NKM | EM | |
|---|---|---|---|---|
| components | medium | medium | medium | M36 |
| Base | E6 | E6 | E6 | E6 |
| IL-7 | — | 20 ng/ml | — | |
| IL-15 | — | 10 ng/ml | — | |
| SCF | 20-50 ng/ml | 20 ng/ml | — | |
| FLT3-L | — | 10 ng/ml | — | |
| IL-3 | | | 10 ng/ml | 10 ng/ml |
| IL-5 | | | 10 ng/ml | |
| IL6 | | | | 20 ng/ml |
| M-CSF | | | | 20 ng/ml |
| Serum | 20% BIT9500 | 10% KOSR | 10% KOSR | 10% KOSR |
| replacements | | | | |

For example, E8BAC medium (Table 1, Zhang et al., 2017, PNAS 114(30): E6072-E6078) can be used to differentiate human pluripotent stem cells to mesodermal cells. The “Five Factor” medium can be used to differentiate pluripotent stem cell-derived mesodermal cells into hemangioblasts and hemogenic endothelium cells. The FVR medium can be used to differentiate hemangioblasts and arterialized hemogenic endothelium cells into hematopoietic progenitor cells. The E6G (or M36 media, E6 +20 ng/ml M-CSF +10 ng/ml IL3, +20 ng/ml TL6), and E6M media can be used to differentiate hematopoietic progenitor cells into macrophages. The StemLine II and StemSpanH300 media (supplemented as shown in Table 1) can be used to produce neutrophils from hPSCs. The S-B and NKM medium can be used to differentiate hematopoietic progenitor cells into natural killer cells. The EM medium can be used to differentiate hematopoietic progenitor cells into eosinophils.

Human pluripotent stem cells (e.g., human ESCs or iPS cells) can be cultured in the absence of a feeder layer (e.g., a fibroblast feeder layer), a conditioned medium, or a culture medium comprising poorly defined or undefined components.

As used herein, the term “albumin-free conditions” indicates that the culture medium used contains no added albumin in any form including, without limitation, Bovine Serum Albumin (BSA), any form of recombinant albumin, or any other animal albumin.

As used herein, the terms “chemically defined medium” and “chemically defined culture medium” also refer to a culture medium containing formulations of fully disclosed or identifiable ingredients, the precise quantities of which are known or identifiable and can be controlled individually. As such, a culture medium is not chemically defined if (1) the chemical and structural identity of all medium ingredients is not known, (2) the medium contains unknown quantities of any ingredients, or (3) both. Standardizing culture conditions by using a chemically defined culture medium minimizes the potential for lot-to-lot or batch-to-batch variations in materials to which the cells are exposed during cell culture. Accordingly, the effects of various differentiation factors are more predictable when added to cells and tissues cultured under chemically defined conditions.

As used herein, the term “serum-free” refers to cell culture materials that do not contain serum or serum replacement, or that contains essentially no serum or serum replacement. For example, an essentially serum-free medium can contain less than about 1%, 0.9%, 0.8%, 0.7%, 0.6%, 0.5%, 0.4%, 0.3%, 0.2%, or 0.1% serum. “Serum free” also refers to culture components free of serum obtained from animal (e.g., fetal bovine) blood or animal-derived materials, which is important to reduce or eliminate the potential for cross-species viral or prion transmission. For avoidance of doubt, serum-containing medium is not chemically defined.

As used herein, “feeder-free” refers to culture conditions that are substantially free of a cell feeder layer. Cells grown under feeder-free conditions can be grown on a substrate, such as a chemically defined substrate, and/or grown as an adherent culture. Suitable chemically defined substrates include vitronectin.

As used herein, the terms “seeded at low density” or “cultured at low density” refer to a cell culture seeded at a density of between about 6×10³ cells/cm² and about 6×10⁴ cells/cm² (e.g., about 6×10³ cells/cm², 6.5×10³ cells/cm², 7×10³ cells/cm², 7.5×10³ cells/cm², 8×10³ cells/cm², 8.5×10³ cells/cm², 9×10³ cells/cm², 1×10⁴ cells/cm², 1.5×10⁴ cells/cm², 2×10⁴ cells/cm², 2.5×10⁴ cells/cm², 3×10⁴ cells/cm², 3.5×10⁴ cells/cm², 4×10⁴ cells/cm², 4.5×10⁴ cells/cm², 5×10⁴ cells/cm². 5.5×10⁴ cells/cm², 6×10⁴ cells/cm²). In some embodiments, seeded at a low density can be about 1.8×10⁴/cm².

As used herein, the terms “seeded at high density” or “cultured at high density” refer to a cell culture seeded at a density of above about 6×10⁴ cells/cm² and up to about 3×10⁵ cells/cm² (e.g., about 6×10⁴ cells/cm², 6.5×10⁴ cells/cm², 7×10⁴ cells/cm², 7.5×10⁴ cells/cm², 8×10⁴ cells/cm², 8.5×10⁴ cells/cm², 9×10⁴ cells/cm², 1×10⁵ cells/cm², 1.5×10⁵ cells/cm², 2×10⁵ cells/cm², 2.5×10⁵ cells/cm², 3×10⁵ cells/cm²). In some embodiments, seeded at high density can be about 1.1×10⁵ cells/cm².

A method of producing a hematopoietic progenitor cell can comprise culturing human pluripotent stem cells in a serum-free, albumin-free, chemically defined culture medium that promotes differentiation to mesoderm. In this manner, pluripotent stem cell-derived mesodermal cells are differentiated according to the HPC differentiation methods provided herein, thus producing pluripotent stem cell-derived HPCs.

As used herein, “pluripotent stem cells” appropriate for use according to a method of the invention are cells having the capacity to differentiate into cells of all three germ layers. Suitable pluripotent cells for use herein include human embryonic stem cells (hESCs) and human induced pluripotent stem (iPS) cells. As used herein, “embryonic stem cells” or “ESCs” mean a pluripotent cell or population of pluripotent cells derived from an inner cell mass of a blastocyst. See Thomson et al., 1998, Science 282:1145-1147. These cells can express Oct-4, SSEA-3, SSEA-4, TRA-1-60, and TRA-1-81. Pluripotent stem cells appear as compact colonies comprising cells having a high nucleus to cytoplasm ratio and prominent nucleolus. ESCs are commercially available from sources such as WiCell Research Institute (Madison, WI.).

As used herein, “induced pluripotent stem cells” or “iPS cells” or “iPSCs” refers to pluripotent cell or population of pluripotent cells that can vary with respect to their differentiated somatic cell of origin, that can vary with respect to a specific set of potency-determining factors and that can vary with respect to culture conditions used to isolate them, but nonetheless are substantially genetically identical to their respective differentiated somatic cell of origin and display characteristics similar to higher potency cells, such as ESCs, as described herein. See, e.g., Yu et al., Science 318:1917-1920 (2007).

Induced pluripotent stem cells exhibit morphological properties (e.g., round shape, large nucleoli, and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60, or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained from a post-natal individual.

The methods provided herein produce isolated populations of pluripotent stem cell-derived CAR macrophages, CAR natural killer cells, CAR eosinophils and CAR neutrophils, where the isolated population is a substantially pure population of CAR macrophages, CAR natural killer cells, CAR eosinophils and CAR neutrophils. As used herein, “isolating” and “isolated” refer to separating, selecting, or enriching for a cell type of interest or subpopulation of cells from surrounding, neighboring, or contaminating cells or from cells of another type. As used herein, the term “substantially pure” refers to a population of cells that is at least about 80% (e.g., at least about 80%, 82%, 83%, 85%, 90%, 95%, 96%, 97%, 98%, 99% or more) pure, with respect CAR macrophages, CAR natural killer cells, CAR eosinophils and CAR neutrophils making up a total cell population. In other words, the term “substantially pure” refers to a population of CAR macrophages, CAR natural killer cells, CAR eosinophils and CAR neutrophils of this disclosure that contains at least about 80% (e.g., at least about 80%, 82%, 83%, 85%, 90%, 95%, 96%, 97%, 98%, 99% or more) of CAR macrophages, CAR natural killer cells, CAR eosinophils and CAR neutrophils respectively when directing differentiation to obtain cells of the hematopoietic progenitor cell lineage.

The term “substantially pure” also refers to a population CAR macrophages, CAR natural killer cells, CAR eosinophils and CAR neutrophils of this invention that contains fewer than about 20%, about 10%, or about 5% of non-CAR macrophages, non-CAR natural killer cells, non-CAR eosinophils, and/or non-CAR neutrophils, in a population prior to any enrichment, expansion step, separation, or selection step. In some cases, a substantially pure isolated population of CAR macrophages, CAR neutrophils, and CAR natural killer cells generated according to a method provided herein is at least about 95% (e.g., at least about 95%, 96%, 97%, 98%, 99%) pure with respect to CAR macrophages, CAR neutrophils, and CAR natural killer cells making up a total cell population. In some cases, an isolated population of CAR eosinophils generated according to a method provided herein is at least about 30% pure with respect to CAR eosinophils making up a total cell population. A substantially pure isolated population of CAR eosinophils of at least about 95% (e.g., at least about 95%, 96%, 97%, 98%, 99%) pure can be achieved by further purification methods commonly known in the arts.

In some embodiments, the proportion of CAR macrophages, CAR natural killer cells, CAR eosinophils and CAR neutrophils in a population of cells obtained in the described methods can be enriched using a cell separation, cell sorting, or enrichment method, e.g., fluorescence activated cell sorting (FACS), enzyme-linked immunosorbent assay (ELISA), magnetic beads, magnetic activated cell sorting (MACS), laser-targeted ablation of non-endothelial cells, and combinations thereof. Preferably, FACS is used to identify and separate cells based on cell-surface antigen expression.

The methods of this disclosure provide scalable, inexpensive, and reproducible generation of CAR macrophages, CAR natural killer cells, CAR eosinophils and CAR neutrophils. For instance, after obtaining a cell population comprising CAR macrophages, CAR natural killer cells, CAR eosinophils and CAR neutrophils according to a method described herein, the CAR macrophages, CAR natural killer cells, CAR eosinophils and CAR neutrophils population can be expanded in a culture medium appropriate for proliferating human CAR macrophages, CAR natural killer cells, CAR eosinophils and CAR neutrophils, without limitation, the E6M medium (for CAR macrophages) and StemSpan H3000 medium.

In some embodiments, a method for producing a CD11b+ CD14+ macrophage that expresses an anti-GD2 CAR can comprise: (a) genetically engineering a pluripotent stem cell to express an anti-GD2 chimeric antigen receptor (CAR); and (b) culturing the pluripotent stem cell in a first chemically defined medium for a sufficient time to produce a mesoderm cell (e.g., E8BAC medium); (c) culturing the mesoderm cell seeded at low density in a second chemically defined culture medium that comprises a fibroblast growth factor (FGF) and a vascular endothelial growth factor (VEGF) (e.g., Five Factor medium) for a sufficient time to produce a hemangioblast cell; (d) culturing the hemogenic endothelial cells in a third chemically defined culture medium (e.g., FVR medium or E6 medium including stem cell factor (SCF), IL-3, and thrombopoietin (TPO)) for a sufficient time to produce a CD34+ CD45+ hematopoietic progenitor cells; (e) culturing the CD34+ CD45+ hematopoietic progenitor cell in a fourth and/or fifth chemically defined culture medium (e.g., E6G and/or E6M media) for a sufficient time to produce a CD11b+ CD14+ macrophage.

In other embodiments, a method for producing a CD11b+ CD14+ macrophage that expresses an anti-GD2 CAR, the method can comprise: (a) genetically engineering a hematopoietic progenitor cell (HPC) to express an anti-GD2 chimeric antigen receptor (CAR), wherein the HPC was produced through arterial endothelial to hematopoietic transition; and (b) culturing the hematopoietic progenitor cell in a feeder-free and serum-free medium for a sufficient time to produce the CD11b+ CD14+ macrophage.

In an exemplary embodiment, the floating cells of the cell population comprising hematopoietic progenitor cells are cultured in in E6G medium for about 3 days and then E6M medium (supplemented with 10% FBS or KOSR) for about another 6 days. The protocol can generate more than 95% of CD11b+ CD14+ macrophages that are functional (e.g., able to perform phagocytosis).

In some embodiments, the population of CAR macrophages produced by the methods described herein can comprises at least about 90% (or at least about 95%) CD11b⁺ CD14⁺ macrophages.

In some embodiments, a method for producing a CD3− CD56+ natural killer cell that expresses an anti-GD2 CAR can comprise: (a) genetically engineering a pluripotent stem cell to express an anti-GD2 chimeric antigen receptor (CAR); and (b) culturing the pluripotent stem cell in a first chemically defined medium for a sufficient time to produce a mesoderm cell (e.g., E8BAC medium); (c) culturing the mesoderm cell seeded at low density in a second chemically defined culture medium that comprises a fibroblast growth factor (FGF) and a vascular endothelial growth factor (VEGF) (e.g., Five Factor medium) for a sufficient time to produce a hemangioblast cell; (d) culturing the hemogenic endothelial cells in a third chemically defined culture medium (e.g., FVR medium or E6 medium including stem cell factor (SCF), IL-3, and thrombopoietin (TPO)) for a sufficient time to produce a CD34+ CD45+ hematopoietic progenitor cells; (e) culturing the CD34+ CD45+ hematopoietic progenitor cell in a fourth and/or fifth chemically defined culture medium (e.g., S-B and/or NKM media) for a sufficient time to produce a CD3− CD56+ natural killer cell.

In other embodiments, a method for producing a CD3− CD56+ natural killer cell that expresses an anti-GD2 CAR, the method can comprise: (a) genetically engineering a hematopoietic progenitor cell (HPC) to express an anti-GD2 chimeric antigen receptor (CAR), wherein the HPC was produced through arterial endothelial to hematopoietic transition; and (b) culturing the hematopoietic progenitor cell in a feeder-free and serum-free medium for a sufficient time to produce the CD3− CD56+ natural killer cell.

In some embodiments, the population of CAR natural killer cells produced by the methods described herein can comprises at least about 90% (or at least about 95%) CD3− CD56+ natural killer cells.

In some embodiments, a method for producing an EPX+ eosinophils that express an anti-GD2 CAR can comprise: (a) genetically engineering a pluripotent stem cell to express an anti-GD2 chimeric antigen receptor (CAR); and (b) culturing the pluripotent stem cell in a first chemically defined medium for a sufficient time to produce a mesoderm cell (e.g., E8BAC medium); (c) culturing the mesoderm cell seeded at low density in a second chemically defined culture medium that comprises a fibroblast growth factor (FGF) and a vascular endothelial growth factor (VEGF) (e.g., Five Factor medium) for a sufficient time to produce a hemangioblast cell; (d) culturing the hemogenic endothelial cells in a third chemically defined culture medium (e.g., FVR medium or E6 medium including stem cell factor (SCF), IL-3, and thrombopoietin (TPO)) for a sufficient time to produce a CD34+ CD45+ hematopoietic progenitor cells; (e) culturing the CD34+ CD45+ hematopoietic progenitor cell in a fourth and/or fifth chemically defined culture medium (e.g., EM medium) for a sufficient time to produce an EPX+ eosinophil.

In other embodiments, a method for producing an EPX+ eosinophil that expresses an anti-GD2 CAR, the method can comprise: (a) genetically engineering a hematopoietic progenitor cell (HPC) to express an anti-GD2 chimeric antigen receptor (CAR), wherein the HPC was produced through arterial endothelial to hematopoietic transition; and (b) culturing the hematopoietic progenitor cell in a feeder-free and serum-free medium for a sufficient time to produce the EPX+ eosinophil.

In some embodiments, the population of CAR eosinophils produced by the methods described herein can comprises at least about 30% (or at least about 40%) EPX+ eosinophils.

Another aspect of this disclosure provides a method for producing a CD11b+ CD15+ neutrophil that expresses an anti-GD2 CAR, the method comprising: (a) genetically engineering a pluripotent stem cell (PSC) to express an anti-GD2 chimeric antigen receptor (CAR); (b) introducing exogenous ETV2 in the genetically engineered PSC and culturing the ETV2-induced PSC in a xenogen-free, feeder-free, and serum-free medium to produce a population of ETV2-induced endothelial progenitor cells; (c) culturing the ETV2-induced endothelial progenitor cells in xenogen-free, feeder-free, and serum-free medium comprising for a sufficient time to produce non-adherent myeloid progenitors; and (d) culturing the myeloid progenitors in xenogen-free, feeder-free, and serum-free medium for a sufficient time to differentiate the non-adherent myeloid progenitors into CD11b+ CD15+ neutrophils. Steps (b)-(d) of the method to produce a CD11b+ CD15+ neutrophil that expresses an anti-GD2 CAR can be performed according to the methods described in U.S. Patent Application Publication No. 2020-0385676, incorporated herein by reference in its entirety.

Briefly, in step (b), PSCs are directly programmed into hematoendothelial progenitors using ETV2 modified mRNA (mmRNA) which transiently produced ETV2 within the cells. In step (c), the hematoendothelial progenitors are then differentiated into myeloid progenitors in the presence of GM-CSF, FGF2 and optionally UM171 (the presence of UM171 in combination with GM-CSF and FGF2 increases the number of neutrophils produced by the methods). Myeloid progenitors which are non-adherent can be continuously collected from cultures every 8-10 days for up to 30 days of post ETV2 transfection. In step (d), these myeloid progenitors are subsequently differentiated into mature neutrophils in the presence of G-CSF and a retinoic acid agonist (e.g., Am580). This method significantly expedites generation of neutrophils with the first batch of neutrophils available as soon as 14 days after initiation of differentiation and allows the generation of up to 1.7×10⁷ neutrophils from 10⁶ hPSCs. The produced in vitro derived neutrophil cells are suitable for generating mature functional granulocytic cells for the treatment of solid tumors that express, for example, GD2.

In one embodiment, an in vitro method of producing CAR neutrophils from pluripotent stem cells (PSCs) is provided. The method comprises (a) genetically engineering PSCs to express an anti-GD2 CAR; (b) transiently introducing exogenous ETV2 in the PSCs and culturing the ETV2-induced PSCs in xenogen-free medium comprising FGF-2 to produce a population of ETV2-induced CD 144+ hematoendothelial progenitor cells; (c) culturing the ETV2-induced CD144+ hematoendothelial progenitor cells in xenogen-free medium comprising GM-CSF and FGF2 for a sufficient time to produce non-adherent (e.g., floating) myeloid progenitors (e.g., CD34+ CD45+ myeloid progenitors); and (d) culturing the myeloid progenitors in xenogen-free medium comprising G-CSF and retinoic acid agonist to differentiate the myeloid progenitors into CD11b+ CD15+ neutrophils.

A sufficient time for step (b) comprises culturing the ETV2-induced cells for about 2-8 days, for example, for about 4 days. For example, in some embodiments, step (b) comprises culturing for 2 days, alternatively 3 days, alternatively 4 days, alternatively 5 days, alternatively 6 days, alternatively 7 days, alternatively 8 days to produce ETV2-induced CD 144+ hematoendothelial progenitor cells.

The ETV2-induced CD144+ hematoendothelial progenitor cells are characterized by the expression on their surface of CD 144 (CD 144+), and do not express CD73, CD235a or CD43 (CD73−CD235a−CD43−).

Step (c) comprises culturing the ETV2-induced CD144+ hematoendothelial progenitor cells in serum- and xenogen-free medium comprising GM-CSF and FGF2 for a sufficient time to produce myeloid progenitors. In some embodiments, the serum- and xenogen-free medium comprising GM-CSF, FGF-2, and UM 171.

Step (c) comprises culturing the cells for a sufficient time to produce non-adherent myeloid progenitors (e.g., CD34+ CD33+CD45+ myeloid progenitors), for example, at least 3 days, for example, at least 4-23 days.

Step (d) comprises culturing the CD34+ CD45+ myeloid progenitors in xenogen-free medium (e.g., StemSpan™ H3000, StemCell Technologies) comprising G-CSF and a retinoic acid agonist for a sufficient amount of time to differentiate the CD34+ CD45+ myeloid progenitors and produce CD11b+ CD15+ neutrophils. The serum-free, xenogen-free medium suitably comprises a sufficient amount of G-CSF and retinoic acid agonist to produce neutrophils from CD34+ CD45+ myeloid progenitors. Suitable amounts of G-CSF and retinoic acid agonist include, for example, about 100 ng/ml to about 200 ng/ml of G-CSF, and about 1 μm to about 5 μM of the retinoic acid agonist. In another example, the ranges are about 120 ng/ml to about 180 ng/ml of G-CSF, and about 2 μm to about 4 μM retinoic acid agonist.

Suitable retinoic acid agonists, or retinoic acid receptor, alpha (RARa) agonists are known in the art and commercially available, and include, but are not limited to, for example, AM580, Adapalene, AM 80, BMS 753, BMS 961, CD 1530, CD2314, CD 437, Ch55, Isotretinoin, Tazarotene, TTNPB, and retinoic acid, among others.

Step (d) is performed for a sufficient time in order produce neutrophils from CD34+CD33+CD45+ myeloid progenitors by in vitro differentiation. In some examples, step (d) is carried out for at least 7 days, for example, at least 7-21 days. In some embodiments, mature anti-GD2 CAR neutrophils can be harvested after about 8 days of culture.

In some embodiments, transient expression of ETV2 in hPSCs is achieved by any of a number of established methods to introduce a mammalian expression vector, e.g., lipofection, electroporation, or nucleofection, into the cell. In some embodiments, mammalian expression vectors to be used are double-stranded nucleic acid vectors (e.g., episomal plasmid vectors, transposon vectors, or minicircle vectors). Mammalian expression vectors suitable for the methods described herein comprise a promoter competent to drive transient ETV2 expression in hPSCs and encode for the ETV2 protein. Suitable vectors are known in the art.

### Manipulation of SIRPa Expression

SIRPa is abundantly expressed in macrophages, dendritic cells, and neutrophils, and can inhibit anti-tumor activity of these cells. In one embodiment, CAR-M cells used in the methods disclosed herein have inhibited expression of SIRPa. Expression of SIRPa can also be inhibited in CAR-N cells. SIRPa is a ligand for the ubiquitously expressed protective (“don't-eat-me”) signal molecule CD47. SIRPa also promotes M2 polarization of tumor-associated macrophages.

“Having inhibited expression of SIRPa,” indicates that the gene is repressed or not expressed in a functional protein form. In particular embodiments, the expression of SIRPPα is knocked out such that there is no expression of SIRPa. This inhibition or knockout can be obtained by gene mutation, RNA-mediated inhibition, RNA editing, DNA gene editing or base editing.

In particular embodiments, the gene editing method comprises the use of a nuclease selected from a meganuclease, zinc-finger nucleases (ZFNs), transcription activator-like effector nucleases (TALENs), and Cas enzyme. In particular embodiments, the nuclease is a Cas9 enzyme.

### Pharmaceutical Formulations and Methods of Treatment

In exemplary embodiments, CAR macrophages, CAR natural killer cells, CAR eosinophils and CAR neutrophils of this disclosure are provided to the subject as a pharmaceutical composition comprising the cells and one or more pharmaceutically acceptable carriers, buffers, or excipients. The pharmaceutical composition for administration must be formulated, produced, and stored according to standard methods that provide proper sterility and stability.

Preparations comprising CAR macrophages, CAR natural killer cells, CAR eosinophils and CAR neutrophils useful for clinical applications must be obtained in accordance with regulations imposed by governmental agencies such as the U.S. Food and Drug Administration. Accordingly, in exemplary embodiments, the methods provided herein are conducted in accordance with Good Manufacturing Practices (GMPs), Good Tissue Practices (GTPs), and Good Laboratory Practices (GLPs). Reagents comprising animal derived components are not used, and all reagents are purchased from sources that are GMP-compliant. In the context of clinical manufacturing of a cell therapy product, such as in vitro populations of CAR macrophages and CAR neutrophils, GTPs govern donor consent, traceability, and infectious disease screening, whereas the GMP is relevant to the facility, processes, testing, and practices to produce a consistently safe and effective product for human use. See Lu et al., 2009, Stem Cells 27: 2126-2135. Where appropriate, oversight of patient protocols by agencies and institutional panels is envisioned to ensure that informed consent is obtained; safety, bioactivity, appropriate dosage, and efficacy of products are studied in phases; results are statistically significant; and ethical guidelines are followed.

Another aspect of the disclosure provides a method of treating a solid tumor in a subject in need thereof, the method comprising administering a therapeutically effective amount of a genetically engineered CD11b+ CD14+ macrophage that expresses anti-GD2 CAR, genetically engineered CAR natural killer cell, genetically engineered CAR eosinophil, genetically engineered CD11b+ CD15+ neutrophil that expresses anti-GD2 CAR, or combinations thereof.

Yet another aspect of the disclosure provides a method for reducing the proliferation of a solid tumor cell, the method comprising contacting the solid tumor with a genetically engineered CD11b+ CD14+ macrophage that expresses anti-GD2 CAR, genetically engineered CD3− CD56+ natural killer cell, genetically engineered EPX+ eosinophil, genetically engineered CD11b+ CD15+ neutrophil that expresses anti-GD2 CAR, or combinations thereof.

In some embodiments, the methods of this disclosure comprise administering a genetically engineered CD11b+ CD14+ macrophage, genetically engineered CD3− CD56+ natural killer cell, genetically engineered EPX+ eosinophil, or genetically engineered CD11b+ CD15+ neutrophil, that expresses an antigen-specific extracellular domain that recognizes a first tumor antigen and a genetically engineered CD11b+ CD14+ macrophage, genetically engineered CD3− CD56+ natural killer cell, genetically engineered EPX+ eosinophil, or genetically engineered CD11b+ CD15+ neutrophil that expresses an antigen-specific extracellular domain that recognizes a second tumor antigen.

In some embodiments, the genetically engineered EPX+ eosinophil promotes the anti-tumor activity of the genetically engineered CD3− CD56+ natural killer cell.

In some embodiments, the genetically engineered CD11b+ CD14+ macrophage promotes the anti-tumor activity of the genetically engineered CD3− CD56+ natural killer cell.

In some embodiments, the genetically engineered CD11b+ CD15+ neutrophil secretes inflammatory cytokines after co-culture with tumor cells expressing GD2.

In some embodiments, the solid tumor expresses GD2. In some embodiments, the solid tumor is a neuroblastoma tumor, a melanoma tumor, a glioma tumor, a sarcoma tumor, a lung cancer tumor, a breast cancer tumor, or a pancreatic tumor.

Melanoma tumors can include, but are not limited to, superficial spreading melanoma, nodular melanoma, lentigo malignant melanoma, amelanotic melanoma, acral lentiginous melanoma, mucosal melanoma, melanoma of the eye, and desmoplastic melanoma.

Glioma tumors can include, but are not limited to, astrocytoma, ependymoma, glioblastoma, and oligodendroglioma.

Sarcoma tumors can include, but are not limited to, osteosarcoma, dermatofibrosarcoma protuberans (DFSP), fibrosarcoma (fibroblastic sarcoma), chondrosarcoma, Ewing's sarcoma, rhabdomyosarcoma, liposarcoma, synovial sarcoma, pleomorphic sarcoma, gastrointestinal stromal tumor, Kaposi's sarcoma, leiomyosarcoma, and angiosarcoma.

Lung cancer tumors can include, but are not limited to, adenocarcinoma, squamous cell carcinoma, large cell carcinoma, small cell lung cancer, lung carcinoid tumor, and adenoid cystic carcinomas. Lung cancer tumors can also include cancers that start in other organs (such as breast, pancreas, kidney, skin, or brain) and spread to the lungs.

Breast cancer tumors can include, but are not limited to, adenocarcinoma, ductal carcinoma in situ (DCIS), invasive ductal carcinoma (IDC), medullary carcinoma, mucinous carcinoma, tubular carcinoma, papillary carcinoma, cribriform carcinoma, lobular carcinoma, inflammatory breast cancer, phyllodes tumor of the breast, angiosarcomas, Paget's disease, metastatic breast cancer, triple negative breast cancer, hormone receptor-positive or -negative breast cancer.

Pancreatic cancer tumors can include, but are not limited to, exocrine pancreatic cancer (e.g., adenocarcinoma, squamous cell carcinoma, adenosquamous carcinoma, colloid carcinoma) and pancreatic neuroendocrine tumors.

In some embodiments, the genetically engineered CD11b+ CD14+ macrophage, genetically engineered CD3− CD56+ natural killer cell, genetically engineered EPX+ eosinophil, and/or genetically engineered CD11b+ CD15+ neutrophil are autologous or allogenic to the subject.

Any appropriate dosage can be used for a therapeutic method provided herein. The cell dose will depend on the extent and severity of the solid tumor but a suitable range is from about 1×10⁸ cells/patient to about 1×10¹⁰ cells/patient per dose. In some cases, HPCs obtained as described herein are co-administered to a subject with other cell types including, for example, macrophages and neutrophils.

After administering the cells into the subject, the effect of the treatment method can be evaluated, if desired, using any appropriate method known to practitioners in the art. The treatment can be repeated as needed or required. Following treatment according to the methods provided herein, the treated subject can be monitored for any positive or negative changes in solid tumor being treated.

Administration of a therapeutically effective amount of CAR macrophages, CAR natural killer cells, CAR eosinophils, CAR neutrophils, or combinations thereof into the recipient subject is generally effected using methods well known in the art, and usually involves directly injecting or otherwise introducing a therapeutically effective dose of CAR macrophages, CAR natural killer cells, CAR eosinophils, CAR neutrophils, or combinations thereof into the subject using clinical tools known to those skilled in the art (e.g., U.S. Pat. Nos. 6,447,765; 6,383,481; 6,143,292; and 6,326,198). For example, introduction of CAR macrophages, CAR natural killer cells, CAR eosinophils, CAR neutrophils, or combinations thereof of this invention can be injected locally or systemically via intravascular administration, such as intravenous, intramuscular, or intra-arterial administration, intraperitoneal administration, and the like. Cells can be injected into an infusion bag (e.g., Fenwal infusion bag (Fenwal, Inc.)) using sterile syringes or other sterile transfer mechanisms. The cells can then be immediately infused via IV administration over a period of time, such as 15 minutes, into a free flow IV line into the patient. In some embodiments, additional reagents such as buffers or salts are provided to the recipient subject concurrently with the cells.

Various exemplary embodiments of compositions and methods according to this invention are now described in the following non-limiting Examples. The Examples are offered for illustrative purposes only and are not intended to limit the scope of this invention in any way. Indeed, various modifications of the invention in addition to those shown and described herein will become apparent to those skilled in the art from the foregoing description and the following examples and fall within the scope of the appended claims.

### EXAMPLES

Materials and Methods

CAR constructs. The anti-disialoganglioside (GD2) CAR transgene (Pule et al., 2005, Molecular Therapy, 12(5): 933-941). CAR1, CARa, or CARb transgenes were cloned into AAVS1-DEST vector and knock-in into AAVS1 alleles by using CRIPR/Cas9 gene editing technology (Oceguera-Yanez et al., 2016, Methods 101:43-55). The sequences are shown in Table 3.

TABLE 3
CAR constructs

| CAR | |
|---|---|
| Construct | Nucleic Acid/Amino Acid Sequence |
| CARa | ATGGCCCTGCCTGTGACAGCCCTGCTGCTGCCTCTGGCTCTGCTGCTGC |
| | ATGCCGCTAGACCCCAGGTGAAACTGCAGCAGTCAGGACCTGAACTGGT |
| | GAAGCCTGGGGCTTCAGTGAAGATATCCTGCAAGACTTCTGGATACAAA |
| | TTCACTGAATACACCATGCACTGGGTGAAGCAGAGCCATGGAAAGAGCC |
| | TTGAGTGGATTGGAGGTATTAATCCTAACAATGGTGGTACTAACTACAA |
| | GCAGAAGTTCAAGGGCAAGGCCACATTGACTGTAGACAAGTCCTCCAGC |
| | ACAGCCTACATGGAGCTCCGCAGCCTGACATCTGAGGATTCTGCAGTCT |
| | ATTACTGTGCAAGAGATACTACGGTCCCGTTTGCTTACTGGGTCCAAGG |
| | GACCACGGTCACCGTCTCCTCAGGTGGAGGCGGTTCAGGCGGAGGTGGC |
| | TCTGGCGGTGGCGGATCGGACATCGAGCTCACTCAGTCTCCAGCAATCA |
| | TGTCTGCATCTCCAGGGGAGAAGGTCACCATGACCTGCAGTGGCAGCTC |
| | AAGTATAAGTTACATGCACTGGTACCAGCAGAAGCCTGTCACCTCCCCC |
| | AAAAGATGGATTTATGACACATCCAAACTGGCTTCTGGAGTCCCTGCTC |
| | GCTTCAGTGGCAGTGGGTCTGGGACCTCTTATTCTCTCACAATCAGCAG |
| | CATGGAGGCTGTAGATGCTGCCACTTATTACTGCCATCAGCGGAGTAGT |
| | TACCCGCTCACGTTCGGTGCTGGGACACAGTTGGAAATAAAACGGGAAC |
| | AAAAACTTATTTCTGAAGAAGATCTGACCACGACGCCAGCGCCGCGACC |
| | ACCAACACCGGCGCCCACCATCGCGTCGCAGCCCCTGTCCCTGCGCCCA |
| | GAGGCGAGTCGGCCAGCGGCGGGGGGCGCAGTGCACACGAGGGGGCTGG |
| | ACTTCGCCTGTGATATCTACATCTGGGCGCCCTTGGCCGGGACTTGTGG |
| | GGTCCTTCTCCTGTCACTGGTTATCACCCTTTACTGCGGCTCAAAACGG |
| | GGCAGAAAGAAACTCCTGTATATATTCAAACAACCATTTATGAGACCAG |
| | TACAAACTACTCAAGAGGAAGATGGCTGTAGCTGCCGATTTCCAGAAGA |
| | AGAAGAAGGAGGATGTGAACTGGGCTCAAGAGTGAAGTTCAGCAGGAGC |
| | GCAGACGCCCCCGCGTACAAGCAGGGCCAGAACCAGCTCTATAACGAGC |
| | TCAATCTAGGACGAAGAGAGGAGTACGATGTTTTGGACAAGAGACGTGG |
| | CCGGGACCCTGAGATGGGGGGAAAGCCGAGAAGGAAGAACCCTCAGGAA |
| | GGCCTGTACAATGAACTGCAGAAAGATAAGATGGCGGAGGCCTACAGTG |
| | AGATTGGGATGAAAGGCGAGCGCCGGAGGGGCAAGGGGCACGATGGCCT |
| | TTACCAGGGTCTCAGTACAGCCACCAAGGACACCTACGACGCCCTTCAC |
| | ATGCAGGCCCTGCCCCCTCGCTGA (SEQ ID NO: 1) |
| CARa | MALPVTALLLPLALLLHAARPQVKLQQSGPELVKPGASVKISCKTSGYK |
| | FTEYTMHWVKQSHGKSLEWIGGINPNNGGTNYKQKFKGKATLTVDKSSS |
| | TAYMELRSLTSEDSAVYYCARDTTVPFAYWVQGTTVTVSSGGGGSGGGG |
| | SGGGGSDIELTQSPAIMSASPGEKVTMTCSGSSSISYMHWYQQKPVTSP |
| | KRWIYDTSKLASGVPARFSGSGSGTSYSLTISSMEAVDAATYYCHQRSS |
| | YPLTFGAGTQLEIKREQKLISEEDLTTTPAPRPPTPAPTIASQPLSLRP |
| | EASRPAAGGAVHTRGLDFACDIYIWAPLAGTCGVLLLSLVITLYCGSKR |
| | GRKKLLYIFKQPFMRPVQTTQEEDGCSCRFPEEEEGGCELGSRVKFSRS |
| | ADAPAYKQGQNQLYNELNLGRREEYDVLDKRRGRDPEMGGKPRRKNPQE |
| | GLYNELQKDKMAEAYSEIGMKGERRRGKGHDGLYQGLSTATKDTYDALH |
| | MQALPPR (SEQ ID NO: 5) |
| CARb | ATGGAGTTTGGGCTGAGCTGGCTTTTTCTTGTGGCTATTTTAAAAGGTG |
| | TCCAGTGCTCTAGAGATATTTTGCTGACCCAAACTCCACTCTCCCTGCC |
| | TGTCAGTCTTGGAGATCAAGCCTCCATCTCTTGCAGATCTAGTCAGAGT |
| | CTTGTACACCGTAATGGAAACACCTATTTACATTGGTACCTGCAGAAGC |
| | CAGGCCAGTCTCCAAAGCTCCTGATTCACAAAGTTTCCAACCGATTTTC |
| | TGGGGTCCCAGACAGGTTCAGTGGCAGTGGATCAGGGACAGATTTCACA |
| | CTCAAGATCAGCAGAGTGGAGGCTGAGGATCTGGGAGTTTATTTCTGTT |
| | CTCAAAGTACACATGTTCCTCCGCTCACGTTCGGTGCTGGGACCAAGCT |
| | GGAGCTGAAACGGGCTGATGCTGCACCAACTGTATCCATCTTCCCAGGC |
| | TCGGGCGGTGGTGGGTCGGGTGGCGAGGTGAAGCTTCAGCAGTCTGGAC |
| | CTAGCCTGGTGGAGCCTGGCGCTTCAGTGATGATATCCTGCAAGGCTTC |
| | TGGTTCCTCATTCACTGGCTACAACATGAACTGGGTGAGGCAGAACATT |
| | GGAAAGAGCCTTGAATGGATTGGAGCTATTGATCCTTACTATGGTGGAA |
| | CTAGCTACAACCAGAAGTTCAAGGGCAGGGCCACATTGACTGTAGACAA |
| | ATCGTCCAGCACAGCCTACATGCACCTCAAGAGCCTGACATCTGAGGAC |
| | TCTGCAGTCTATTACTGTGTAAGCGGAATGGAGTACTGGGGTCAAGGAA |
| | CCTCAGTCACCGTCTCCTCAGCCAAAACGACACCCCCATCAGTCTATGG |
| | AAGGGTCACCGTCTCTTCAGCGGAGCCCAAATCTTGTGACAAAACTCAC |
| | ACATGCCCACCGTGCCCGGATCCCAAATTTTGGGTGCTGGTGGTGGTTG |
| | GTGGAGTCCTGGCTTGCTATAGCTTGCTAGTAACAGTGGCCTTTATTAT |
| | TTTCTGGGTGAGGAGTAAGAGGAGCAGGCTCCTGCACAGTGACTACATG |
| | AACATGACTCCCCGCCGCCCCGGGCCCACCCGCAAGCATTACCAGCCCT |
| | ATGCCCCACCACGCGACTTCGCAGCCTATCGCTCCAGGGACCAGAGGCT |
| | GCCCCCCGATGCCCACAAGCCCCCTGGGGGAGGCAGTTTCCGGACCCCC |
| | ATCCAAGAGGAGCAGGCCGACGCCCACTCCACCCTGGCCAAGATCAGAG |
| | TGAAGTTCAGCAGGAGCGCAGACGCCCCCGCGTACCAGCAGGGCCAGAA |
| | CCAGCTCTATAACGAGCTCAATCTAGGACGAAGAGAGGAGTACGATGTT |
| | TTGGACAAGAGACGTGGCCGGGACCCTGAGATGGGGGGAAAGCCGAGAA |
| | GGAAGAACCCTCAGGAAGGCCTGTACAATGAACTGCAGAAAGATAAGAT |
| | GGCGGAGGCCTACAGTGAGATTGGGATGAAAGGCGAGCGCCGGAGGGGC |
| | AAGGGGCACGATGGCCTTTACCAGGGTCTCAGTACAGCCACCAAGGACA |
| | CCTACGACGCCCTTCACATGCAGGCCCTGCCCCCTCGCTAA (SEQ ID |
| | NO: 2) |
| CARb | MEFGLSWLFLVAILKGVQCSRDILLTQTPLSLPVSLGDQASISCRSSQS |
| | LVHRNGNTYLHWYLQKPGQSPKLLIHKVSNRFSGVPDRFSGSGSGTDFT |
| | LKISRVEAEDLGVYFCSQSTHVPPLTFGAGTKLELKRADAAPTVSIFPG |
| | SGGGGSGGEVKLQQSGPSLVEPGASVMISCKASGSSFTGYNMNWVRQNI |
| | GKSLEWIGAIDPYYGGTSYNQKFKGRATLTVDKSSSTAYMHLKSLTSED |
| | SAVYYCVSGMEYWGQGTSVTVSSAKTTPPSVYGRVTVSSAEPKSCDKTH |
| | TCPPCPDPKFWVLVVVGGVLACYSLLVTVAFIIFWVRSKRSRLLHSDYM |
| | NMTPRRPGPTRKHYQPYAPPRDFAAYRSRDQRLPPDAHKPPGGGSFRTP |
| | IQEEQADAHSTLAKIRVKFSRSADAPAYQQGQNQLYNELNLGRREEYDV |
| | LDKRRGRDPEMGGKPRRKNPQEGLYNELQKDKMAEAYSEIGMKGERRRG |
| | KGHDGLYQGLSTATKDTYDALHMQALPPRQPDAIHSSGAGCLSEGGGWC |
| | GQCPGSQIPLRSFSLCQKLWGHHEAP-ASDFWLIKEIYFHCNSVLEFFV |
| | SLTRKDIWEGKSFKTSE-VFGLEFGNICPYAGCHEQRLAIKRSSVYETA |
| | PCCPFLIPKSLDLRLDFFYILFCVIFFFNIPKIFLTCFTSQIFPPLLTT |
| | PSHSCPSSLMEI (SEQ ID NO: 6) |
| CAR1 | ATGGAGTTCGGCCTGTCTTGGCTGTTTCTGGTGGCCATCCTGAAGGGCG |
| | TGCAGTGCAGCAGAGACATCCTGCTGACACAGACCCCACTGAGCCTGCC |
| | CGTGAGCCTGGGCGATCAGGCCTCTATCAGCTGTCGCAGCTCCCAGTCT |
| | CTGGTGCACCGGAACGGCAATACCTACCTGCACTGGTATCTGCAGAAGC |
| | CAGGCCAGAGCCCCAAGCTGCTGATCCACAAGGTGTCTAACAGATTCAG |
| | CGGCGTGCCTGACAGGTTTTCCGGCTCTGGCAGCGGCACAGATTTCACC |
| | CTGAAGATCTCCAGAGTGGAGGCCGAGGACCTGGGCGTGTACTTCTGCT |
| | CCCAGTCTACACACGTGCCCCCTCTGACATTTGGCGCCGGCACCAAGCT |
| | GGAGCTGAAGAGGGCAGATGCAGCACCTACCGTGAGCATCTTTCCAGGA |
| | TCCGGAGGAGGAGGATCTGGAGGAGAGGTGAAGCTGCAGCAGTCTGGAC |
| | CAAGCCTGGTGGAGCCTGGAGCATCCGTGATGATCTCTTGTAAGGCCAG |
| | CGGCTCTAGCTTCACAGGCTACAACATGAATTGGGTGCGCCAGAACATC |
| | GGCAAGAGCCTGGAGTGGATCGGCGCCATCGACCCCTACTATGGCGGCA |
| | CCTCCTACAATCAGAAGTTTAAGGGCCGGGCCACACTGACCGTGGACAA |
| | GTCCTCTAGCACAGCCTATATGCACCTGAAGTCCCTGACCTCTGAGGAT |
| | AGCGCCGTGTACTATTGCGTGAGCGGCATGGAGTACTGGGGCCAGGGCA |
| | CATCCGTGACCGTGTCCTCTGCCAAGACCACACCACCCAGCGTGTATGG |
| | CCGCGTGACAGTGAGCTCCGCCTTCGTGCCCGTGTTCCTGCCTGCCAAG |
| | CCAACCACAACCCCTGCACCAAGGCCTCCAACACCAGCACCTACCATCG |
| | CATCCCAGCCACTGTCTCTGAGGCCAGAGGCATGTAGGCCTGCAGCAGG |
| | CGGCGCCGTGCACACCAGGGGCCTGGACTTCGCCTGCGATATCTACATC |
| | TGGGCACCACTGGCAGGAACATGTGGCGTGCTGCTGCTGTCCCTGGTCA |
| | TCACCAGATCCAAGCGGAGCCGGCTGCTGCACTCTGACTATATGAACAT |
| | GACACCACGGAGACCAGGACCAACCAGGAAGCACTACCAGCCTTATGCA |
| | CCACCTAGGGATTTTGCAGCATACCGGTCCAAGAGAGGCAGGAAGAAGC |
| | TGCTGTATATCTTCAAGCAGCCTTTTATGAGACCAGTGCAGACAACCCA |
| | GGAGGAGGACGGCTGCAGCTGTAGGTTCCCAGAAGAGGAGGAGGGAGGA |
| | TGCGAGCTGAGAGTGAAGTTTAGCAGGTCCGCCGATGCACCAGCATACC |
| | AGCAGGGACAGAATCAGCTGTATAACGAGCTGAATCTGGGCAGGCGCGA |
| | GGAGTACGACGTGCTGGATAAGAGGAGAGGAAGGGATCCAGAGATGGGA |
| | GGCAAGCCAAGGCGCAAGAACCCCCAGGAGGGCCTGTACAATGAGCTGC |
| | AGAAGGACAAGATGGCCGAGGCCTATTCCGAGATCGGCATGAAGGGAGA |
| | GCGGAGAAGGGGCAAGGGACACGATGGCCTGTACCAGGGCCTGAGCACA |
| | GCCACCAAGGACACCTATGATGCACTGCACATGCAGGCCCTGCCACCAA |
| | GGTGA (SEQ ID NO: 3) |
| CAR1 | MEFGLSWLFLVAILKGVQCSRDILLTQTPLSLPVSLGDQASISCRSSQS |
| | LVHRNGNTYLHWYLQKPGQSPKLLIHKVSNRFSGVPDRFSGSGSGTDFT |
| | LKISRVEAEDLGVYFCSQSTHVPPLTFGAGTKLELKRADAAPTVSIFPG |
| | SGGGGSGGEVKLQQSGPSLVEPGASVMISCKASGSSFTGYNMNWVRQNI |
| | GKSLEWIGAIDPYYGGTSYNQKFKGRATLTVDKSSSTAYMHLKSLTSED |
| | SAVYYCVSGMEYWGQGTSVTVSSAKTTPPSVYGRVTVSSAFVPVFLPAK |
| | PTTTPAPRPPTPAPTIASQPLSLRPEACRPAAGGAVHTRGLDFACDIYI |
| | WAPLAGTCGVLLLSLVITRSKRSRLLHSDYMNMTPRRPGPTRKHYQPYA |
| | PPRDFAAYRSKRGRKKLLYIFKQPFMRPVQTTQEEDGCSCRFPEEEEGG |
| | CELRVKFSRSADAPAYQQGQNQLYNELNLGRREEYDVLDKRRGRDPEMG |
| | GKPRRKNPQEGLYNELQKDKMAEAYSEIGMKGERRRGKGHDGLYQGLST |
| | ATKDTYDALHMQALPPR (SEQ ID NO: 7) |

Cell Culture. Wild-type H9 and BM9 human pluripotent stem cells (hPSCs) were obtained from WiCell (Madison, WI). GD2-CAR human pluripotent stem cells (GD2-CAR hPSCs) were generated with using gene editing technology. Wild-type and GD2-CAR hPSCs were cultured on Matrigel-coated tissue culture plates in E8 medium (STEMCELL Technologies).

Generation of GD2-CAR macrophages. The methods described herein can be used to produce CAR macrophages that target a variety of antigens, including GD2.

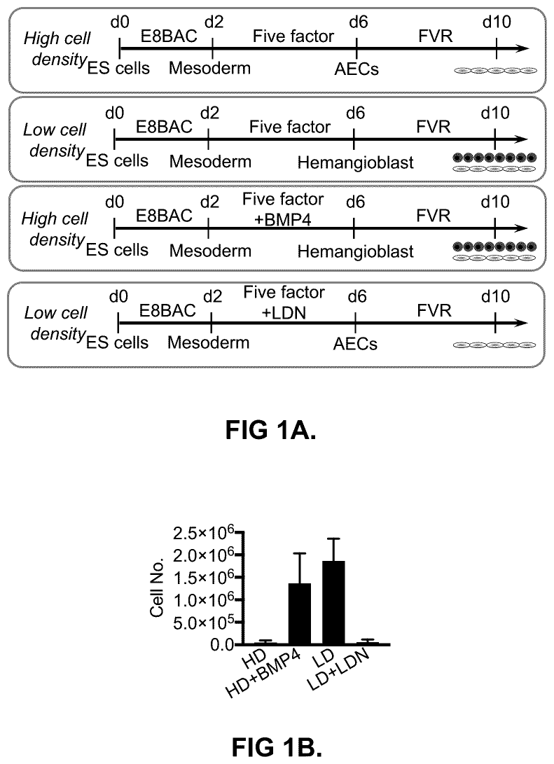

The derivation of hematopoietic progenitor/stem cells and macrophages were disclosed in U.S. Patent Application Publication No. 2020-0080059, the contents of which are incorporated herein by reference. Briefly, human pluripotent stem cells were differentiated into hematopoietic progenitor/stem cells and macrophage by endothelial to hematopoietic transition in both low density (e.g., a cell culture seeded at a density of between about 6×10³ cells/cm² and about 6×10⁴ cells/cm²) and high-density cultures (e.g., a cell culture seeded at a density of between above about 6×10⁴ cells/cm² and about 3×10⁵ cells/cm²). For seeding cells at low density, cell-cell contact was reduced. For high-density cultures, adding BMP4 promoted hematopoiesis (FIG. 1A and FIG. 1B).

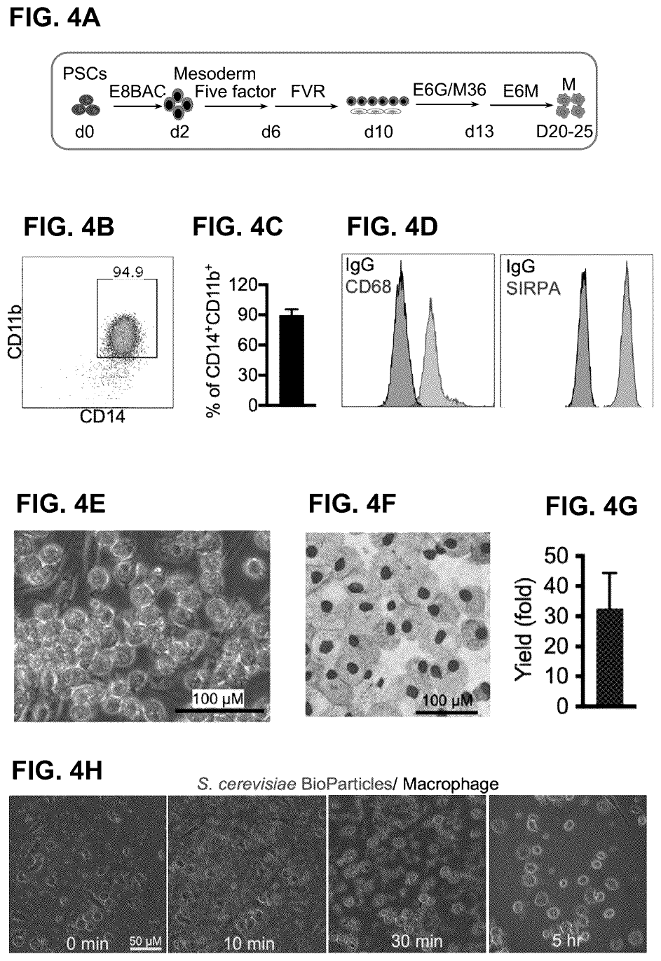

The same differentiation media was used for both low density and high-density conditions. Pluripotent stem cells were cultured in a chemically defined culture medium comprising or consisting essentially of DMEM/F12 culture medium, L-ascorbic acid-2-phosphate magnesium, sodium selenium, human FGF2, insulin, NaHC03, transferrin, TGFβ1, BMP4, Activin-A, and CHIR99021 (“E8BAC medium”) for two days. The medium was then changed to “five factor” medium until day 6. At day 6, hematopoietic cells were observed in low density culture. At day 10, more hematopoietic cells were observed and they expressed hematopoietic progenitor cell markers, CD34 and CD45. From day 6 to day 10 the culture was maintained in FVR medium. Hematopoietic cells could be cryopreserved at day 10 (FIG. 1A). The hematopoietic cells were cultured in E6G (or M36) and E6M medium to differentiate into macrophage (FIG. 4A).

Generation of GD2-CAR natural killer cells with serum-free and feeder-free NK cell differentiation protocol: The PSCs were plated on a vitronectin- or Matrigel-coated plate at high density (1.1×10⁵ cells/cm²) in E8BAC medium for 2 days. The mesoderm cells were passaged and seeded on a new vitronectin- or Matrigel-coated plate at low density (0.2-1.1×10⁵ cells/cm²). From day 2-3, E6T medium was used. From day 3-8, “five factor” medium was used. From day 8 to day 12, FVR medium was used. From day 12-15, S-B medium was used to further expand the hematopoietic progenitors. From day 15, the NK medium was used for another 15-20 days. E6T medium is not required for some cell lines. For some cell lines, day 12 hematopoietic progenitors can be directly used for NK cell differentiation.

Generation of GD2-CAR eosinophils with serum-free and feeder-free eosinophil differentiation protocol: The PSCs were plated on a vitronectin- or Matrigel-coated plate at high density (1.1×10⁵ cells/cm²) in E8BAC medium for 2 days. The mesoderm cells were passaged and seeded on a new vitronectin- or Matrigel-coated plate at low density (0.7-1.1×10⁵ cells/cm²). From day 2-6, “five factor” medium was used. From day 6 to day 10, FVR medium was used. From day 10, the EM medium was used for another 15-20 days.

Generation of GD2-CAR neutrophils. The methods described herein can be used to produce CAR neutrophils that target a variety of antigens, including GD2.

GD2-CAR neutrophils were prepared from pluripotent stem cells (PSCs) engineered to express CARb, using the methods of U.S. Patent Application Publication No. 20200385676 (programming neutrophils using ETV2 mmRNA), the contents of which are incorporated herein by reference, and those described herein to produce CAR-macrophages. The GD2-CAR cell lines were used to generate neutrophils in serum-free, xenogen-free, and feeder-free conditions using ETV2 modified mRNA-based differentiation system developed by the inventors.

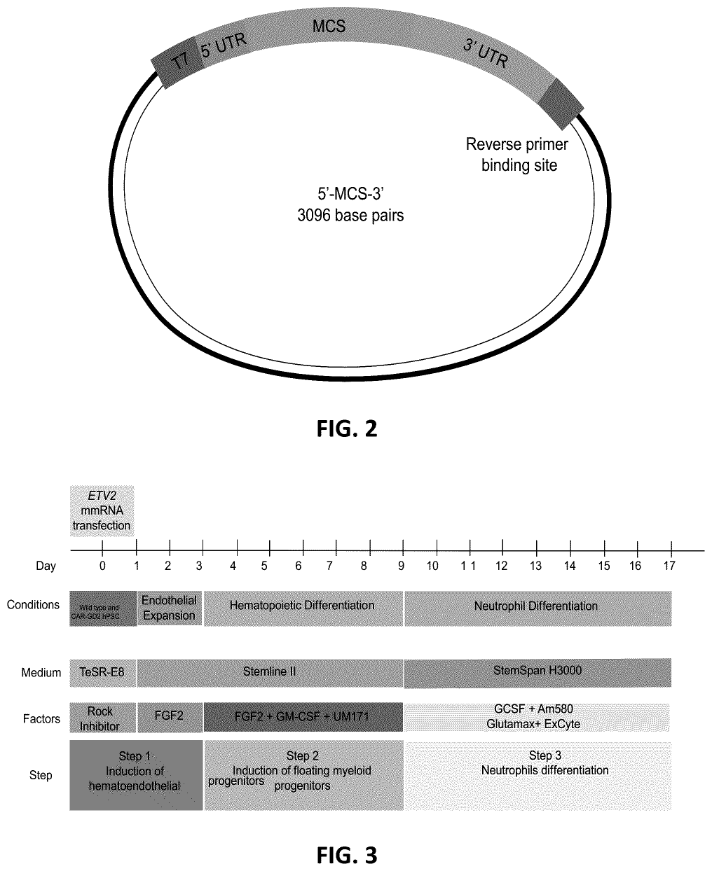

mmRNA synthesis and transfection. Human ETV2 transcript variant 1 (NM_014209.3) was cloned into a 5′-MCS-10 construct as described previously (Suknuntha et al., 2018, Stem Cell Reviews and Reports 14) (FIG. 2). To generate IVT templates with a 180-A tract, a reverse primer containing 180 T base pairs and an ATCGGTGCGGGCCTCTTCGCTA (SEQ ID NO.4) forward primer including T7 promoter were used in a PCR reaction. All PCR reactions were carried out using Phusion (Thermo Fisher Scientific). The mmRNA was synthesized using the MEGAscript T7 Kit (Ambion, Austin, TX), using a custom ribonucleoside cocktail comprised of 3′-O-Me-m7G(5′)ppp(5′)G ARCA cap analog, pseudouridine triphosphate (TriLink BioTechnologies, San Diego, CA), ATP, guanosine triphosphate, and cytidine triphosphate. The synthesis reactions were set up according to the manufacturer's instructions. Reactions were incubated for 2 h at 37° C. and treated with DNAse. RNA was purified using a PureLink RNA Mini Kit (Thermo Fisher Scientific) and adjusted with RNase-free water to 100 ng/L working concentration before being stored at −80° C. Undifferentiated hPSCs were transfected with using TransIT-mRNA reagent in E8 medium containing ROCK inhibitor (Suknuntha et al., 2018). In brief, for transfection, single-cell suspension was prepared using HyQtase (Thermo Fisher Scientific). Per one well of transfection, a total of 2×10⁵ cells in 1 mL complete E8 medium with 10 μM ROCK inhibitor (STEMCELL Technologies) were plated into a collagen IV-coated 6-well plate; 30 mins later, a mixture of 300 ng ETV2: TransIT-mRNA (Mirus Bio, Madison, WI) was added to each well according to the manufacturer's instructions.

Generation of GD2-CAR neutrophils with serum-free, feeder-free, and xeno-free neutrophil differentiation protocol from hPSCs. The day after transfection (day 1), the medium was changed with 1 mL of Stemline II (Sigma) supplemented with 20 ng/mL of human FGF2 (PeproTech). On day 2, 1 mL of the same medium was added. On day 3, the medium was changed and 1 mL of Stemline II supplemented with FGF2 (20 ng/mL), GM-CSF (25 ng/mL) (PeproTech), and UM171 (50 nM; Xcess Biosciences) were added. This medium was added daily up to days 8. On day 9, floating cells were gently harvested and used for terminal neutrophil differentiation. To induce neutrophil terminal differentiation, floating cells were cultured in StemSpanH300 medium (STEMCELL Technologies), supplemented with GlutaMAX 100× (Thermo Fisher Scientific), ExCyte 0.2% (Merck Millipore), human G-CSF (150 ng/mL; Amgen), Am580 retinoic acid agonist 2.5 μM (Sigma-Aldrich), and gentamycin (1,000×) (Life Technologies) at 5×10⁵ cells/mL density. After 4 days, 2 mL of the same medium with all components and cytokines was added on the top of existing culture. Mature neutrophils were gently harvested from the supernatant after 8 days of culture, leaving the adherent macrophages, and filtered through a 70-μM mesh (Falcon, Life Sciences) before analysis (FIG. 3).

Flow Cytometry. To analyze cell surface markers, 5×10⁵ cells were stained in fluorescence-activated cell sorting buffer with the appropriate antibodies (Table 4). Ghost Dye (Tonbo Biosciences, San Diego, CA) was used to analyze the live cell population. Cells were analyzed using a MACSQuant Analyzer 10 (Miltenyi Biotec, San Diego, CA) or Thermo Fisher Scientific Attune FlowJo software (Tree Star, Ashland, OR).

TABLE 4
Antibodies

| ANTIBODIES | SOURCE |
|---|---|
| Mouse anti-human CD43-BV421 | BD Horizon ™ |
| Mouse anti-human CD45 APC | BD Pharmingen ™ |
| Mouse anti-human CD11b FITC | BD Pharmingen ™ |
| Mouse anti-human CD16 PE | BD Pharmingen ™ |
| Anti-human CD3-PE | BD Biosciences |
| Anti-human CD56-APC | BD Biosciences |
| Anti-EPX | ABCAM INC |

Wright-Giemsa Staining. To assess the morphology of cells within colonies, cells were fixed on glass slides using a Cytospin centrifuge (Cytospin 2; Thermo Shandon), stained with Wright-Giemsa solution (Sigma-Aldrich), and then observed under a light microscope (Olympus, Tokyo).

In vitro Cytotoxicity Assay. WM266-4 LUC2 GFP (human melanoma cell line), CHLA-20, CHLA-20_AAVS1-AkaLuc-EGFP (human neuroblastoma cell line), SK-BR3 LUC2 GFP (human breast adenocarcinoma cancer cell) and SKOV3 LUC2 GFP cells (human ovarian cancer cell line) were maintained in tumor specialized media (Sigma-Aldrich) containing 80% MCDB-153, 20% Leibovitz's L-15, 1.68 mM CaCl₂) and 2% FBS. To access the cytotoxicity, hPSC derived wild-type and CAR-GD2 neutrophils cells were incubated with target tumor cells (2,000 cells/well) for 4 hours at 37° C., at effector:target (E:T) ratios of 1:1, 2:1, 5:1, and 10:1, in a final volume of 200 μl, in a 96 well plate. Target cells were used for the maximal lysis with Pierce™ IP Lysis Buffer (ThermoFisher). VivoGlo™ Luciferin substrate (100 ug/well, Promega) was added, and luminescence was measured immediately after 5 mins incubation. Specific cell lysis was measured by % of specific cell lysis=100×[(Spontaneous cell lysis−Test cell lysis)/(Spontaneous cell lysis−Maximal lysis)].

In vitro Cytotoxicity Assay of macrophage, NK cells, and eosinophils. AkaLuc-GFP was cloned into AAVS1-DEST vector and then integrated into AAVS1 locus of CHLA-20, WM266-4, K562, and Raji cells by using CRISPR-Cas9 technology. Arterial endothelial cells and smooth muscle cells were derived from NOS3-NanoLuc-tdTomato and MYH11-NanoLuc-tdTomato H1 cell lines. To assess cytotoxicity, cells were incubated at indicated effectors:targets (E:T) ratio 20-24 hours in 96-well plate. Luciferase substrate (50-250 μM Tokeoni for AkaLuc from R&D Systems; Nano-Glo for NanoLuc from Promega, 1:1000 dilution) was added to the cell culture and bioluminescence was measured using Promega GloMax plate reader 15 minutes later.

CFSE staining. NK cells were stained with 1 uM CFSE in PBS for 10 mins at 37 C. NK cells were washed with 2% FBS-PBS for 2 times. NK cells were co-cultured with macrophages and tumor cells for 20-24 hours. All the co-culture cells were harvest and stained with CD56-APC and followed by flow cytometric analysis.

Southern Blot. Human iPSCs were transfected with sgRNA, CAS9 protein with GD2-CAR vector and selected by puromycin. Puromycin-resistant cells were clonally expanded and on-targeted clones were selected by southern blot. Southern blot analysis was performed by DIG-labeling hybridization. The external probe is a DIG-labeled fragment that binds to the ApaLI digested fragment of 5′ external region. The internal probe is a DIG-labeled fragment that binds to the EcoRI digested fragment of the puromycin region.

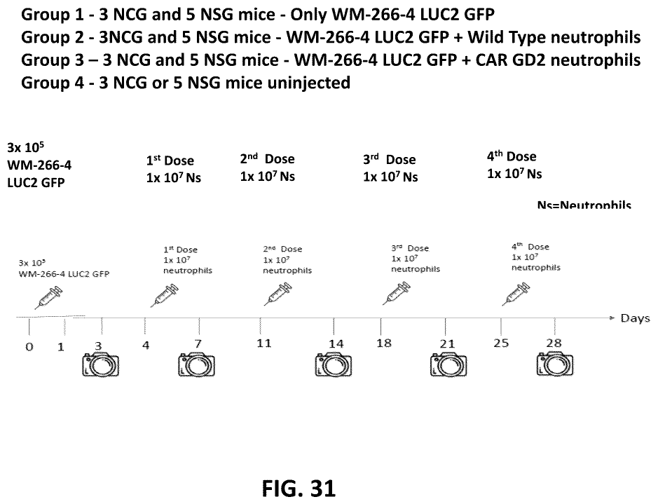

In vivo anti-tumor activity analysis for CAR-N. Mice were inoculated intraperitoneally with 3×10⁵ Luc2-eGFP-expressing WM-266-4 melanoma cells. Melanoma cell engraftment was assessed by IVIS imaging 3 days later for baseline pretreatment reading. On day 4 after melanoma injection, mice were left untreated, or treated with 10⁷ unmodified neutrophils or GD2-CAR-neutrophils injected intraperitoneally every 7 days. Tumor burden was determined by bioluminescent imaging. A schematic of the cytotoxicity model is shown in FIG. 31.

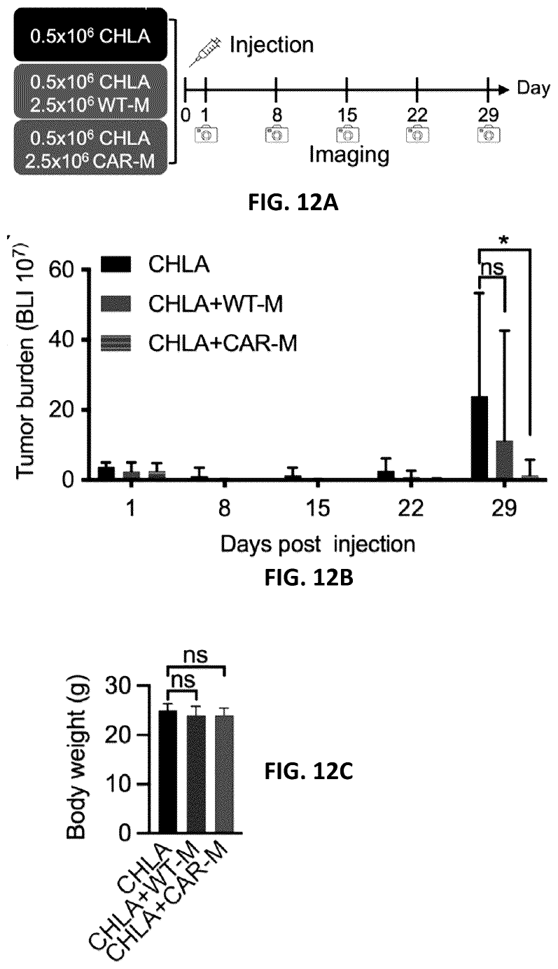

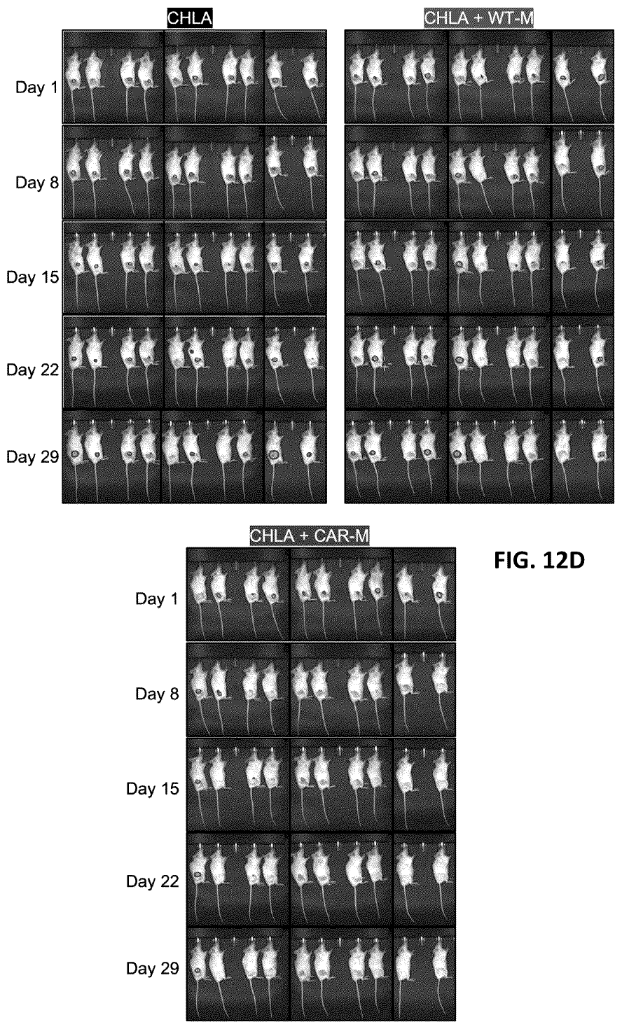

In vivo anti-tumor activity analysis for CAR-M. NOD-scid IL2 Rgammanull (NSG) mice (6- to 8-week-old) were obtained from The Jackson Laboratories. Neuroblastoma cells CHLA20-AkaLuc-GFP and macrophages were injected into the hind flank of the mice. Antitumor effect was monitored by bioluminescent imaging using IVIS imaging system at the indicated time (100 μl 5 mM Tokeoni/mouse). (FIG. 12D).

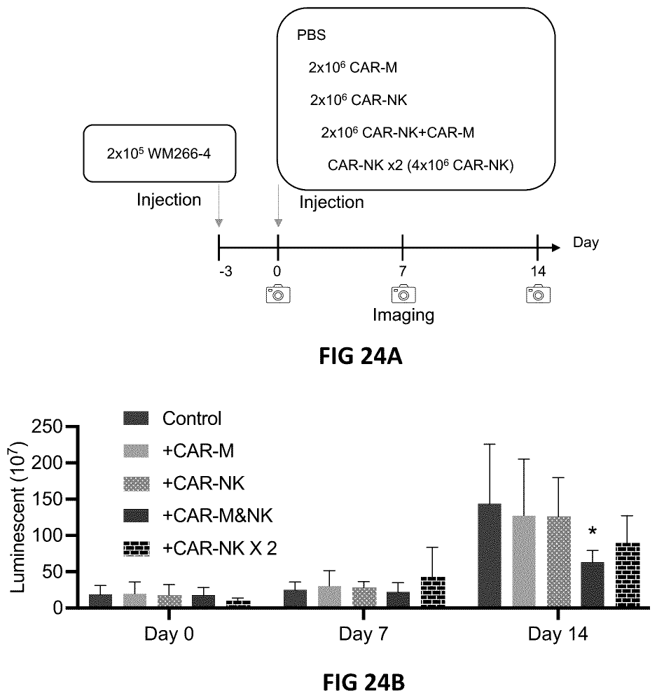

In vivo anti-tumor activity analysis for combination of CAR-M and CAR-NK NOD-scid IL2Rgammanull (NSG) mice (6- to 8-week-old) were obtained from Jackson Lab. WM-266-4-AkaLuc-GFP melanoma cells (2×10⁵/mouse) were injected into the hind flank of the mice. Three days later, 2×10⁶ CAR-M and/or CAR-NK, or 4×10⁶ CAR-NK (CAR-NK×2) were intravenous injected to the mice. Luminescent signals were measured at 0, 7, and 14 days post CAR-M/NK injection. 5-6 mice per group. Antitumor effect was monitored by bioluminescent imaging using IVIS imaging system at the indicated time (100 μl 5 mM Tokeoni/mouse)(shown in FIGS. 24A and 24B).

Gene Ontology (GO) Analysis and Heatmaps. The most highly differentially expressed genes were then subjected to GO analysis. A threshold of unadjusted p-val<=0.05 was used, resulting in 807 and 868 down-regulated and up-regulated genes respectively. Gene set enrichment analysis on the differential expression (DE) genes was performed using GOSeq (Young et al., 2010, Genome Biol 11, R14. 10.1186/gb2010-11-2-r14.) using all Biological Process gene sets in the Gene Ontology downloaded from MSigDB (Subramanian et al., 2005, Proc. Natl. Acad. Sci. USA 102: 15545-15550. 10.1073/pnas.0506580102.). GO ontology terms related to macrophages were chosen for display. For heat mapping, the gene expression data was normalized by computing log(TPM+1) and then for each gene, the Z-score was computed by subtracting the mean and dividing by the standard deviation over all of the samples selected for the heatmap.

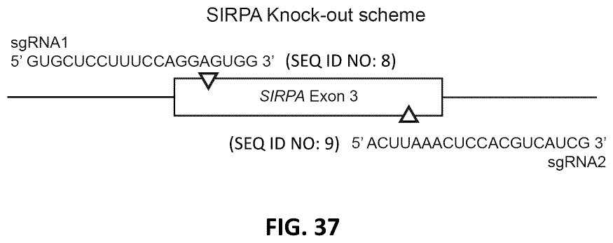

Generation of SIRPa-Knockout. Exon 3 of the SIRPA gene was targeted with two flanking sgRNAs (SEQ ID NO: 8 and SEQ ID NO: 9). The CD47 binding region of SIRPa lies on exon 3, making it an ideal target for a functional SIRPa protein knockout (FIG. 37). Exon 2 of the SIRPa gene was targeted with two flanking sgRNAs CCCUCCUCGCUCCGCAGCCG (SEQ ID NO: 10) and CCGCUGCACUCCCCAAACUG (SEQ ID NO: 11).

### Example 1: Anti-Tumor Activity of GD2-CAR Macrophages In Vitro

To generate macrophages, the hematopoietic cells were further differentiated for another 10-15 days in macrophage differentiation medium (FIG. 4A). The protocol generated more than 90% of CD11b⁺CD14⁺ macrophages, which also expressed CD68 and SIRPa/CD172A (FIG. 4B, FIG. 4C, and FIG. 4D). The macrophages displayed large cell size of a 20-am diameter (as observed by microscopy). The final yield of macrophage is about 30-fold from a starting pluripotent stem cell (FIG. 4G). In addition, they were large in size (20 am diameter) and possessed typical macrophage morphology (FIG. 4E and FIG. 4F). To characterize the phagocytosis function of macrophage, bacteria particles were added to media. Zymosan A S. cerevisiae BioParticles® (Texas Red® conjugate; Life Technologies) were prepared in PBS (10 mg/ml=2×10⁹ particle/ml). 20 μl of particles were added to 2 ml media containing 4×10⁵ macrophage. Phagocytosis was imaged over time. The results revealed that these macrophages were able to uptake bacteria particles (FIG. 4H), demonstrating that they were functional macrophages. GD2-CAR macrophages were differentiated using the macrophage differentiation protocol as described herein.

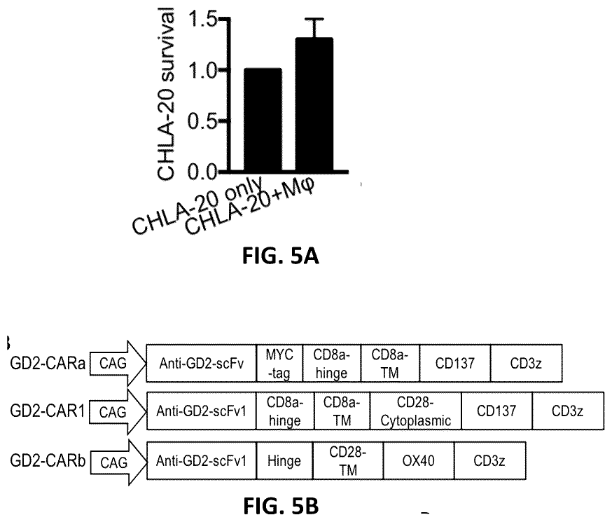

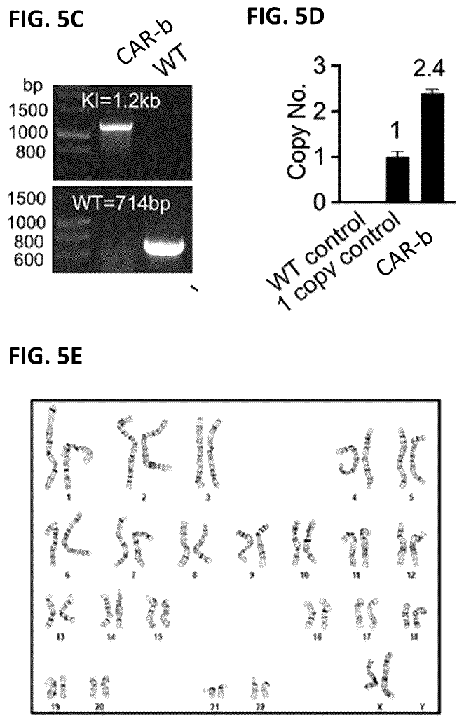

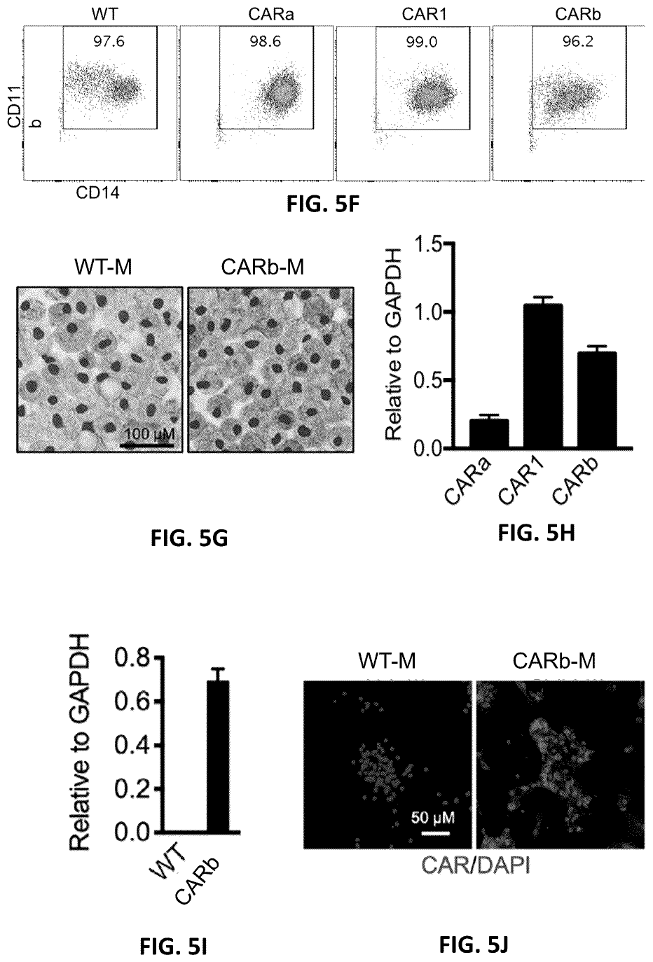

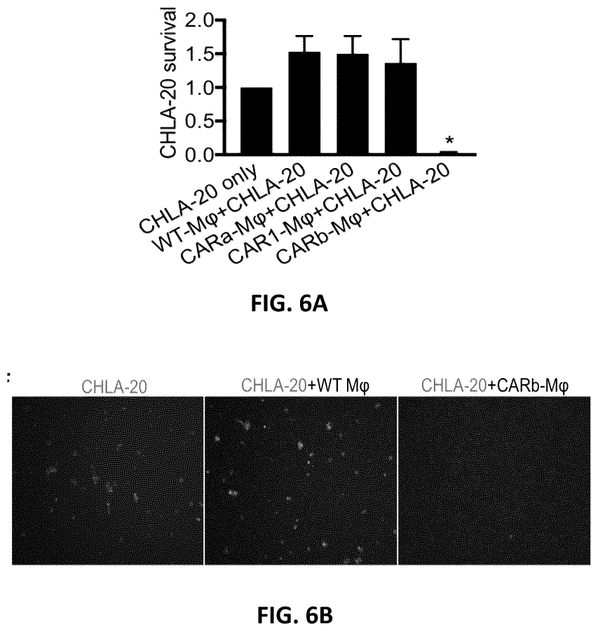

The anti-tumor activity of the GD2-CAR macrophages in vitro was evaluated. Macrophages were co-cultured with CHLA-20 neuroblastoma. However, these macrophages were not able to inhibit the tumor cells (FIG. 5A). Instead, the tumor cell survival was increased by macrophage (FIG. 5A), indicating that the macrophage could be M2 pro-cancer macrophage. In order to reprogram the cells into anti-cancer macrophage, gene editing technology (e.g., CRSPR-CAS9 technology) was applied to knock in GD2-CAR into AAVS1 locus of pluripotent stem cells. In order to reprogram the cells into anti-cancer macrophages, three different GD2-CAR constructs were introduced into the AAVS1 locus of separate H1 and/or H9 pluripotent stem cell lines using genetic engineering (FIG. 5B through FIG. 5E). These cells were then differentiated into macrophages (FIG. 5F), which displayed similar morphology with wild-type macrophages (WT-M) (FIG. 5G). Although all GD2-CARs were expressed as confirmed by qRT-PCR (FIG. 5H and FIG. 5I) and immunostaining of anti-GD2 CAR in CARb-Ms (FIG. 5J), GD2-CARa, and GD2-CAR1 macrophage were not able to reduce the cancer cells (FIG. 6A). In contrast, the cancer cells were almost undetectable when cultured with GD2-CARb-macrophages (FIG. 6A). Imaging and time-lapse results further demonstrated that the green signal from CHLA-20-AkaLuc-GFP cells was diminished by GD2-CARb-macrophages (FIG. 6B).

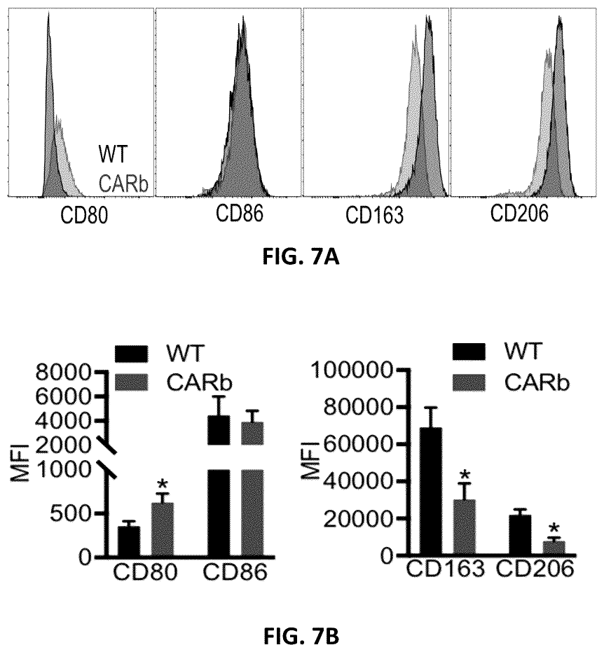

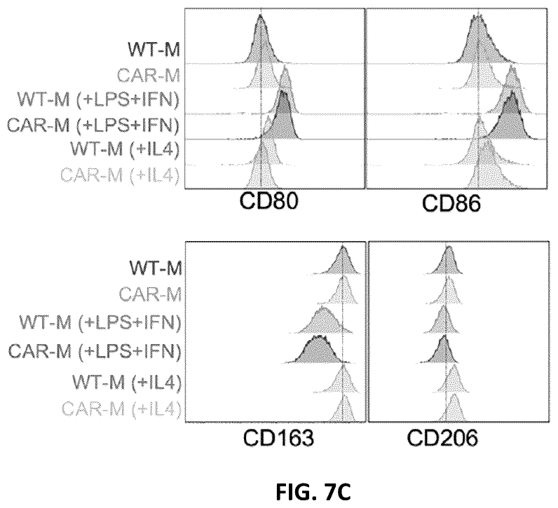

To further characterize the GD2-CARb macrophages, the M1/M2 makers were examined. The results revealed that GD2-CARb macrophages expressed higher CD80 (M1 maker) but lower CD163 and CD206 (M2 marker) (FIG. 7A and FIG. 7B), indicating a M1-like anti-cancer phenotype. In addition, macrophages were treated with lipopolysaccharide (LPS) and interferon γ (IFNγ) to induce M1 polarization or IL-4 to induce M2 polarization. The results revealed that CD80 and CD86 (M1 markers) expression was greatly increased by LPS and IFNγ in both WT-Ms and CAR-Ms (FIG. 7C and FIG. 7D). In addition, LPS and IFNγ decreased CD163 expression (an M2 marker) in CAR-Ms but not WT-Ms (FIG. 7C and FIG. 7D). On the other hand, IL-4 increased CD206 expression (an M2 marker) in both WT-Ms and CAR-Ms (FIG. 7C and FIG. 7D).

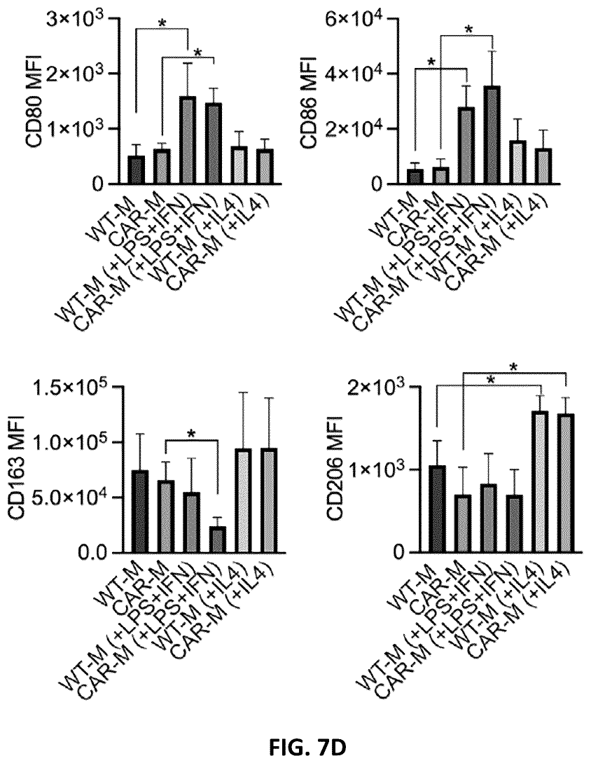

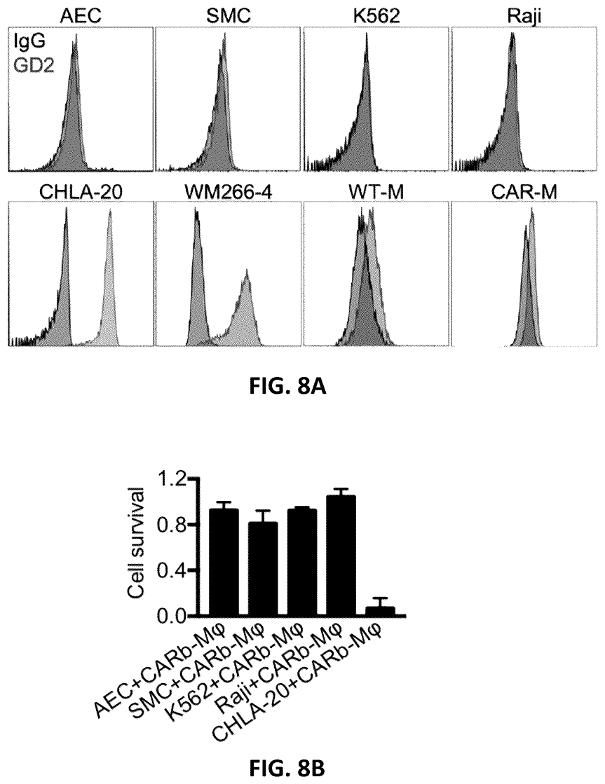

To test the specificity, GD2-CARb macrophages were cultured with cells that are GD2 negative or that do not express GD2 highly, including arterial endothelial cells and smooth muscle cells derived from human NOS3-NanoLuc-tdTomato and MYH11-NanoLuc-tdTomato H1 pluripotent stem cells and K562-AkaLuc-eGFP and Raji-AkaLuc-eGFP cancer cells. The results demonstrated that all the GD2 negative cells were resistance to GD2-CARb-macrophages (FIGS. 8A and 8B), suggesting the antigen specific anti-cancer activity.

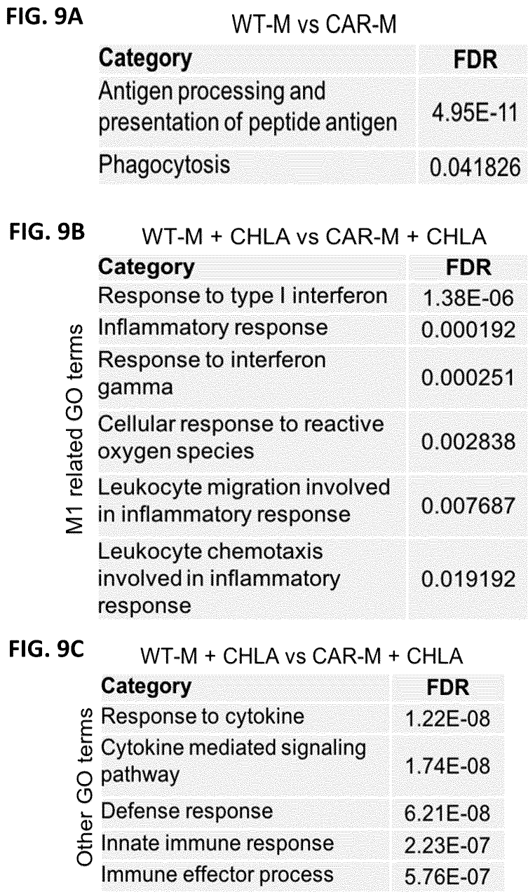

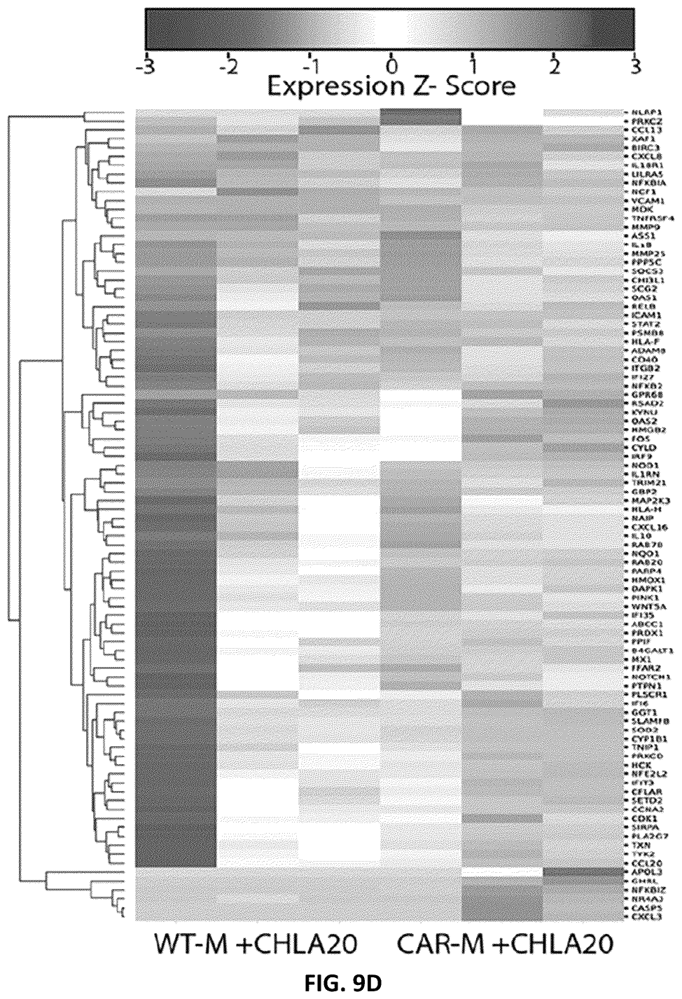

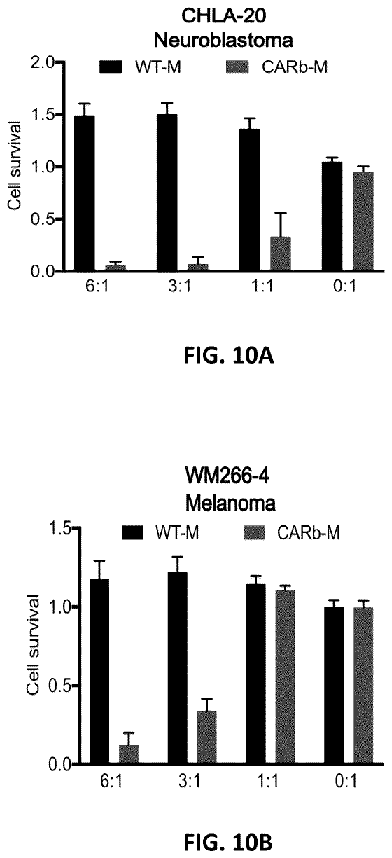

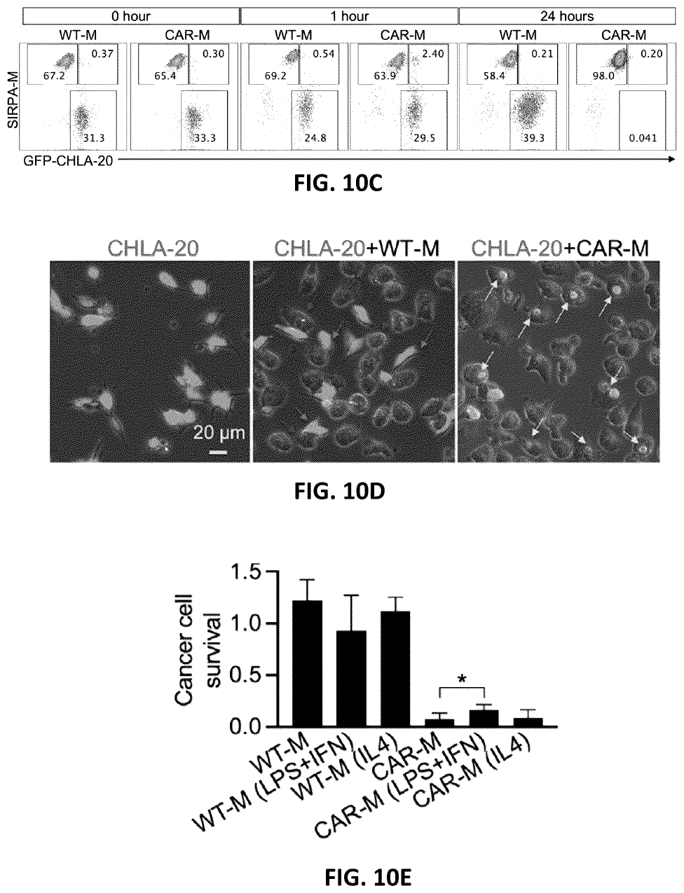

To further characterize the GD2-CARb macrophages, RNA-sequencing was performed. Gene ontology (GO) analysis, a method to interpret gene sets using GO ontology and annotation that group genes based on their function, revealed that M1 related pathways, including antigen processing and presentation of peptide antigen and phagocytosis, were enriched in GD2-CARb macrophages compared to WT-macrophages (FIG. 9A). After co-cultured with CHLA-20 cancer cells, more M1 related pathways were enriched in GD2-CARb macrophages, namely response to type I interferon, Inflammatory response, response to interferon gamma, cellular response to reactive oxygen species, leukocyte migration involved in inflammatory response, leukocyte chemotaxis involved in inflammatory response (FIG. 9B). Co-cultured with CHLA-20 cancer cells also increased immune response ability of GD2-CARb macrophages, as demonstrated by the enrichment of these GO term: response to cytokine, cytokine mediated signaling pathway, defense response, innate immune response, and immune effector process (FIG. 9C). All the results suggested that anti-cancer M1 like macrophages were generated from GD2-CAR engineered human pluripotent stem cells. Heatmap analysis also revealed the upregulation of numerous M1-related genes in CAR-Ms co-cultured with CHLA-20 (FIG. 9D). To test GD2-CAR-macrophage anti-tumor activity in vitro, GD2-CAR macrophages were exposed to positive CHLA-20-AkaLuc-eGFP (neuroblastoma) and WM266-4-AkaLuc-eGFP (melanoma) cells. GD2-CARb macrophage eliminated GD2 positive CHLA-20 and WM266-4 cells in vitro. CHLA-20 and WM266-4 cells were mono-cultured or co-cultured with different ratio of WT-macrophage or GD2-CARb macrophage for 20-24 hours. Luminescent assay was used to measure cell survival. Macrophage: cancer cell ratio was shown in 6:1, 3:1, 1:1 and 0:1 (FIG. 10A and FIG. 10B). To further explore the interaction between tumor cells (GFP labeled) and macrophages (SIRPa stained), we performed flow cytometric analysis of co-cultured cells at different time points (FIG. 10C). At 24 h, CHLA-20 cells were not reduced by WT-Ms, while most of the CHLA-20 cells were eliminated by CAR-Ms (FIG. 10C). The GFP⁺SIRPa⁺ cells were found at 1 h of CHLA-20 and CAR-M co-culture by flow cytometry (FIG. 10C), indicating that CHLA-20 cells were engulfed by CAR-Ms. Fluorescence imaging further confirmed phagocytosis of CHLA-20 cells by CAR-Ms (FIG. 10D, yellow arrows indicated).

To investigate whether the antitumor activity of CAR-Ms was dependent on the M1/M2 phenotype, we pre-treated WT-Ms and CAR-Ms with LPS and IFNγ or IL-4. Interestingly, although LPS and IFNγ were able to enhance M1 marker expression (FIG. 7C and FIG. 7D), they failed to improve the antitumor activity of both WT-Ms and CAR-Ms (FIG. 10E). Instead, the antitumor activity of CAR-Ms was slightly reduced (FIG. 10E). IL-4 promoted M2 polarization (FIG. 7C and FIG. 7D) but did not suppress the antitumor activity of CAR-Ms (FIG. 10E). The results suggested that the antitumor activity of CAR-Ms was independent of the M1/M2 phenotype induced by LPS, IFNγ, and IL-4.

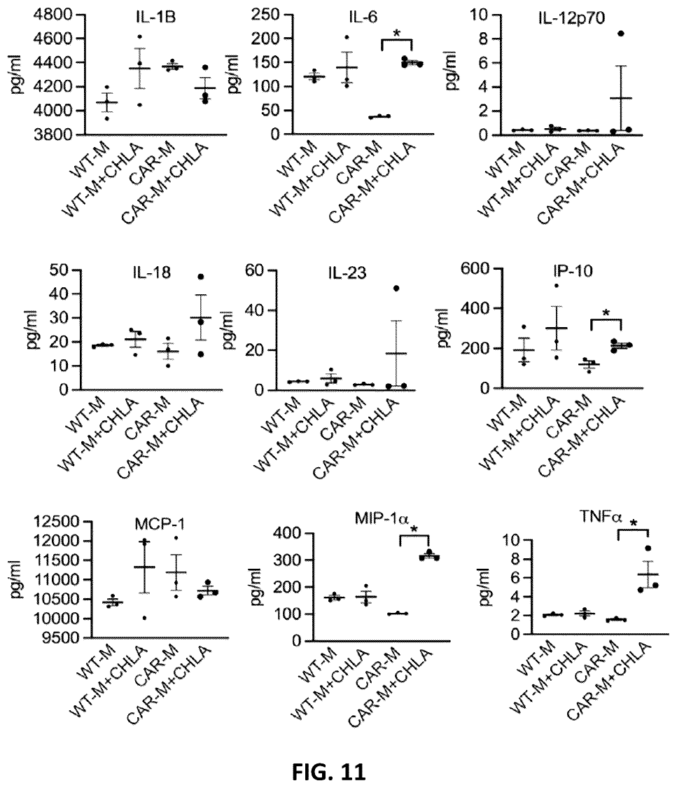

CAR-T cell therapy could trigger life-threatening cytokine-release syndrome (CRS), so we measured the cytokine release of macrophages before and after they were co-cultured with CHLA-20 cells. CRS-related cytokines can increase 30- to 8,000-fold upon CAR-T injection (Lee et al., 2019, Nat Commun. 10:2681; Norelli et al., 2018, Nat. Med. 24:739-748). In our results, IL-6, IP-10, MIP-1α, and tumor necrosis factor α (TNF-α) were only increased 2- to 4-fold in CAR-Ms after co-culture with CHLA-20 (FIG. 11), indicating minimal risk of CRS from CAR-Ms.

### Example 2: Anti-Tumor Activity of GD2-CAR Macrophages In Vivo

To verify antitumor activity in vivo, 5×10⁵ CHLA-20-AkaLuc-GFP cells were subcutaneously injected into the hind flank of NSG mice alone or with 2.5×10⁶ WT-M or CAR-M (H9 hPSC derived) (5:1 E:T ratio) (FIG. 12A). These studies revealed that CAR-M-treated mice had significantly reduced tumor burden 4 weeks post injection as evidenced by 90% tumor-free mice with CAR-M treatment compared with 50% tumor-free mice with WT-M treatment (FIG. 12D). Significant differences in tumor burden were observed between mice injected with CHLA-20 alone versus mice injected with both CHLA-20 and CAR-Ms, while no statistical differences in tumor burden were observed between mice injected with both CHLA-20 and WT-Ms versus mice with CHLA-20 alone (FIG. 12B). CAR-M treatment did not reduce the body weight (FIG. 12C), indicating minimal adverse effects of the cell therapy.

These results showed that hematopoietic progenitor/stem cells provided a promising source for anti-cancer immunotherapy. As set forth herein, a high efficient, fully defined, xeno-free method was established for deriving hematopoietic progenitor cells through arterial endothelial to hematopoietic transition that can be further differentiated into anti-tumor GD2-CAR macrophage in feeder-free and serum-free conditions. Compared to the previous study that generated 18-50-fold of macrophages from starting pluripotent stem cells (Zhang et al., 2020, J Hematol Oncol 13: 153), protocols using arterial endothelial to hematopoietic transition were able to generate about 300-fold of macrophages. Given that macrophages are able to penetrate into the solid tumor, human pluripotent stem cell derived-GD2-CAR macrophages provides a great alternative for CAR-T and CAR-NK in solid tumor immunotherapy.

### Example 3: CAR-M and CAR-EOS Enhance Anti-Tumor Activity of CAR-NK In Vitro

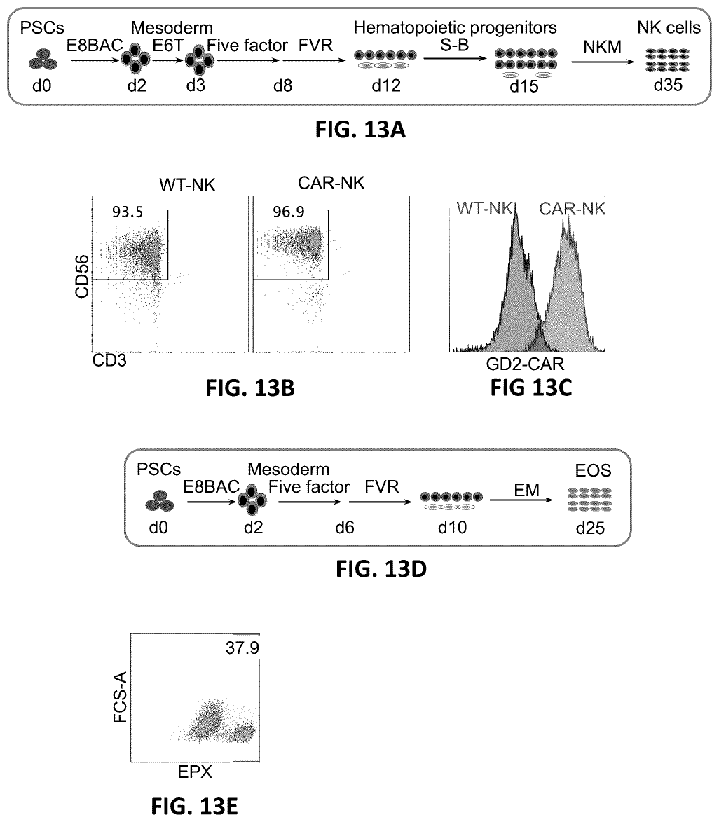

The interaction between white blood cells plays a key role in immune response. For example, macrophages can activate NK cells through direct cell-to-cell contact and through soluble cytokines such as IL-12, IL15, and IL-18. NK cells secrete IFNγ, which in turn activates macrophages. The positive feedback between macrophage and NK cells increases the activation of both types of cells. Combining CAR-M with CAR-NK, and CAR-EOS with CAR-NK can be more effective at killing tumor cells than each of these cell types alone. First, NK and EOS expressed GD2-CAR construct were generated. While these cell types can be transduced efficiently with adenoviral vectors, these vectors do not integrate into genomes and are unlikely to sustain prolonged CAR expression. Therefore, to generate CAR-NK and CAR-EOS, PSCs were first transduced with GD2 CARs integrated into AAVS2 locus and then differentiated into the respective hematopoietic cells (shown in FIG. 13A and FIG. 13D). The identity of NK, M and EOS derived from GD2-CAR PSCs was confirmed by high level expression of their respective biological marker proteins (CD3, CD5, CD11b, CD14, and EPX) measured by flow cytometry (shown in FIG. 13B and FIG. 13E). GD2-CAR expression was also confirmed in these cells as opposed to WT cells that derived from untransduced PSCs (shown in FIG. 13C).

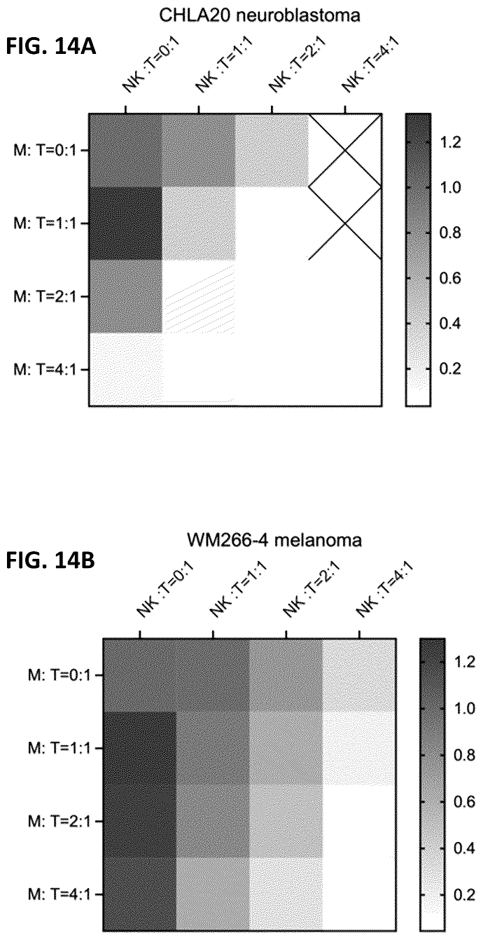

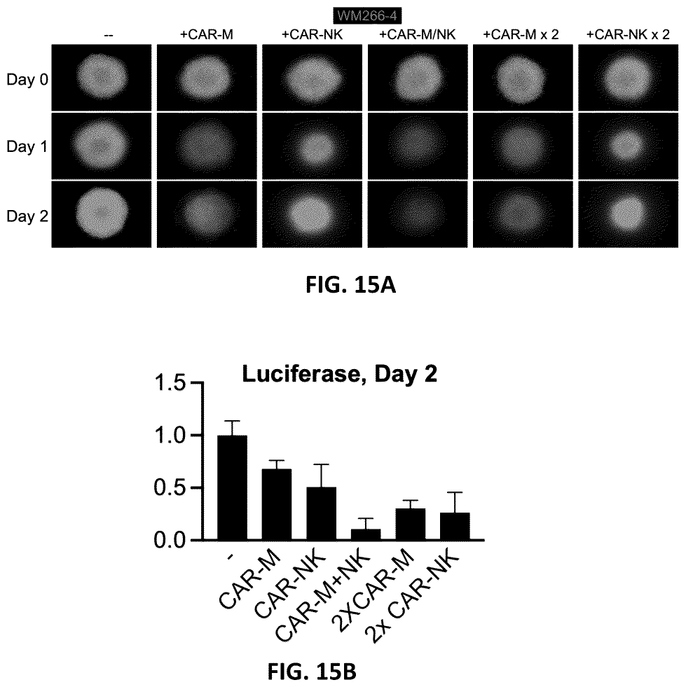

The antitumor activity of the combination of CAR-NK and CAR-M in vitro was evaluated, wherein CAR-NK cells demonstrated antitumor against CHLA-20 neuroblastoma cells at effector:target (E:T) ratio of 1:1. Antitumor activity was increased with higher E:T ratio (shown in FIG. 14A). Antitumor activity of CAR-NK cells was further improved when combined with CAR-M, wherein at NK:M:T ratios of 2:2:1 or higher, all CHLA-20 cells were killed. CAR-NK cells also displayed antitumor activity against WM266-4 melanoma cells at effector:target (E:T) ratio of 2:1 or higher (shown in FIG. 14B). Although CAR-M alone was not able to inhibit WM266-4 cells cultured with a mixed media of 50% DM5 and 50% NKM, combinations of CAR-M and CAR-NK demonstrated superior antitumor activity compared to either CAR-M or CAR-NK alone. AtNK:M:T ratios of 4:4:1, almost all WM266-4 cells were killed. Additional luciferase assays show that co-culture with both CAR-M and CAR-NK suppressed WM266-4 cell survival more efficiently than CAR-M or CAR-NK cells alone, even at higher doses (×2) of these cells (FIG. 15A and FIG. 15B).

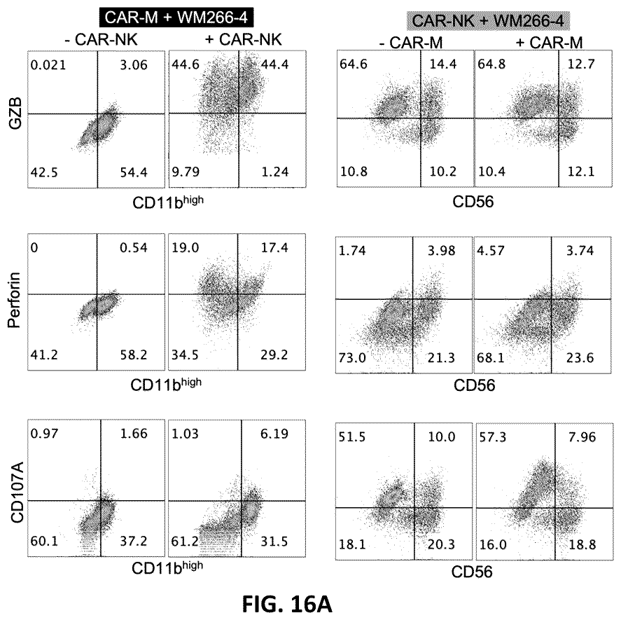

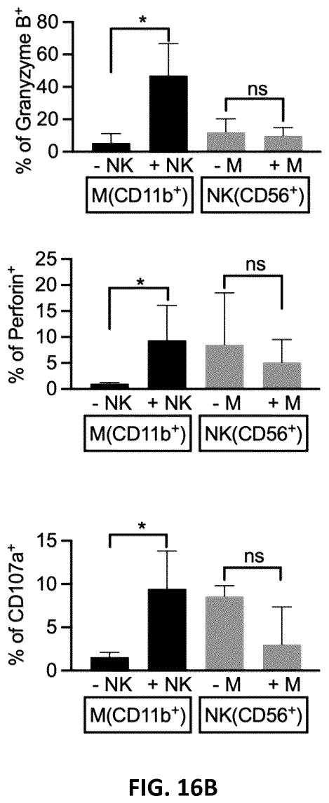

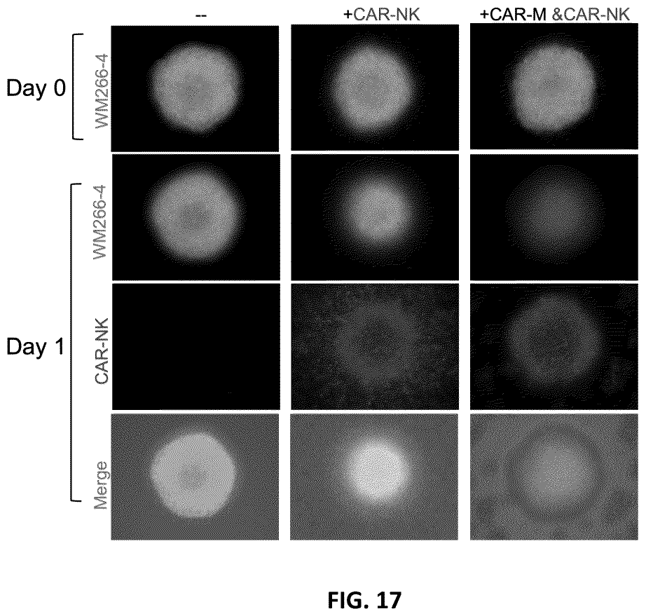

Granzyme B, perforin, and CD107a gene expressions (important for granule-mediated cell killing pathway) on CD11b CAR-M increased when co-culture with CD56 CAR-NK cells. However, expression of these genes remained unaltered in CD56 CAR-NK cells when co-culture with CAR-Ms (FIG. 16A and FIG. 16B). It was also observed that co-culturing CAR-NK with CAR-M caused more CAR-NK to attach to the melanoma cells than when only CAR-NK cells were added (FIG. 17).

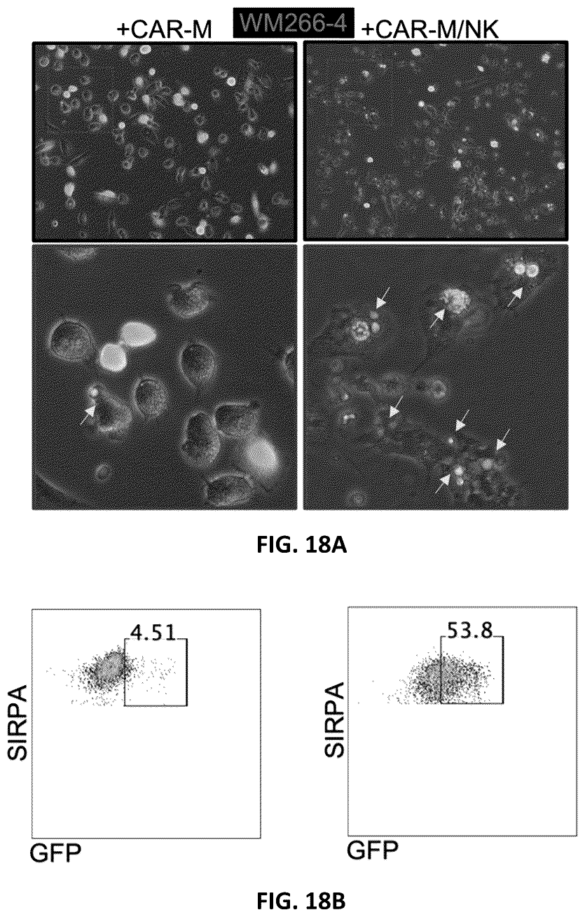

The phagocytosis activity CAR-M (engulfment of tumor cells by CAR-M) was increased in the presence of CAR-NK (FIG. 18A and FIG. 18B). Yellow arrows indicated tumor cells (green cells) that were engulfed by CAR-M (FIG. 18A). The phagocytosis activity CAR-M was further confirmed by flow cytometric analysis (FIG. 18B). WM266-4 was labeled with GFP and CAR-M was labeled by SIRPa. The GFP⁺SIRPa⁺ cells indicated phagocytosis of WM266-4 cells by CAR-M.

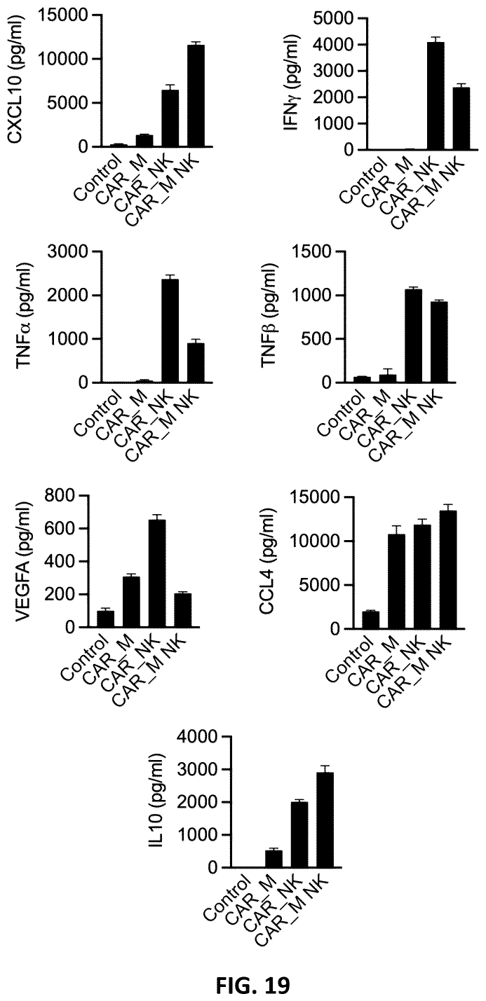

Secretome analysis revealed that expression of cytokines commonly found in tumor microenvironments was altered. For example, CXCL10 and IL10 significantly increased, while IFNγ, TNFα, and VEGFA decreased in media with both CAR-M and CAR-NK compared to each CAR alone when co-culturing with WM266-4 cells (FIG. 19).

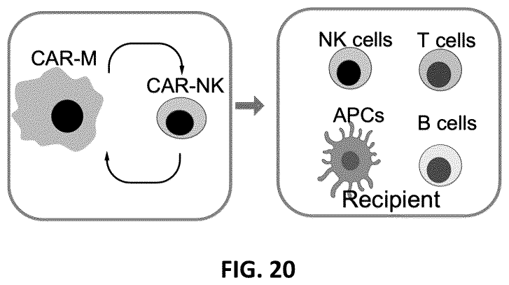

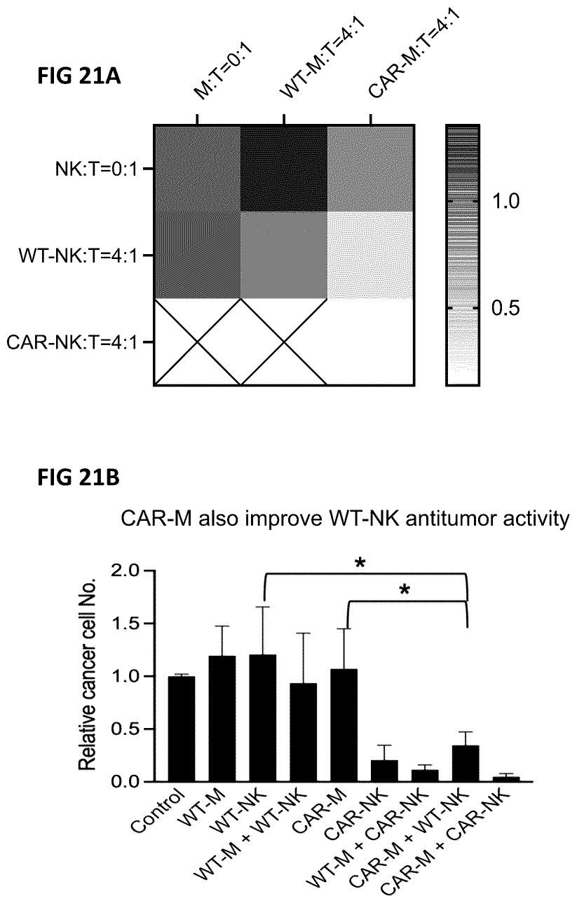

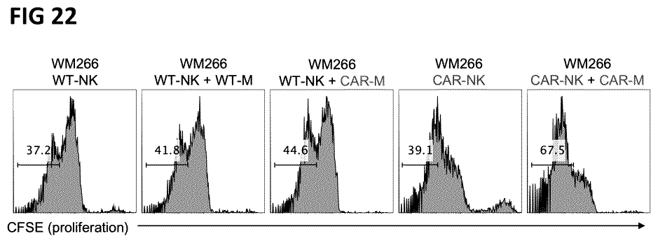

Combinations of WT-M and WT-NK, CAR-M and WT-NK also demonstrated increased antitumor activity indicating that CAR-M might be able to activate recipient's immune cells such as recipient native NK cells (shown in FIG. 20, FIG. 21A and FIG. 21B). Further analysis revealed that both WT-M and CAR-M promoted NK cell proliferation in this of tumor cells (shown in FIG. 22). WT-M increased proliferation of WT-NK from 37% to 42%, while CAR-M increased it to 45%. CAR-M had more significant effects on CAR-NK proliferation, as demonstrated by increasing proliferation from 39% to 68%. These proliferation results were highly correlated with antitumor activity shown in FIG. 21A and FIG. 21B.

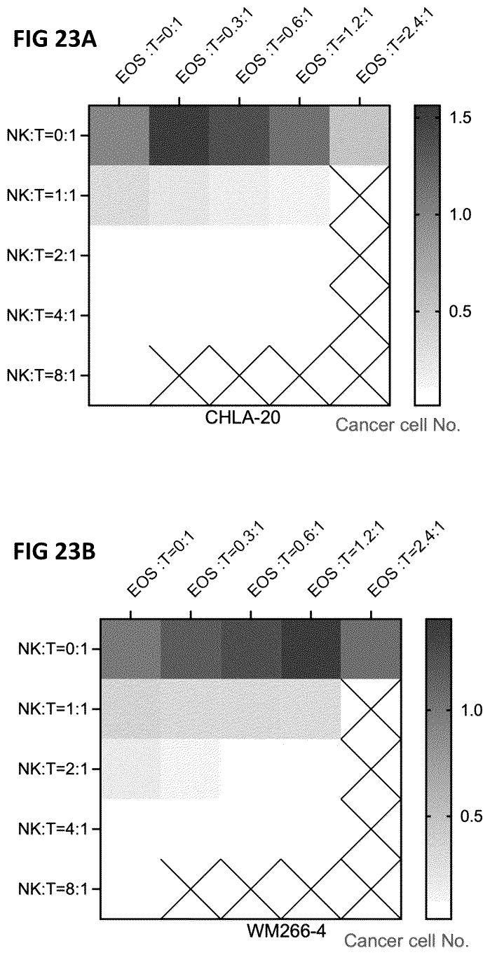

The antitumor activity of the combination of CAR-NK and CAR-EOS in vitro was also investigated (shown in FIG. 23A and FIG. 23B). These results demonstrated that CAR-EOS alone had no antitumor activity at 0.3:1 to 1.2:1 E:T ratios, but improved CAR-NK antitumor activity for CHLA20 cells at EOS:NK:T ratios of 0.6:1:1 and 1.2:1:1 (shown in FIG. 23A). CAR-EOS also improved CAR-NK antitumor activity for WM266-4 at NK:T ratios of 2:1 ratio (shown in FIG. 23B).

### Example 4: CAR-M and CAR-EOS Enhance Anti-Tumor Activity of CAR-NK In Vivo

To determine the anti-tumor activity of CAR-M and CAR-EOS in combination with CAR-NK in vivo, a cytotoxicity assay with melanoma tumor cells in a mice xenograft model will be used.

Mice were injected on the hind flank with 2×10⁵ AkaLuc-GFP-expressing WM-266-4 melanoma cells. Three days later, melanoma cell engraftment was assessed by IVIS imaging for baseline pretreatment reading, and 2×10⁶ CAR-M and/or CAR-NK were administered to the mice by intravenous injection. For CAR-NK×2, 4×10⁶ CAR-NK were administered by intravenous injection. Tumor burden was determined by bioluminescent imaging at 0-, 7-, and 14-days post CAR-MINK injection. GD2 CAR-M and CAR-NK combination substantially reduced the tumor burden as compared to CAR-M or CAR-NK alone, and to mice injected with melanoma cells by 14 days (FIG. 24B).

### Example 5: Characterizing Anti-Tumor Activity of GD2-CAR Neutrophils In Vitro

To determine whether immunotherapy with neutrophils engineered to express a GD2-specific chimeric antigen receptor (GD2-CAR neutrophils) will demonstrate potent anti-tumor activity, CAR-GD2 cell lines were used to generate neutrophils in serum-free, xeno-free, and feeder-free conditions using ETV2 modified mRNA-based differentiation system. Additionally, to characterize CAR-neutrophils, structural and functional aspects of the cells were evaluated.

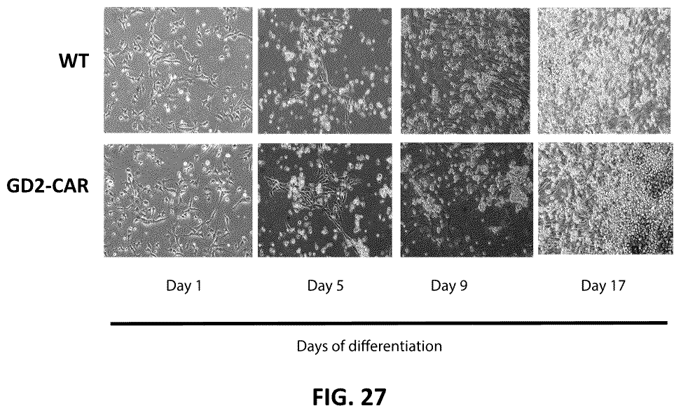

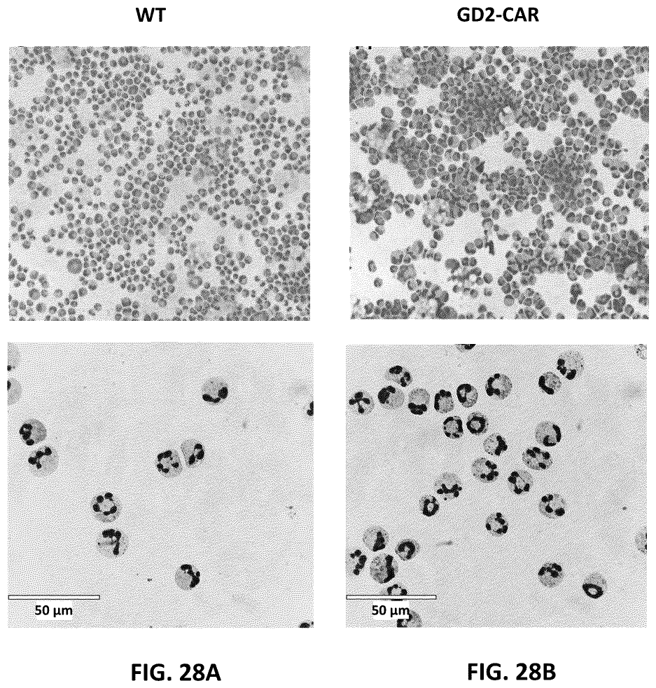

Morphology. Transfection of ETV2 mmRNA into the single cell of wild-type and CAR-GD2 hPSCs form a typical endothelial morphology within 24 hours (FIG. 27). During the process of differentiation, cell changes it morphology and form floating myeloid progenitors (FIG. 27). After terminal differentiation, neutrophil can be identified by is Wright-Giemsa staining (FIG. 28A and FIG. 28B).

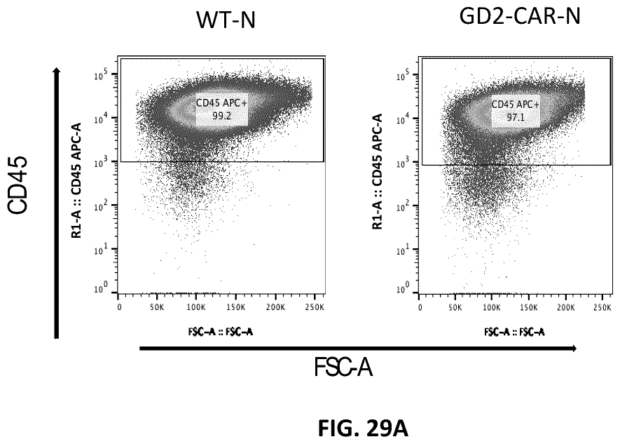

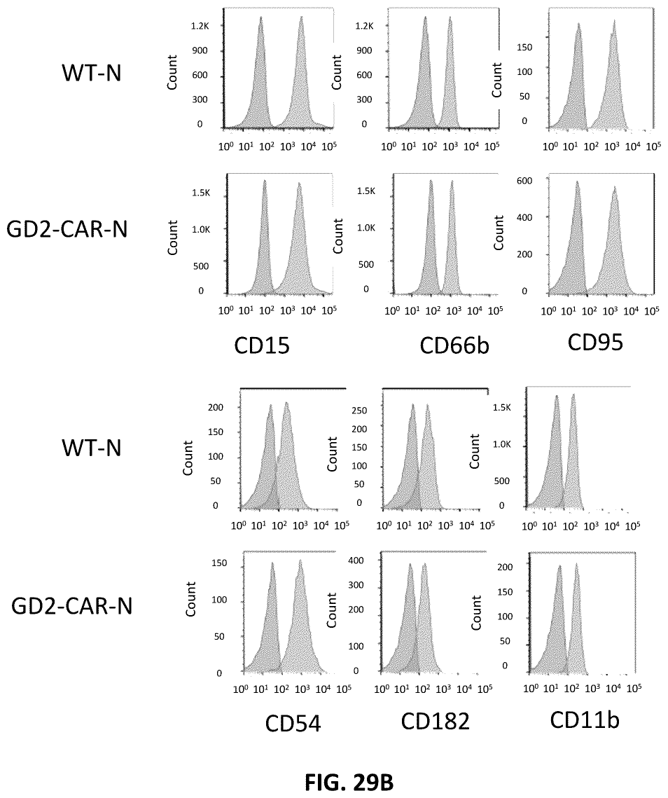

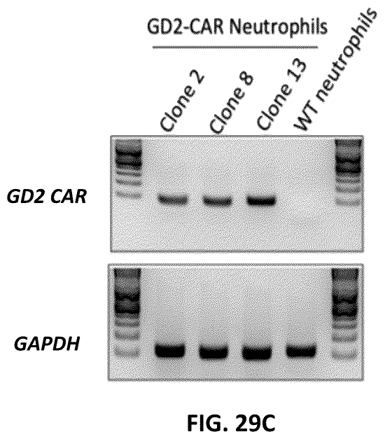

Cell surface markers. CD11b, CD16, CD15, CD66b, CD95, CD54 and CD182 expression in terminal differentiated WT and GD2-CAR neutrophils proves ETV2 mmRNA induced granulocytic differentiation program (FIG. 29A through FIG. 29G).

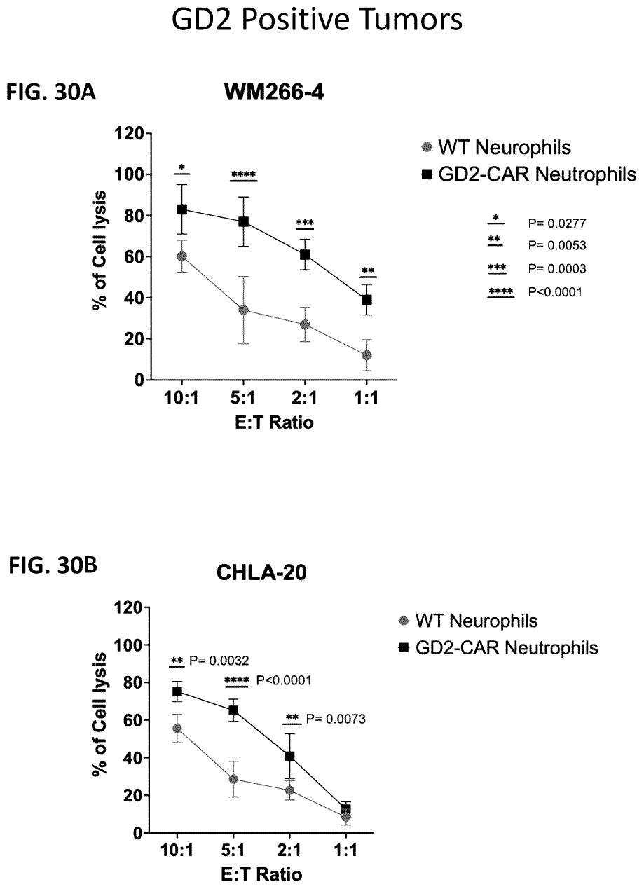

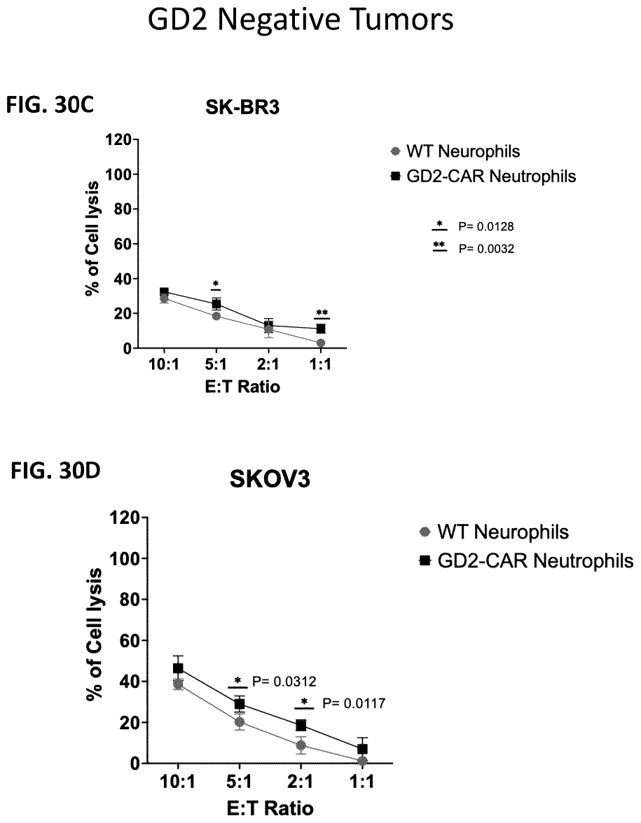

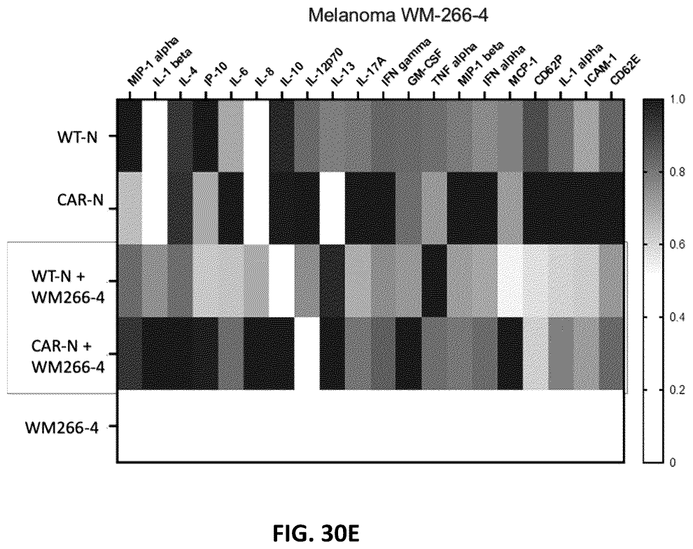

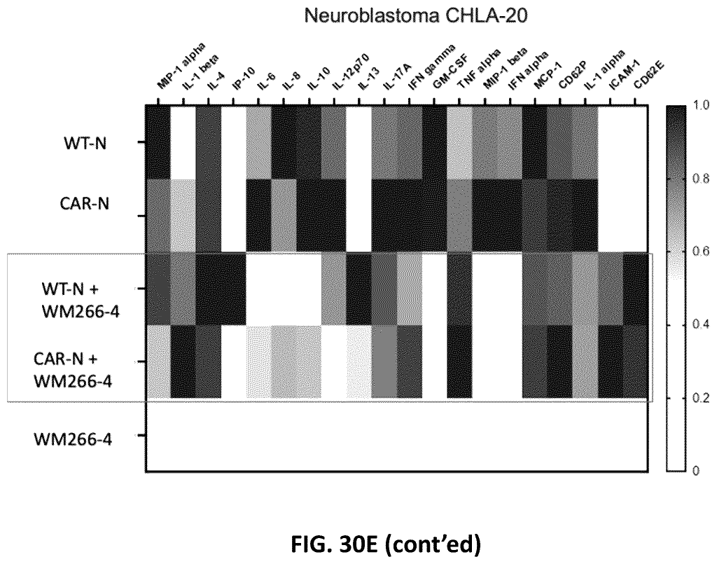

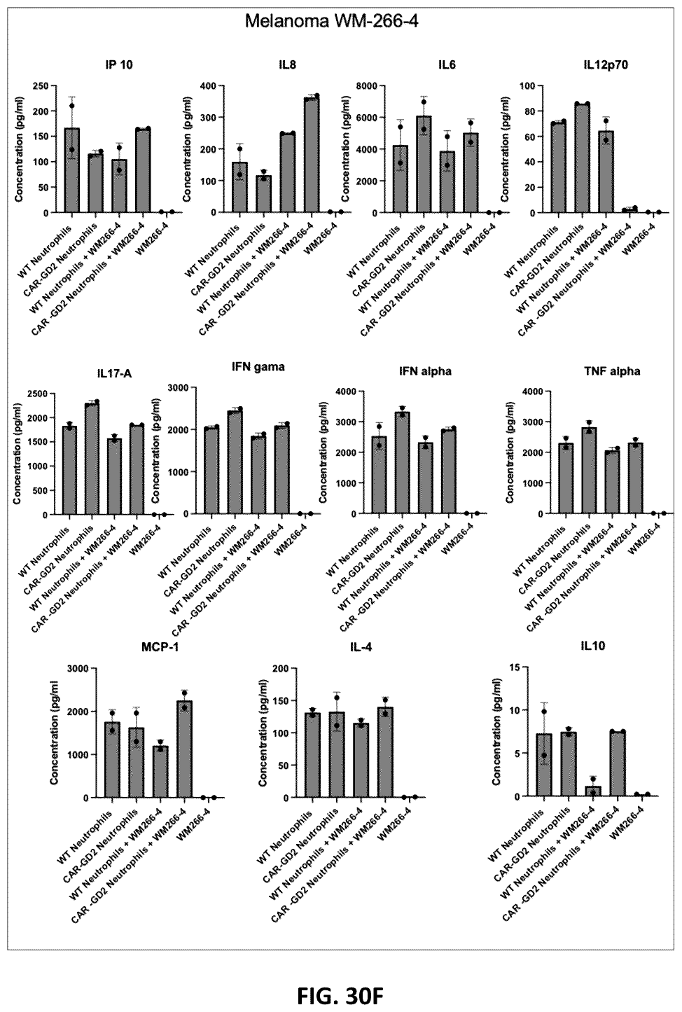

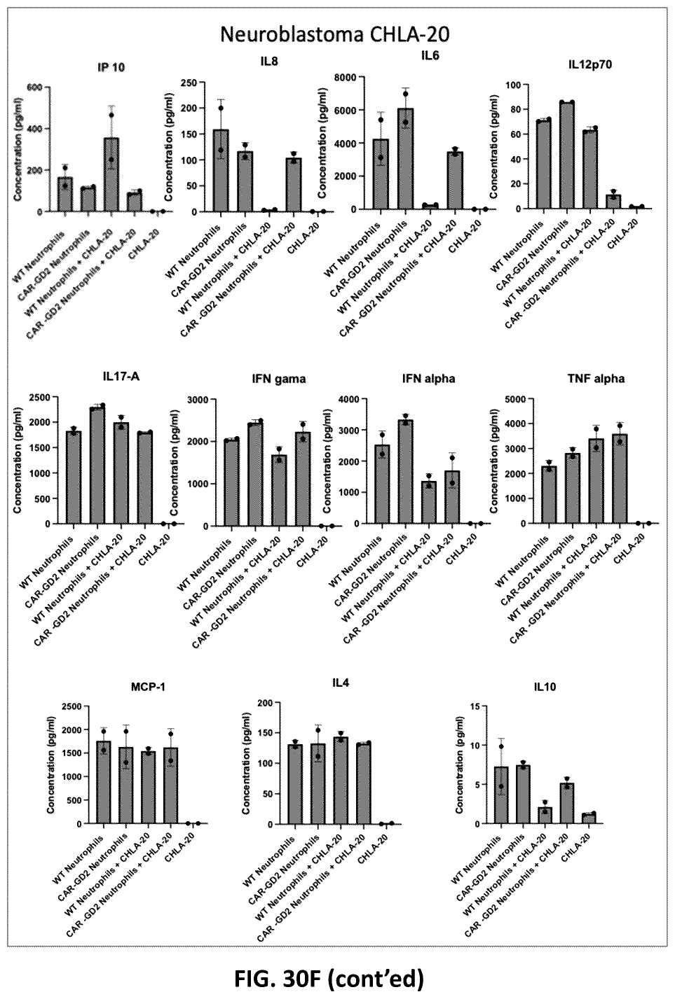

Functional evaluations. Functional characterization of ETV2 mmRNA induced neutrophils can be determined by cytotoxicity assay (FIG. 30A through FIG. 30D). As compared to wild-type neutrophils, CAR-GD2 hPSCs neutrophils demonstrated higher cytotoxicity against GD2 expressing tumors melanoma, and neuroblastoma (FIG. 30A and FIG. 30B). No differences in cytotoxicity of wild-type and CAR-GD2 neutrophils against GD2-negative tumors SKOV3 (ovarian cancer cells) and SK-BR3 (human breast cancer cells) was observed (FIG. 30C and FIG. 30D). GD2-CAR Neutrophils secreted inflammatory cytokines after co-culture with GD2 positive tumor cells (FIG. 30E and FIG. 30F). Overall, this invention provided a method for generating CAR-GD2 hPSCs neutrophils with superior anti-tumor activity for therapeutic purposes.

Quantification. ETV2 mmRNA induction produced 1.7×10⁶ neutrophils from 10⁶ wild-type hPSCs and 2×10⁶ neutrophils from 10⁶ CAR-GD2 hPSCs within 3 weeks.

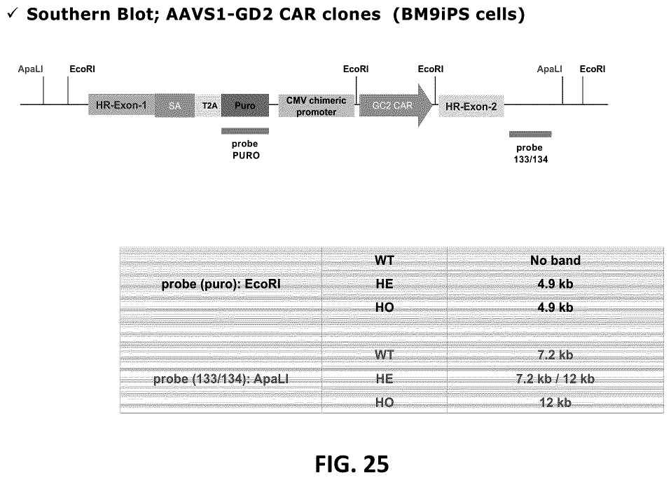

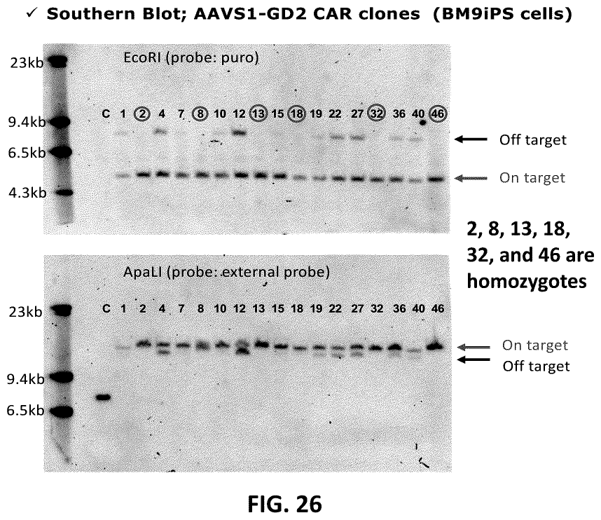

Southern Blot. Human iPSCs were transfected with sgRNA, CAS9 protein with GD2-CAR vector and selected by puromycin. Puromycin-resistant cells were clonally expanded and on-targeted clones were selected by southern blot. Southern blot analysis was performed by DIG-labeling hybridization. The external probe is a DIG-labeled fragment that binds to the ApaLI digested fragment of 5′ external region. The internal probe is a DIG-labeled fragment that binds to the EcoRI digested fragment of the puromycin region. Southern blot analysis confirmed that GD2-CAR clones established from BM9 iPSC line had the GD2-CAR construct inserted into the correct position at AAVS1 locus (FIG. 25 and FIG. 26).

As illustrated by these results, PSC-based technologies for neutrophil and macrophage manufacturing offer opportunity to generate cellular product with uniform biological features that can be produced in nearly infinite amounts and therefore providing a straightforward pathway for commercialization. Characterization of the resulting CAR-GD2 neutrophils (made either from ESCs or from iPSCs) confirmed CD11b and CD16 expression, and the CAR-neutrophils demonstrated superior anti-tumor phagocytic activity against tumor cells, relative to wild-type neutrophils. These methods allow for scalable production of clinical grade CAR-GD2 neutrophils from human hPSCs for off-the-shelf immunotherapies. Since neutrophils are short-lived and their genetic modification is longstanding challenge, this hPSC-based technology provides a superior opportunity to generate genetically-modified neutrophils for clinical applications

iPSC-based technologies for neutrophil manufacturing offer opportunity to generate cellular products with uniform biological features that can be produced in nearly infinite amounts and therefore provide a straightforward pathway for commercialization. This disclosure provides methods for producing CAR-GD2 neutrophils that, alone or in combination with other cell products (such as the CAR-GD2 macrophages), could help target GD2-expressing tumors for destruction.

### Example 6: Anti-Tumor Activity of GD2-CAR Neutrophils In Vivo

To determine the anti-tumor activity of GD2-CAR neutrophils in vivo, a cytotoxicity assay with melanoma tumor cells in a mice xenograft model was used.

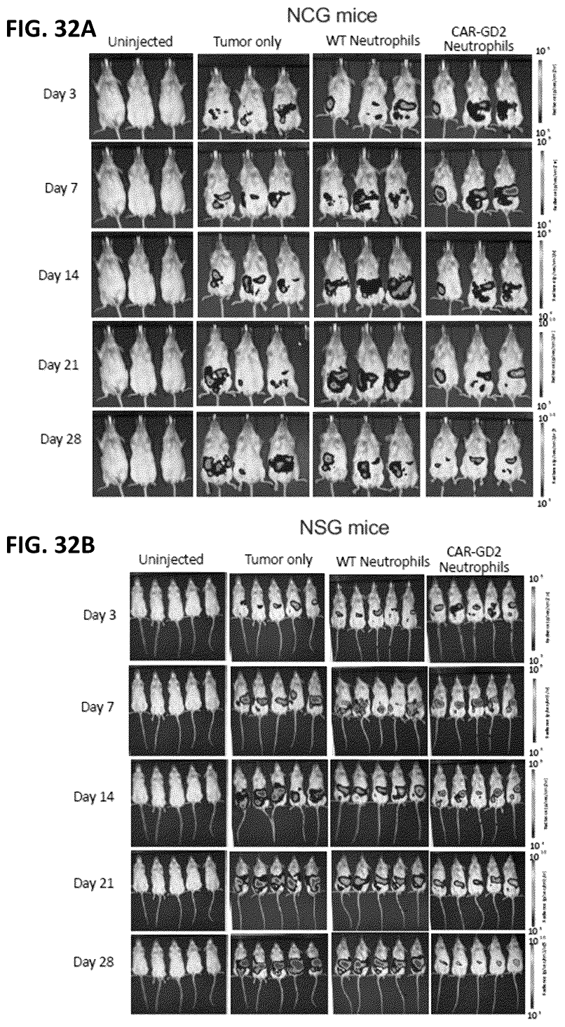

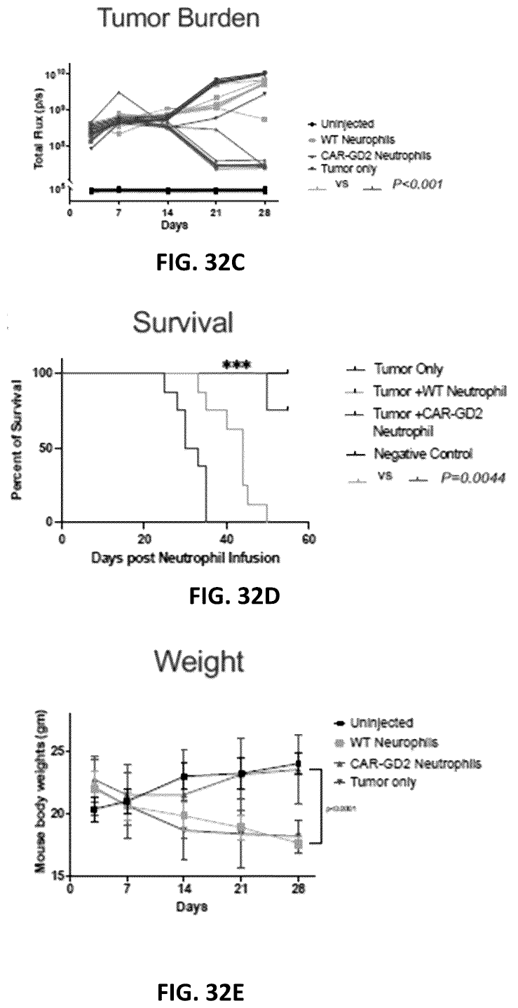

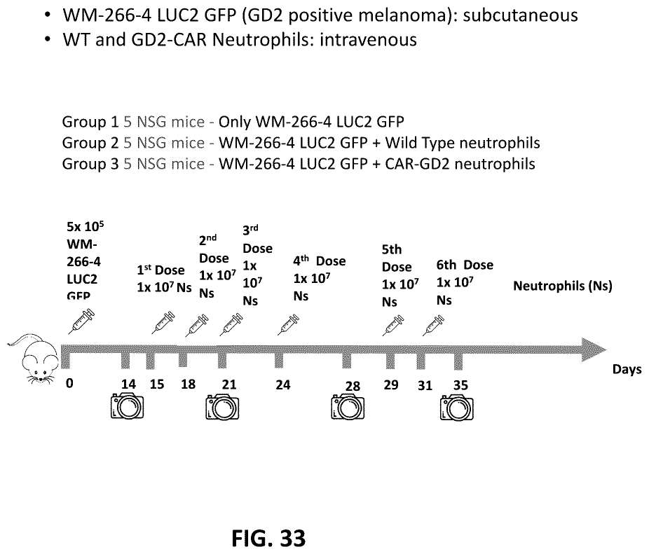

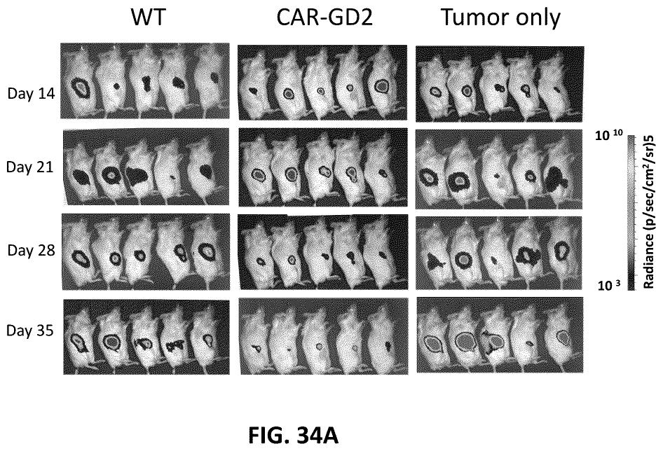

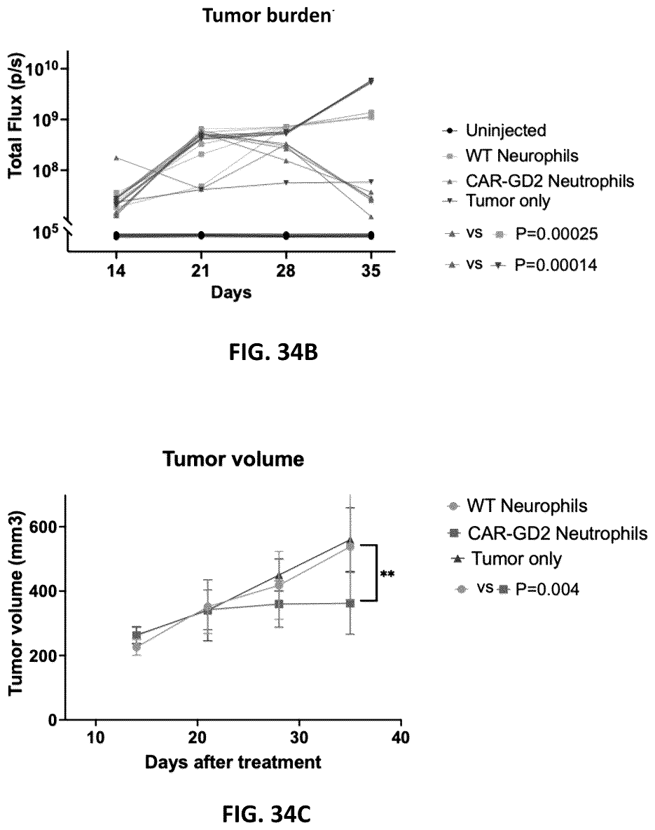

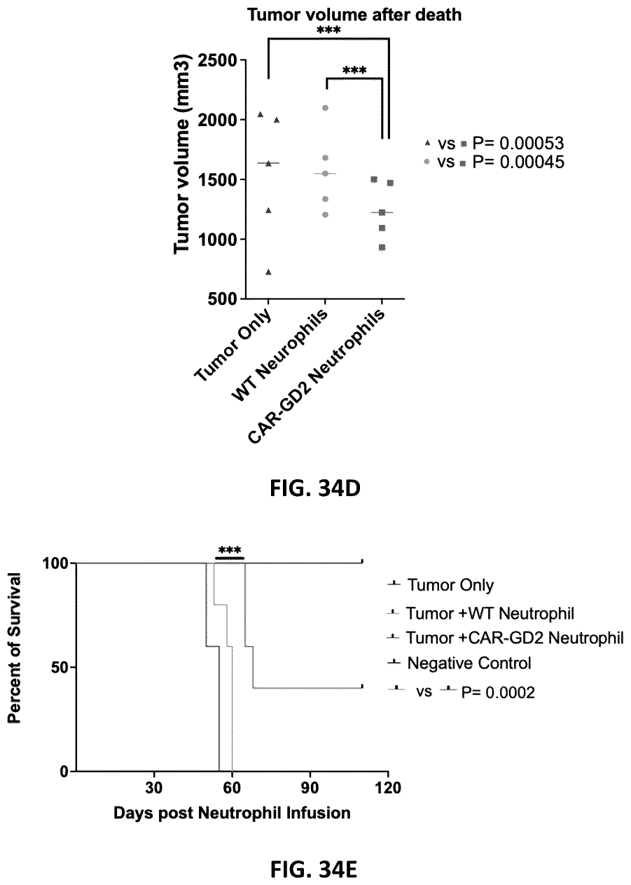

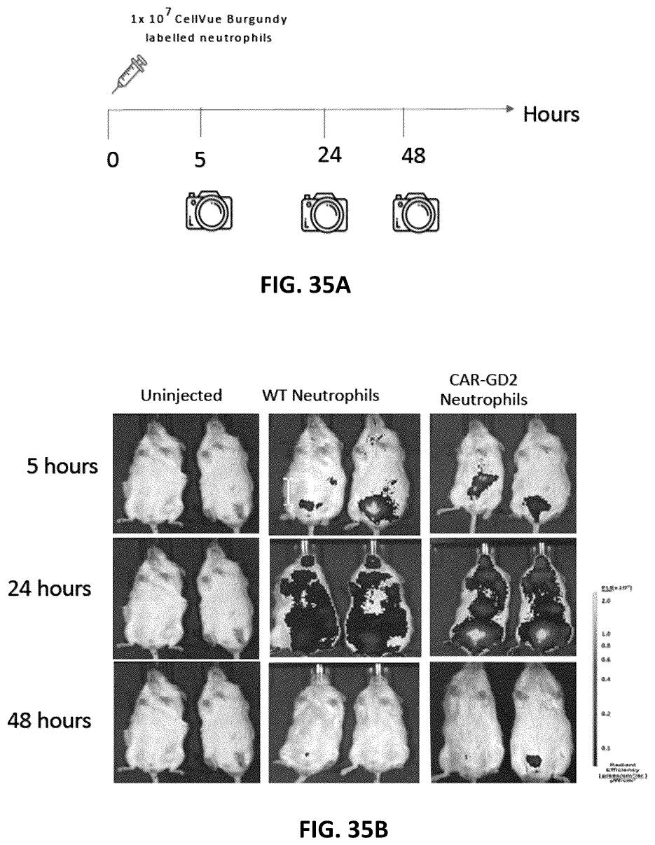

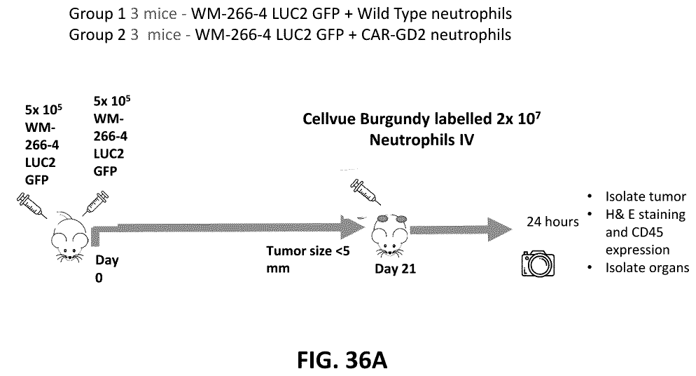

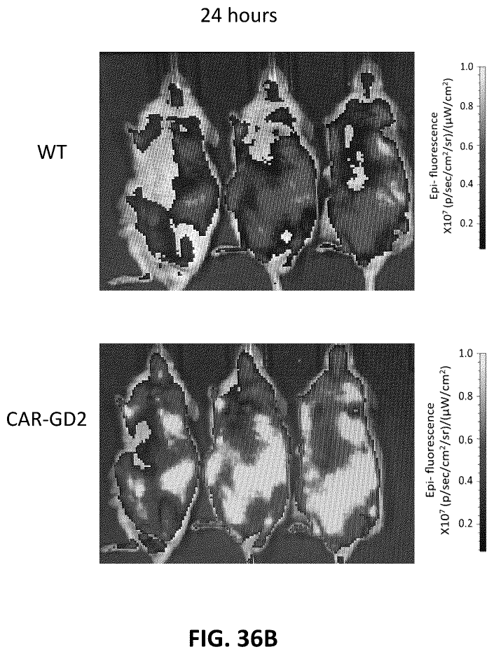

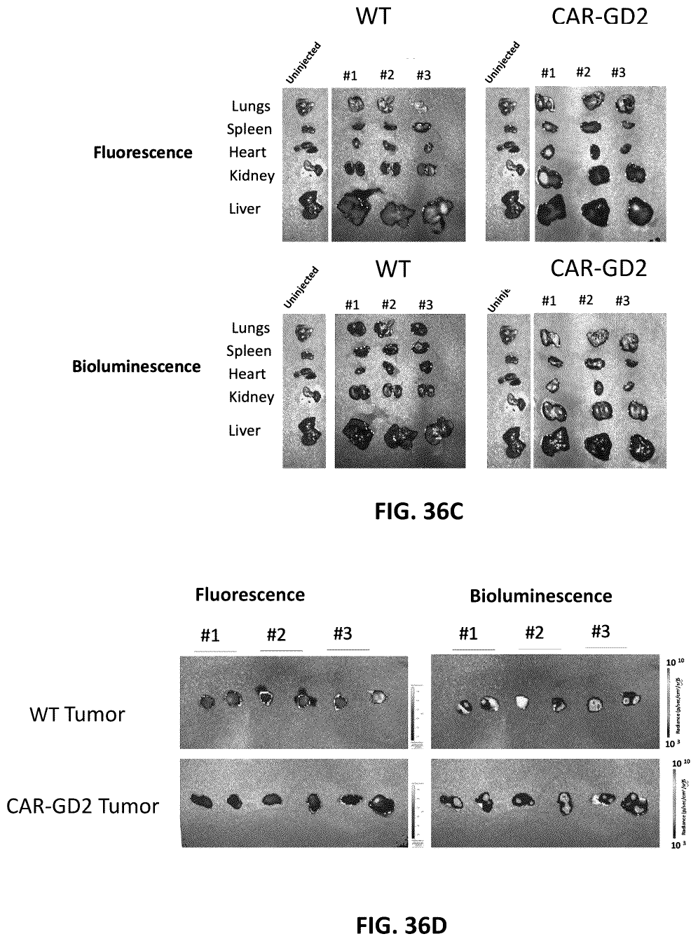

Mice were inoculated intraperitoneally with 3×10⁵ Luc2-eGFP-expressing WM-266-4 melanoma cells. Melanoma cell engraftment was assessed by IVIS imaging 3 days later for baseline pretreatment reading. On day 4 after melanoma injection, mice were left untreated, or treated with 107 unmodified neutrophils or GD2-CAR-neutrophils injected intraperitoneally every 7 days. Tumor burden was determined by bioluminescent imaging. A schematic of the cytotoxicity model is shown in FIG. 31. Group 1 (2 male and 1 female mice) were injected with WM-266-4 LUC2 GFP cells only; Group 2 (2 male and 1 female mice) were injected with WM-266-4-LUC2-eGFP cells and wild-type neutrophils; Group 3 (2 male and 1 female mice) were injected with WM-266-4-LUC2-eGFP cells and GD2-CAR neutrophils; and Group 4 (2 male and 1 female mice) were un-injected. GD2-CAR neutrophils substantially reduced the tumor burden in the ventral axis and dorsal axis and as compared to WT neutrophils and mice injected with melanoma cells by 28 days (FIG. 32C through 32E). Similar results were also obtained in a clinically relevant mouse model in which 5×10⁵ Luc2-eGFP-expressing WM-266-4 melanoma cells were injected subcutaneously (FIG. 33). FIG. 34A through FIG. 34E) show that tumor volume was decreased and survival rate was increased in mice receiving anti-GD2 CAR-N compared to WT neutrophils and control. Furthermore, GD2-CAR Neutrophils were distributed in the body in a time-dependent manner (FIG. 35A and FIG. 35B). In addition, GD2-CAR neutrophils were distributed in other organs (shown by the fluorescence) with metastatic tumor (shown by bioluminescence) (FIG. 36C). GD2-CAR and WT neutrophils were also detected in the subcutaneous tumor (FIG. 36C and FIG. 36D).

### Example 7: Knockout of Exon 3 Enhances the Antitumor Activity of CAR-Macrophages, but Knockout of Exon 2 Inhibits the Antitumor Activity

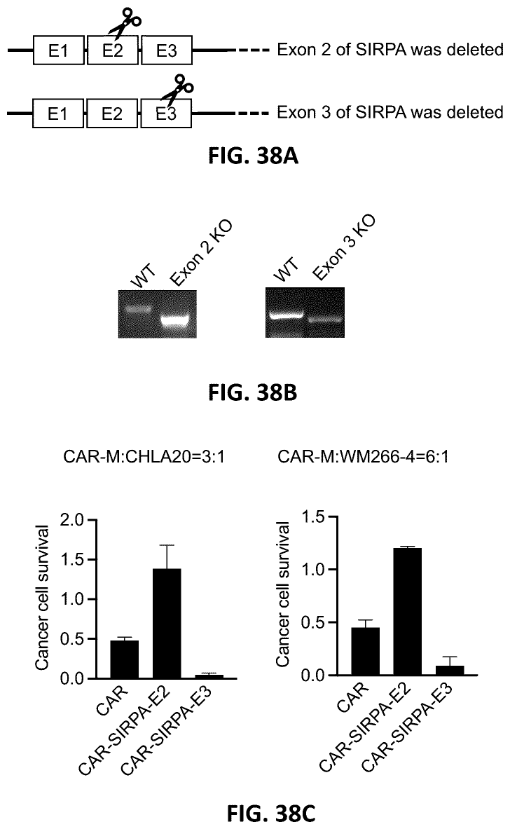

Many solid tumors have increased expression of CD47 receptors, one type of the protective (“don't eat me”) receptors, which recognize signal regulatory protein alpha (SIRPa) receptor on macrophages. Activation of CD47/SIRPa specifically blocked phagocytosis when activating phagocytic stimuli was present. Thus, targeting this pathway by knocking out SIRPa in macrophage had a potential anti-tumor activity. Indeed, CAR-M with knock-out SIRPa by exon 3 deletion (FIG. 37, FIG. 38A and FIG. 38B) showed improved killing of CHLA-20 neuroblastoma and WM266-4 melanoma (FIG. 38C). In contrast, deletion of exon 2 inhibited the tumor killing activity of CAR-M, suggesting that exon 3 deletion was specific to SIRPa's enhanced anti-tumor activity.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

1. A genetically engineered CD11b+ CD14+ macrophage, wherein the CD11b+ CD14+ macrophage expresses an anti-disialoganglioside GD2 (GD2) chimeric antigen receptor (CAR).

2. The genetically engineered CD11b+ CD14+ macrophage of claim 1, wherein the macrophage is produced from a pluripotent stem cell genetically engineered to express an anti-GD2 CAR.

3. The genetically engineered CD11b+ CD14+ macrophage of claim 2, wherein the pluripotent stem cell is an embryonic stem cell or an induced pluripotent stem cell.

4. The genetically engineered CD11b+ CD14+ macrophage of claim 1, wherein the macrophage express higher levels of CD80 and lower levels of CD163 and CD206 than a macrophage that does not express the anti-GD2 CAR.

5. The genetically engineered CD11b+ CD14+ macrophage of claim 1, wherein the macrophage is capable of inhibiting tumor cell proliferation and survival, and wherein the tumor cell expresses GD2.

6. The genetically engineered CD11b+ CD14+ macrophage of claim 1, wherein the macrophage selectively targets cells that express GD2 antigen.

7. The genetically engineered CD11b+ CD14+ macrophage of claim 5, wherein the tumor cell is a solid tumor.

8. The genetically engineered CD11b+ CD14+ macrophage of claim 5, wherein the tumor cell is not a blood cancer.

9. The genetically engineered CD11b+ CD14+ macrophage of claim 5, wherein the tumor cell is a neuroblastoma, retinoblastoma, medulloblastoma, glioblastoma, melanoma, lung cancer, pancreatic cancer, bladder cancer, colorectal cancer, sarcoma, or breast cancer cell.

10. The genetically engineered CD11b+ CD14+ macrophage of claim 1, wherein the anti-GD2 CAR has an amino acid sequence comprising SEQ ID NO:6 or a sequence having at least 80% sequence identity to SEQ ID NO:6.

11. The genetically engineered CD11b+ CD14+ macrophage of claim 1, wherein the anti-GD2 CAR comprises a nucleic acid sequence encoding an anti-GD2-scFv1 polypeptide, a hinge polypeptide, a CD28 transmembrane polypeptide, an OX40 polypeptide, and a CD3-zeta polypeptide.

12. The genetically engineered CD11b+ CD14+ macrophage of claim 1, wherein the macrophage exhibits an M1-like anti-cancer phenotype.

13. An isolated population of the genetically engineered CD11b+ CD14+ macrophages of claim 1.

14. The isolated population of claim 13, wherein the population comprises about 90% to about 99% CD11b+ CD14+ macrophages.

15. A method for producing the genetically engineered CD11b+ CD14+ macrophage of claim 1, the method comprising:
a) genetically engineering a pluripotent stem cell to express an anti-GD2 chimeric antigen receptor (CAR);
b) culturing the pluripotent stem cell in a first chemically defined medium for a sufficient time to produce a mesoderm cell;
c) culturing the mesoderm cell seeded at low density in a second chemically defined culture medium that comprises a fibroblast growth factor (FGF) and a vascular endothelial growth factor (VEGF) for a sufficient time to produce a hemogenic endothelial cell;
d) culturing the hemogenic endothelium cell in a third chemically defined culture medium for a sufficient time to produce a CD34+ CD45+ hematopoietic progenitor cells; and
e) culturing the CD34+ CD45+ hematopoietic progenitor cell in a fourth chemically defined culture medium for a sufficient time to produce a CD11b+ CD14+ macrophage.

16. The method of claim 15, wherein the pluripotent stem cell is an embryonic stem cell or an induced pluripotent stem cell.

17. The method of claim 15, wherein the genetically engineered CD11b+ CD14+ macrophage express higher levels of CD80 and lower levels of CD163 and CD206 than a macrophage that does not express the anti-GD2 CAR.

18. A method for producing the genetically engineered CD11b+ CD14+ macrophage of claim 1, the method comprising:
a) genetically engineering an hematopoietic progenitor cell (HPC) express an anti-GD2 chimeric antigen receptor (CAR), wherein the HPC was produced from pluripotent stem cells through arterialized hemogenic endothelium in a low-density culture; and
b) culturing the hematopoietic progenitor cell in a feeder-free and serum-free medium for a sufficient time to produce the CD11b+ CD14+ macrophage.

19. An isolated population of genetically engineered CD11b+ CD14+ macrophages obtained according to the method of claim 15.

20. An isolated population of genetically engineered CD11b+ CD14+ macrophages obtained according to the method of claim 18.

21. A pharmaceutical composition comprising the genetically engineered CD11b+ CD14+ macrophage of claim 1.

22. A method of treating a solid tumor in a subject in need thereof, the method comprising administering a therapeutically effective amount of the pharmaceutical composition of claim 21, and wherein the solid tumor expresses GD2.

23. A method of reducing proliferation or survival of a solid tumor cell, the method comprising contacting the solid tumor with the genetically engineered CD11b+ CD14+ macrophage of claim 1, and wherein the solid tumor expresses GD2.

24. The method of claim 22, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

25. The method of claim 23, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

26. A genetically engineered CD3− CD56+ natural killer cell, wherein the cell expresses an anti-disialoganglioside GD2 (GD2) chimeric antigen receptor (CAR) and is capable of inhibiting tumor cell proliferation or survival of cells expressing GD2 antigen, and wherein the tumor cell expresses GD2.

27. The genetically engineered CD3− CD56+ natural killer cell of claim 26, wherein the natural killer cell is produced from a pluripotent stem cell genetically engineered to express an anti-GD2 CAR.

28. The genetically engineered CD3− CD56+ natural killer cell of claim 27, wherein the pluripotent stem cell is an embryonic stem cell or an induced pluripotent stem cell.

29. The genetically engineered CD3− CD56+ natural killer cell of claim 26, wherein the natural killer cell selectively targets cells that express GD2 antigen.

30. The genetically engineered CD3− CD56+ natural killer cell of claim 26, wherein the tumor cell is a solid tumor.

31. The genetically engineered CD3− CD56+ natural killer cell of claim 26, wherein the tumor cell is not a blood cancer.

32. The genetically engineered CD3− CD56+ natural killer cell of claim 26, wherein the tumor cell is a neuroblastoma, retinoblastoma, medulloblastoma, glioblastoma, melanoma, lung cancer, pancreatic cancer, bladder cancer, colorectal cancer, sarcoma, or breast cancer cell.

33. The genetically engineered CD3− CD56+ natural killer cell of claim 26, wherein the anti-GD2 CAR has an amino acid sequence comprising SEQ ID NO:6, or a sequence having at least 80% sequence identity to SEQ ID NO:6.

34. The genetically engineered CD3− CD56+ natural killer cell of claim 26, wherein the anti-GD2 CAR comprises a nucleic acid sequence encoding an anti-GD2-scFv1 polypeptide, a hinge polypeptide, a CD28 transmembrane polypeptide, an OX40 polypeptide, and a CD3-zeta polypeptide.

35. An isolated population of the genetically engineered CD3− CD56+ natural killer cells of claim 26.

36. The isolated population of claim 35, wherein the population comprises about 90% to about 99% CD3− CD56+ natural killer cells.

37. A method for producing the genetically engineered CD3− CD56+ natural killer cell of claim 26, the method comprising:
a. genetically engineering a pluripotent stem cell to express an anti-GD2 CAR;
b. culturing the pluripotent stem cell in a first chemically defined medium for a sufficient time to produce a mesoderm cell;
c. culturing the mesoderm cell seeded at low density in a second chemically defined culture medium that comprises a fibroblast growth factor (FGF) and a vascular endothelial growth factor (VEGF) for a sufficient time to produce a hemogenic endothelial cell;
d. culturing the hemogenic endothelium cell in a third chemically defined culture medium for a sufficient time to produce a CD34+ CD45+ hematopoietic progenitor cells; and
e. culturing the CD34+ CD45+ hematopoietic progenitor cell in a fourth chemically defined culture medium for a sufficient time to produce a CD3− CD56+ natural killer cell.

38. The method of claim 37, wherein the pluripotent stem cell is an embryonic stem cell or an induced pluripotent stem cell.

39. A method for producing the genetically engineered CD3− CD56+ natural killer cell of claim 26, the method comprising:
a. genetically engineering a pluripotent stem cell to express an anti-GD2 CAR; and
b. culturing the pluripotent stem cell in a feeder-free and serum-free medium for a sufficient time to produce the CD3− CD56+ natural killer cell.

40. An isolated population of genetically engineered CD3− CD56+ natural killer cells obtained according to the methods of claim 37.

41. An isolated population of genetically engineered CD3− CD56+ natural killer cells obtained according to the methods of claim 39.

42. A pharmaceutical composition comprising the genetically engineered CD3− CD56+ natural killer cell of claim 26.

43. A method of treating a solid tumor in a subject in need thereof, the method comprising administering a therapeutically effective amount of the pharmaceutical composition of claim 42, and wherein the solid tumor expresses GD2.

44. A method of reducing proliferation or survival of a solid tumor cell, the method comprising contacting the solid tumor with the genetically engineered CD3− CD56+ natural killer cell of claim 26, and wherein the solid tumor expresses GD2.

45. The method of claim 43, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

46. The method of claim 44, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

47. A genetically engineered EPX+ eosinophil, wherein the EPX+ eosinophil expresses an anti-GD2 CAR and is capable of inhibiting tumor cell proliferation or survival of cells expressing GD2 antigen, and wherein the tumor cell expresses GD2.

48. The genetically engineered EPX+ eosinophil of claim 47, wherein the eosinophil is produced from a pluripotent stem cell genetically engineered to express an anti-GD2 CAR.

49. The genetically engineered EPX+ eosinophil of claim 48, wherein the pluripotent stem cell is an embryonic stem cell or an induced pluripotent stem cell.

50. The genetically engineered EPX+ eosinophil of claim 47, wherein the eosinophil selectively targets cells that express GD2 antigen.

51. The genetically engineered EPX+ eosinophil of claim 47, wherein the tumor cell is a solid tumor.

52. The genetically engineered EPX+ eosinophil of claim 47, wherein the tumor cell is not a blood cancer.

53. The genetically engineered EPX+ eosinophil of claim 47, wherein the tumor cell is a neuroblastoma, retinoblastoma, medulloblastoma, glioblastoma, melanoma, lung cancer, pancreatic cancer, bladder cancer, colorectal cancer, sarcoma, or breast cancer cell.

54. The genetically engineered EPX+ eosinophil of claim 47, wherein the anti-GD2 CAR has an amino acid sequence comprising SEQ ID NO:6, or a sequence having at least 80% sequence identity to SEQ ID NO:6.

55. The genetically engineered EPX+ eosinophil of claim 47, wherein the anti-GD2 CAR comprises a nucleic acid sequence encoding an anti-GD2-scFv1 polypeptide, a hinge polypeptide, a CD28 transmembrane polypeptide, an OX40 polypeptide, and a CD3-zeta polypeptide.

56. An isolated population of the genetically engineered EPX+ eosinophils of claim 47.

57. The isolated population of claim 56, wherein the population comprises about 30% to about 40% EPX+ eosinophils.

58. The isolated population of EPX+ eosinophils of claim 57, wherein the population have been further purified to about 90% to about 99% EPX+ eosinophils.

59. A method for producing the genetically engineered EPX+ eosinophil of claim 47, the method comprising:
a. genetically engineering a pluripotent stem cell to express an anti-GD2 CAR;
b. culturing the pluripotent stem cell in a first chemically defined medium for a sufficient time to produce a mesoderm cell;
c. culturing the mesoderm cell seeded at low density in a second chemically defined culture medium that comprises a fibroblast growth factor (FGF) and a vascular endothelial growth factor (VEGF) for a sufficient time to produce a hemogenic endothelial cell;
d. culturing the hemogenic endothelium cell in a third chemically defined culture medium for a sufficient time to produce a CD34+ CD45+ hematopoietic progenitor cells; and
e. culturing the CD34+ CD45+ hematopoietic progenitor cell in a fourth chemically defined culture medium for a sufficient time to produce an EPX+ eosinophil.

60. The method of claim 59, wherein the pluripotent stem cell is an embryonic stem cell or an induced pluripotent stem cell.

61. A method for producing the genetically engineered EPX+ eosinophil of claim 47, the method comprising:
a. genetically engineering a pluripotent stem cell to express an anti-GD2 CAR; and
b. culturing the pluripotent stem cell in a feeder-free and serum-free medium for a sufficient time to produce the EPX+ eosinophil.

62. An isolated population of genetically engineered EPX+ eosinophils obtained according to the method of claim 59.

63. An isolated population of genetically engineered EPX+ eosinophils obtained according to the method of claim 61.

64. A pharmaceutical composition comprising the genetically engineered EPX+ eosinophil of claim 47.

65. A method of treating a solid tumor in a subject in need thereof, the method comprising administering a therapeutically effective amount of the pharmaceutical composition of claim 64, and wherein the solid tumor expresses GD2.

66. A method of reducing proliferation or survival of a solid tumor cell, the method comprising contacting the solid tumor with the genetically engineered EPX+ eosinophil of claim 47, and wherein the solid tumor expresses GD2.

67. The method of claim 65, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

68. The method of claim 66, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

69. A genetically engineered CD11b+ CD15+ neutrophil, wherein the CD11b+ CD15+ neutrophil expresses an anti-disialoganglioside GD2 (GD2) chimeric antigen receptor (CAR) and is capable of inhibiting tumor cell proliferation or survival of cells expressing GD2 antigen, and wherein the tumor cell expresses GD2.

70. The genetically engineered CD11b+ CD15+ neutrophil of claim 69, wherein the neutrophil is produced from a pluripotent stem cell genetically engineered to express an anti-GD2 CAR.

71. The genetically engineered CD11b+ CD15+ neutrophil of claim 70, wherein the pluripotent stem cell is an embryonic stem cell or an induced pluripotent stem cell.

72. The genetically engineered CD11b+ CD15+ neutrophil of claim 69, wherein the neutrophil selectively targets cells that express GD2 antigen.

73. The genetically engineered CD11b+ CD15+ neutrophil of claim 69, wherein the tumor cell is a solid tumor.

74. The genetically engineered CD11b+ CD15+ neutrophil of claim 69, wherein the tumor cell is not a blood cancer.

75. The genetically engineered CD11b+ CD15+ neutrophil of claim 69, wherein the tumor cell is a neuroblastoma, retinoblastoma, medulloblastoma, glioblastoma, melanoma, lung cancer, pancreatic cancer, bladder cancer, colorectal cancer, sarcoma, or breast cancer cell.

76. The genetically engineered CD11b+ CD15+ neutrophil of claim 69, wherein the anti-GD2 CAR has an amino acid sequence comprising SEQ ID NO:6, or a sequence having at least 80% sequence identity to SEQ ID NO:6.

77. The genetically engineered CD11b+ CD15+ neutrophil of claim 69, wherein the anti-GD2 CAR comprises a nucleic acid sequence encoding an anti-GD2-scFv1 polypeptide, a hinge polypeptide, a CD28 transmembrane polypeptide, an OX40 polypeptide, and a CD3-zeta polypeptide.

78. An isolated population of the genetically engineered CD11b+ CD15+ neutrophils of claim 69.

79. The isolated population of claim 78, wherein the population comprises about 90% to about 99% CD11b+ CD15+ neutrophils.

80. A method for producing the genetically engineered CD11b+ CD15+ neutrophil of claim 69, the method comprising:
a. genetically engineering a pluripotent stem cell (PSC) to express an anti-GD2 chimeric antigen receptor (CAR);
b. introducing exogenous ETV2 in the genetically engineered PSC and culturing the ETV2-induced PSC in a xenogen-free, feeder-free, and serum-free medium to produce a population of ETV2-induced endothelial progenitor cells;
c. culturing the ETV2-induced endothelial progenitor cells in xenogen-free, feeder-free, and serum-free medium comprising for a sufficient time to produce non-adherent myeloid progenitors; and
d. culturing the myeloid progenitors in xenogen-free, feeder-free, and serum-free medium for a sufficient time to differentiate the non-adherent myeloid progenitors into CD11b+ CD15+ neutrophils.

81. The method of claim 80, wherein the myeloid progenitors express CD34 and CD45 by day 9 in culture.

82. A pharmaceutical composition comprising the genetically engineered CD11b+ CD15+ neutrophil of claim 69.

83. A method of treating a solid tumor in a subject in need thereof, the method comprising administering a therapeutically effective amount of the pharmaceutical composition of claim 82, and wherein the solid tumor expresses GD2.

84. A method of reducing proliferation or survival of a solid tumor cell, the method comprising contacting the solid tumor with the genetically engineered CD11b+ CD15+ neutrophil of claim 69, and wherein the solid tumor expresses GD2.

85. The method of claim 83, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

86. The method of claim 84, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

87. The method of claim 83, wherein the genetically engineered CD11b+ CD15+ neutrophil secretes inflammatory cytokines.

88. The method of claim 84, wherein the genetically engineered CD11b+ CD15+ neutrophil secretes inflammatory cytokines.

89. A pharmaceutical composition comprising the genetically engineered CD11b+ CD14+ macrophage of claim 1 and a genetically engineered CD3− CD56+ natural killer cell, wherein the natural killer cell expresses an anti-GD2 CAR.

90. A method of treating a solid tumor in a subject in need thereof, the method comprising administering a therapeutically effective amount of the pharmaceutical composition of claim 89, and wherein the solid tumor expresses GD2.

91. A method of reducing proliferation or survival of a solid tumor cell, the method comprising contacting the solid tumor with the genetically engineered CD11b+ CD14+ macrophage of claim 1 and a genetically engineered CD3− CD56+ natural killer cell, wherein the natural killer cell expresses an anti-GD2 CAR, and wherein the solid tumor expresses GD2.

92. The method of claim 90, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

93. The method of claim 91, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

94. The method of claim 90, wherein the genetically engineered CD11b+ CD14+ macrophage promotes the anti-tumor activity of the genetically engineered CD3− CD56+ natural killer cell.

95. The method of claim 91, wherein the genetically engineered CD11b+ CD14+ macrophage promotes the anti-tumor activity of the genetically engineered CD3− CD56+ natural killer cell.

96. The method of claim 90, wherein the genetically engineered CD11b+ CD14+ macrophage increases proliferation of the genetically engineered CD3− CD56+ natural killer cell.

97. The method of claim 91, wherein the genetically engineered CD11b+ CD14+ macrophage increases proliferation of the genetically engineered CD3− CD56+ natural killer cell.

98. A pharmaceutical composition comprising the genetically engineered CD11b+ CD14+ macrophage of claim 1 and a genetically engineered EPX+ eosinophil, wherein the eosinophil expresses an anti-GD2 CAR.

99. A method of treating a solid tumor in a subject in need thereof, the method comprising administering a therapeutically effective amount of the pharmaceutical composition of claim 98, and wherein the solid tumor expresses GD2.

100. A method of reducing proliferation or survival of a solid tumor cell, the method comprising contacting the solid tumor with the genetically engineered CD11b+ CD14+ macrophage of claim 1 and a genetically engineered EPX+ eosinophil, wherein the eosinophil expresses an anti-GD2 CAR, and wherein the solid tumor expresses GD2.

101. The method of claim 99, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

102. The method of claim 100, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

103. A pharmaceutical composition comprising the genetically engineered CD11b+ CD14+ macrophage of claim 1 and a genetically engineered CD11b+ CD15+ neutrophil, wherein the neutrophil expresses an anti-GD2 CAR.

104. A method of treating a solid tumor in a subject in need thereof, the method comprising administering a therapeutically effective amount of the pharmaceutical composition of claim 103, and wherein the solid tumor expresses GD2.

105. A method of reducing proliferation or survival of a solid tumor cell, the method comprising contacting the solid tumor with the genetically engineered CD11b+ CD14+ macrophage of claim 1 and a genetically engineered CD11b+ CD15+ neutrophil, wherein the neutrophil expresses an anti-GD2 CAR, and wherein the solid tumor expresses GD2.

106. The method of claim 104, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

107. The method of claim 105, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

108. A pharmaceutical composition comprising the genetically engineered CD3− CD56+ natural killer cell of claim 26 and a genetically engineered EPX+ eosinophil, wherein the eosinophil expresses anti-GD2 CAR.

109. A method of treating a solid tumor in a subject in need thereof, the method comprising administering a therapeutically effective amount of the pharmaceutical composition of claim 108, and wherein the solid tumor expresses GD2.

110. A method of reducing proliferation or survival of a solid tumor cell, the method comprising contacting the solid tumor with the genetically engineered CD3− CD56+ natural killer cell of claim 26 and a genetically engineered EPX+ eosinophil, wherein the eosinophil expresses anti-GD2 CAR, and wherein the solid tumor expresses GD2.

111. The method of claim 109, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

112. The method of claim 110, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

113. The method of claim 109, wherein the EPX+ eosinophil promotes the anti-tumor activity of the genetically engineered CD3− CD56+ natural killer cell.

114. The method of claim 110, wherein the EPX+ eosinophil promotes the anti-tumor activity of the genetically engineered CD3− CD56+ natural killer cell.

115. A pharmaceutical composition comprising the genetically engineered CD3− CD56+ natural killer cell of claim 26 and a genetically engineered CD11b+ CD15+ neutrophil, wherein the neutrophil expresses an anti-GD2 CAR.

116. A method of treating a solid tumor in a subject in need thereof, the method comprising administering a therapeutically effective amount of the pharmaceutical composition of claim 115, and wherein the solid tumor expresses GD2.

117. A method of reducing proliferation or survival of a solid tumor cell, the method comprising contacting the solid tumor with the genetically engineered CD3− CD56+ natural killer cell of claim 26 and a genetically engineered CD11b+ CD15+ neutrophil, wherein the neutrophil expresses an anti-GD2 CAR, and wherein the solid tumor expresses GD2.

118. The method of claim 116, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

119. The method of claim 117, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

120. A pharmaceutical composition comprising the genetically engineered EPX+ eosinophil of claim 47 and a genetically engineered CD11b+ CD15+ neutrophil, wherein the neutrophil expresses an anti-GD2 CAR.

121. A method of treating a solid tumor in a subject in need thereof, the method comprising administering a therapeutically effective amount of the pharmaceutical composition of claim 120, and wherein the solid tumor expresses GD2.

122. A method of reducing proliferation or survival of a solid tumor cell, the method comprising contacting the solid tumor with the genetically engineered EPX+ eosinophil of claim 47 and a genetically engineered CD11b+ CD15+ neutrophil, wherein the neutrophil expresses an anti-GD2 CAR, and wherein the solid tumor expresses GD2.

123. The method of claim 121, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

124. The method of claim 122, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

125. A pharmaceutical composition comprising the genetically engineered CD11b+ CD14+ macrophage of claim 1, a genetically engineered CD3− CD56+ natural killer cell, a genetically engineered EPX+ eosinophil, and a genetically engineered CD11b+ CD15+ neutrophil, or any combination thereof, wherein the natural killer cell, the eosinophil, and the neutrophil express an anti-GD2 CAR.

126. A method of treating a solid tumor in a subject in need thereof, the method comprising administering a therapeutically effective amount of the pharmaceutical composition of claim 125, and wherein the solid tumor expresses GD2.

127. A method of reducing proliferation or survival of a solid tumor cell, the method comprising contacting the solid tumor with the genetically engineered CD11b+ CD14+ macrophage of claim 1, a genetically engineered CD3− CD56+ natural killer cell, a genetically engineered EPX+ eosinophil, and a genetically engineered CD11b+ CD15+ neutrophil, or any combination thereof, wherein the natural killer cell, the eosinophil, and the neutrophil express an anti-GD2 CAR, and wherein the solid tumor expresses GD2.

128. The method of claim 126, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

129. The method of claim 127, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

130. The method of claim 126, wherein the genetically engineered EPX+ eosinophil, or the CD11b+ CD14+ macrophage promotes the anti-tumor activity of the genetically engineered CD3− CD56+ natural killer cell.

131. The method of claim 127, wherein the genetically engineered EPX+ eosinophil, or the CD11b+ CD14+ macrophage promotes the anti-tumor activity of the genetically engineered CD3− CD56+ natural killer cell.

132. The method of claim 126, wherein the genetically engineered CD11b+ CD14+ macrophage increases proliferation of the genetically engineered CD3− CD56+ natural killer cell.

133. The method of claim 127, wherein the genetically engineered CD11b+ CD14+ macrophage increases proliferation of the genetically engineered CD3− CD56+ natural killer cell.

134. The method of claim 126, wherein the genetically engineered CD11b+ CD15+ neutrophil secretes inflammatory cytokines after co-culture with tumor cells expressing GD2.

135. The method of claim 127, wherein the genetically engineered CD11b+ CD15+ neutrophil secretes inflammatory cytokines after co-culture with tumor cells expressing GD2.

136. The genetically engineered CD11b+ CD14+ macrophage of claim 1, wherein the macrophage has inhibited expression of signal regulatory protein alpha (SIRPa).

137. The genetically engineered CD11b+ CD14+ macrophage of claim 136, wherein the expression of SIRPa is inhibited by gene mutation, RNA-mediated inhibition, RNA editing, DNA gene editing or base editing.

138. The genetically engineered CD11b+ CD14+ macrophage of claim 137, wherein the expression of SIRPa is knocked out by gene editing method.

139. The genetically engineered CD11b+ CD14+ macrophage of claim 138 wherein exon 3 of SIRPa gene is knocked out by gene editing method.

140. The genetically engineered CD11b+ CD14+ macrophage of claim 139, the gene editing method comprises using a nuclease selected from a meganuclease, ZGNs, TALENs, and Cas enzyme.

141. A pharmaceutical composition comprising the genetically engineered CD11b+ CD14+ macrophage of claim 136 and a genetically engineered CD3− CD56+ natural killer cell, wherein the natural killer cell expresses an anti-GD2 CAR.

142. A method of treating a solid tumor in a subject in need thereof, the method comprising administering a therapeutically effective amount of the pharmaceutical composition of claim 141, and wherein the solid tumor expresses GD2.

143. A method of reducing proliferation or survival of a solid tumor cell, the method comprising contacting the solid tumor with the genetically engineered CD11b+ CD14+ macrophage of claim 136 and a genetically engineered CD3− CD56+ natural killer cell, wherein the natural killer cell expresses an anti-GD2 CAR, and wherein the solid tumor expresses GD2.

144. The method of claim 142, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

145. The method of claim 143, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

146. The method of claim 142, wherein the genetically engineered CD11b+ CD14+ macrophage promotes the anti-tumor activity of the genetically engineered CD3− CD56+ natural killer cell.

147. The method of claim 143, wherein the genetically engineered CD11b+ CD14+ macrophage promotes the anti-tumor activity of the genetically engineered CD3− CD56+ natural killer cell.

148. The method of claim 142, wherein the genetically engineered CD11b+ CD14+ macrophage increases proliferation of the genetically engineered CD3− CD56+ natural killer cell.

149. The method of claim 143, wherein the genetically engineered CD11b+ CD14+ macrophage increases proliferation of the genetically engineered CD3− CD56+ natural killer cell.

150. A pharmaceutical composition comprising the genetically engineered CD11b+ CD14+ macrophage of claim 136 and a genetically engineered EPX+ eosinophil, wherein the eosinophil expresses an anti-GD2 CAR.

151. A method of treating a solid tumor in a subject in need thereof, the method comprising administering a therapeutically effective amount of the pharmaceutical composition of claim 150, and wherein the solid tumor expresses GD2.

152. A method of reducing proliferation or survival of a solid tumor cell, the method comprising contacting the solid tumor with the genetically engineered CD11b+ CD14+ macrophage of claim 136 and a genetically engineered EPX+ eosinophil, wherein the eosinophil expresses an anti-GD2 CAR, and wherein the solid tumor expresses GD2.

153. The method of claim 151, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

154. The method of claim 152, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

155. A pharmaceutical composition comprising the genetically engineered CD11b+ CD14+ macrophage of claim 136 and a genetically engineered CD11b+ CD15+ neutrophil, wherein the neutrophil expresses an anti-GD2 CAR.

156. A method of treating a solid tumor in a subject in need thereof, the method comprising administering a therapeutically effective amount of the pharmaceutical composition of claim 155, and wherein the solid tumor expresses GD2.

157. A method of reducing proliferation or survival of a solid tumor cell, the method comprising contacting the solid tumor with the genetically engineered CD11b+ CD14+ macrophage of claim 136 and a genetically engineered CD11b+ CD15+ neutrophil, wherein the neutrophil expresses an anti-GD2 CAR, and wherein the solid tumor expresses GD2.

158. The method of claim 156, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.

159. The method of claim 157, wherein the solid tumor is a neuroblastoma, retinoblastoma, medulloblastoma, glioblastoma, melanoma, lung cancer, pancreatic cancer, bladder cancer, colorectal cancer, sarcoma, or breast cancer cell.


SEQUENCE LISTING (11 sequences)

SEQ ID NO: 1 - DNA, 1494 bp, Synthetic construct
  source 1..1494: mol_type = other DNA
atggccctgcctgtgacagccctgctgctgcctctggctctgctgctgcatgccgctagaccccaggtgaaactgcagcagtcaggacctgaactggtgaagcctggggcttcagtgaagatatcctgcaagacttctggatacaaattcactgaatacaccatgcactgggtgaagcagagccatggaaagagccttgagtggattggaggtattaatcctaacaatggtggtactaactacaagcagaagttcaagggcaaggccacattgactgtagacaagtcctccagcacagcctacatggagctccgcagcctgacatctgaggattctgcagtctattactgtgcaagagatactacggtcccgtttgcttactgggtccaagggaccacggtcaccgtctcctcaggtggaggcggttcaggcggaggtggctctggcggtggcggatcggacatcgagctcactcagtctccagcaatcatgtctgcatctccaggggagaaggtcaccatgacctgcagtggcagctcaagtataagttacatgcactggtaccagcagaagcctgtcacctcccccaaaagatggatttatgacacatccaaactggcttctggagtccctgctcgcttcagtggcagtgggtctgggacctcttattctctcacaatcagcagcatggaggctgtagatgctgccacttattactgccatcagcggagtagttacccgctcacgttcggtgctgggacacagttggaaataaaacgggaacaaaaacttatttctgaagaagatctgaccacgacgccagcgccgcgaccaccaacaccggcgcccaccatcgcgtcgcagcccctgtccctgcgcccagaggcgagtcggccagcggcggggggcgcagtgcacacgagggggctggacttcgcctgtgatatctacatctgggcgcccttggccgggacttgtggggtccttctcctgtcactggttatcaccctttactgcggctcaaaacggggcagaaagaaactcctgtatatattcaaacaaccatttatgagaccagtacaaactactcaagaggaagatggctgtagctgccgatttccagaagaagaagaaggaggatgtgaactgggctcaagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 2 - DNA, 1560 bp, Synthetic construct
  source 1..1560: mol_type = other DNA
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagccaaaacgacacccccatcagtctatggaagggtcaccgtctcttcagcggagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 3 - DNA, 1671 bp, Synthetic construct
  source 1..1671: mol_type = other DNA
atggagttcggcctgtcttggctgtttctggtggccatcctgaagggcgtgcagtgcagcagagacatcctgctgacacagaccccactgagcctgcccgtgagcctgggcgatcaggcctctatcagctgtcgcagctcccagtctctggtgcaccggaacggcaatacctacctgcactggtatctgcagaagccaggccagagccccaagctgctgatccacaaggtgtctaacagattcagcggcgtgcctgacaggttttccggctctggcagcggcacagatttcaccctgaagatctccagagtggaggccgaggacctgggcgtgtacttctgctcccagtctacacacgtgccccctctgacatttggcgccggcaccaagctggagctgaagagggcagatgcagcacctaccgtgagcatctttccaggatccggaggaggaggatctggaggagaggtgaagctgcagcagtctggaccaagcctggtggagcctggagcatccgtgatgatctcttgtaaggccagcggctctagcttcacaggctacaacatgaattgggtgcgccagaacatcggcaagagcctggagtggatcggcgccatcgacccctactatggcggcacctcctacaatcagaagtttaagggccgggccacactgaccgtggacaagtcctctagcacagcctatatgcacctgaagtccctgacctctgaggatagcgccgtgtactattgcgtgagcggcatggagtactggggccagggcacatccgtgaccgtgtcctctgccaagaccacaccacccagcgtgtatggccgcgtgacagtgagctccgccttcgtgcccgtgttcctgcctgccaagccaaccacaacccctgcaccaaggcctccaacaccagcacctaccatcgcatcccagccactgtctctgaggccagaggcatgtaggcctgcagcaggcggcgccgtgcacaccaggggcctggacttcgcctgcgatatctacatctgggcaccactggcaggaacatgtggcgtgctgctgctgtccctggtcatcaccagatccaagcggagccggctgctgcactctgactatatgaacatgacaccacggagaccaggaccaaccaggaagcactaccagccttatgcaccacctagggattttgcagcataccggtccaagagaggcaggaagaagctgctgtatatcttcaagcagccttttatgagaccagtgcagacaacccaggaggaggacggctgcagctgtaggttcccagaagaggaggagggaggatgcgagctgagagtgaagtttagcaggtccgccgatgcaccagcataccagcagggacagaatcagctgtataacgagctgaatctgggcaggcgcgaggagtacgacgtgctggataagaggagaggaagggatccagagatgggaggcaagccaaggcgcaagaacccccaggagggcctgtacaatgagctgcagaaggacaagatggccgaggcctattccgagatcggcatgaagggagagcggagaaggggcaagggacacgatggcctgtaccagggcctgagcacagccaccaaggacacctatgatgcactgcacatgcaggccctgccaccaaggtga

SEQ ID NO: 4 - DNA, 22 bp, Synthetic construct
  source 1..22: mol_type = other DNA
atcggtgcgggcctcttcgcta

SEQ ID NO: 5 - protein, 497 aa, Synthetic construct
MALPVTALLLPLALLLHAARPQVKLQQSGPELVKPGASVKISCKTSGYKFTEYTMHWVKQSHGKSLEWIGGINPNNGGTNYKQKFKGKATLTVDKSSSTAYMELRSLTSEDSAVYYCARDTTVPFAYWVQGTTVTVSSGGGGSGGGGSGGGGSDIELTQSPAIMSASPGEKVTMTCSGSSSISYMHWYQQKPVTSPKRWIYDTSKLASGVPARFSGSGSGTSYSLTISSMEAVDAATYYCHQRSSYPLTFGAGTQLEIKREQKLISEEDLTTTPAPRPPTPAPTIASQPLSLRPEASRPAAGGAVHTRGLDFACDIYIWAPLAGTCGVLLLSLVITLYCGSKRGRKKLLYIFKQPFMRPVQTTQEEDGCSCRFPEEEEGGCELGSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 696 aa, Synthetic construct
MEFGLSWLFLVAILKGVQCSRDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSAEPKSCDKTHTCPPCPDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRQPDAIHSSGAGCLSEGGGWCGQCPGSQIPLRSFSLCQKLWGHHEAPASDFWLIKEIYFHCNSVLEFFVSLTRKDIWEGKSFKTSEVFGLEFGNICPYAGCHEQRLAIKRSSVYETAPCCPFLIPKSLDLRLDFFYILFCVIFFFNIPKIFLTCFTSQIFPPLLTTPSHSCPSSLMEI

SEQ ID NO: 7 - protein, 556 aa, Synthetic construct
MEFGLSWLFLVAILKGVQCSRDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSAFVPVFLPAKPTTTPAPRPPTPAPTIASQPLSLRPEACRPAAGGAVHTRGLDFACDIYIWAPLAGTCGVLLLSLVITRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - RNA, 20 nt, Synthetic construct
  source 1..20: mol_type = other RNA
gtgctcctttccaggagtgg

SEQ ID NO: 9 - RNA, 20 nt, Synthetic construct
  source 1..20: mol_type = other RNA
acttaaactccacgtcatcg

SEQ ID NO: 10 - RNA, 20 nt, Synthetic construct
  source 1..20: mol_type = other RNA
ccctcctcgctccgcagccg

SEQ ID NO: 11 - RNA, 20 nt, Synthetic construct
  source 1..20: mol_type = other RNA
ccgctgcactccccaaactg